(19) United States
(12) Patent Application Publication
(10) Pub. No.: US 2015/0202280 A1
(43) Pub. Date: Jul. 23, 2015

(54) Mutant Forms of Chlamydia HtrA

(71) Applicant: Novartis AG, Basel (CH)

(72) Inventors: Roberto PETRACCA, Siena (IT); Renata Maria GRIFANTINI, Siena (IT); Guido GRANDI, Segrate (IT)

(21) Appl. No.: 14/627,270

(22) Filed: Feb. 20, 2015

Related U.S. Application Data
(62) Division of application No. 12/921,271, filed on Nov. 23, 2010.
(60) Provisional application No. 61/034,212, filed on Mar. 6, 2008.

(51) Int. Cl. A61K 39/118; C07K 16/12

(52) U.S. Cl. CPC A61K 39/118; C07K 16/125; C07K 2317/76

(57) ABSTRACT

An immunogenic Chlamydia HtrA protein, which has one or more mutations relative to wild-type Chlamydia HtrA that result in a reduced or eliminated protease activity relative to the protease activity of wild-type Chlamydia HtrA. Preferably, it is the serine protease activity that is reduced or eliminated.

This application incorporates by reference the contents of a 170 kb text file created on Feb. 20, 2015 and named “PAT052595sequencelisting.txt,” which is the sequence listing for this application.

### TECHNICAL FIELD

This invention is in the field of Chlamydia HtrA proteins and their uses.

### BACKGROUND ART

Vaccine development has been identified as essential to controlling infection with C. trachomatis. Vaccines against C. trachomatis appear to elicit protective T-cell and/or B-cell immunity in the genital tract mucosa. In particular, protection in an infection-animal model seems to be mediated by CD4+ T cells that produce IFN-γ. Although B-cells and antibodies do not have a decisive role in resolution of primary infection, they might be important for enhancing the protective effector T-cell response and be required to control re-infection with various mechanisms such as antibody-mediated neutralization and opsonization.

Because immune protection against infection with C. trachomatis is likely to be mediated by immunization with C. trachomatis proteins that are targets of CD4+ T cells and that are capable of inducing B-cell responses, identification of such proteins is particularly important. Numerous studies on the most promising vaccine candidate (Major Outer Membrane Protein, MOMP) have shown that an effective vaccine is likely to be based on several C. trachomatis antigens. It is therefore an object of the invention to provide further antigens for use in Chlamydia vaccines.

The homologue proteins CT823 of Chlamydia trachomatis (Ct) and TC0210 of Chlamydia muridarum (Cm) are annotated as serine proteases and share a 93.36 percent sequence identity. Together with the high temperature requirement A (HtrA) protein of E. coli and the homologues in other bacteria and eukaryotes, these proteins constitute the HtrA protease family. The chief role of these proteases is to degrade misfolded proteins in the periplasm (Lipinska, B. et al., J. Bacteriol., 172, 1791-1797; Gray, C. W. et al., Eur. J. Biochem., 2000, 267, 5699-5710; Savopoulos, J. W. et al., Protein Expres. Purif., 2000, 19, 227-234). HtrA from Chlamydia trachomatis (also referred to herein as “CtHtrA”) has been characterised as a serine endoprotease, specific for unfolded proteins, which is temperature activated above 34° C. (Huston, W. M. et al., FEES Letters, 2007, 3382-3386). Chaperone activity has been observed, although this appears to be target-dependent.

Previous studies, with mass spectrometric and cytofluorimetric analysis on CtHtrA have confirmed its localization on the surface of the bacterium (WO03/049762). The CtHtrA antigen is able to induce a specific CD4-Th1 response in splenocytes isolated from mice infected with C. trachomatis and has been predicted to contain MHC class II epitopes (see WO2006/138004 and WO2007/110700). it has also been found to have neutralising activity (see WO2007/110700). Thus, the Chlamydia HtrA protein is a promising antigen candidate for development of a vaccine.

### DISCLOSURE OF THE INVENTION

In vitro proteolytic assays with wild type C. trachomatis HtrA serine protease (“CT823”) incubated with different substrates show degradation of proteins, such as bovine serum albumin (“BSA”), actin and other important antigens that may be used to develop vaccines, such as MOMP. Thus, if wild-type HtrA were present in a vaccine formulation against Chlamydia infection, it may damage host proteins or other components of the vaccine. This would cause serious problems for the safety and efficacy of the vaccine. It is therefore an object of the invention to provide an HtrA antigen for use in an improved vaccine formulation that does not cause proteolysis of host proteins or of other antigens in the vaccine composition.

The invention therefore provides an immunogenic Chlamydia HtrA protein, which has one or more mutations relative to wild-type Chlamydia HtrA that result in a reduced or eliminated protease activity relative to the protease activity of wild-type Chlamydia HtrA. Preferably, it is the serine protease activity that is reduced or eliminated.

The term “immunogenic” in the context of “an immunogenic HtrA protein”, is used to mean that the protein is capable of eliciting an immune response, such as a cell-mediated and/or an antibody response, against the wild-type Chlamydia HtrA protein from which it is derived, for example, when used to immunise a subject (preferably a mammal, more preferably a human or a mouse). For example, the protein of the invention is preferably capable of stimulating in vitro CD4+ IFN+−γ cells in splenocytes purified from mice infected with live C. trachomatis to a level comparable with the wild-type Chlamydia HtrA, The protein of the invention preferably retains the ability to elicit antibodies that recognise the wild-type HtrA. For example, the protein of the invention preferably elicits antibodies that can bind to, and preferably neutralise the proteolytic activity of, the wild-type HtrA protein. In a further embodiment, the protein of the invention is capable of eliciting antibodies that are capable of neutralising Chlamydia infectivity and/or virulence. In some embodiments, the antibodies are able to cross-react with the protein of the invention and the wild-type HtrA, but with no other HtrA (e.g. HtrA from E. coli or H. influenzae or from another Chlamydia species). In other embodiments, the antibodies are cross-reactive with the wild-type HtrA and with HtrA from other Chlamydia species. In sonic embodiments, the antibodies are cross reactive with the wild-type HtrA and with HtrA from other organisms (for example from E. coli or H. influenzae). Mice immunized with the protein of the invention and the wild-type Chlamydia HtrA preferably show similar antigen-specific antibody titers. Antibody titres and specificities can be measured using standard methods available in the art. Other methods of testing the immunogenicity of proteins are also well known in the art.

The wild-type HtrA is preferably from C. trachomatis. The human serovariants (“serovars”) of C. trachomatis are divided into two biovariants (“biovars”). Serovars A-K elicit epithelial infections primarily in the ocular tissue (A-C) or urogenital tract (D-K). Serovars L1, L2 and L3 are the agents of invasive lymphogranuloma venereum (LGV). The wild type HtrA may, for example, be of any of Serovars A-K or L1, L2 or L3. Preferably, the wild-type HtrA is from C. trachomatis serovar D, or from another epidemiologically prevalent serotype. Most preferably, the amino acid and/or nucleic acid sequence of the wild-type HtrA protein from C. trachomatis comprises or consists of the sequence presented in SEQ ID NO:1 and SEQ ID NO:2 respectively. This protein is also known as “CT823”. Alternatively, the wild-type HtrA may, for example, be from C. pneumoniae, C. psittaci, C. pecorum, C. muridarum (“TC0210”, SEQ ID NO:3) or C. suis.

The reduction or elimination of protease activity is conferred by at least one (for example, 1, 2, 3, 4, 5, 6, 7, 8, 9, 10 or more or all) of the one or more mutations. Preferred mutations are those which reduce or eliminate the protease activity without causing a significant conformational change in the protein such that the protein of the invention retains the ability to elicit an immune response against the wild-type Chlamydia HtrA protein.

Preferred mutations are in the protease domain of Chlamydia HtrA which spans about 180 amino acids. Based on similarity searches between SEQ ID NO:1 and the sequences that are available in the databases, the inventors have found that the protease domain resides in the N-terminal portion of HtrA and that it contains a characteristic triad. According to GenBank (ID AAC68420.1), the protease domain of CT823 belongs to the Trypsin Pfam (Pfam is a database of protein domain families) and spans from amino acid residues 128 to 265. The Kyoto Encyclopedia of Genes and Genomes (KEGG; www.genome.jp/kegg/) reports that the protease domain of CT823 belongs to the Trypsin Pfam and spans from amino acid residues 110 to 288. KEGG also reports that the protease domain of CT823 belongs to the Pfam of the Strep_his_triad and spans from amino acid residues 133 to 183. The alignment of FIG. 9 suggests that the protease domain spans residues 113 to 288 of SEQ ID NO:1 (CT823). Thus, in some embodiments, the one or more mutations that reduce or eliminate the protease activity are present in the N-terminal portion of HtrA, for example, between residues 1 and 288 of SEQ ID NO:1, between residues 110 and 288 of SEQ ID NO:1, between residues 113 and 288 of SEQ ID NO:1; between residues 128 to 265 of SEQ ID NO:1 or between residues 133 and 183 of SEQ ID NO:1, or in a corresponding sequence from the protease domain of another Chlamydia HtrA, as determined using sequence alignment techniques.

More preferably, at least one of the residues in the catalytic triad of the serine protease is mutated (Brenner, S., Nature, 1988, 334, 528-530; Skorko-Glonek, J. Gene, 1995, 163, 47-52). The catalytic triad comprises a His, an Asp and a Ser residue. The protease domain of SEQ ID NO:1 contains a catalytic triad of His143, Asp157 and Ser247 (Huston, W. M. et al., EBBS Letters, 2007, 3382-3386). The skilled person will be able to determine the position of the residues of the catalytic triad in other Chlamydia species, for example, by using sequence alignment techniques. In some embodiments, the His of the catalytic triad is mutated but the Asp and Ser in the catalytic triad are not mutated. In some embodiments, the Asp of the catalytic triad is mutated but the His and the Ser in the catalytic triad are not mutated. In some embodiments, the Ser of the catalytic triad is mutated but the His and the Asp in the catalytic triad are not mutated. In other embodiments, two or more of the residues of the catalytic triad are mutated (e.g. His and Asp, His and Ser, Asp and Ser). In an alternative embodiment, the His, Asp and Ser of the catalytic triad are mutated.

In some embodiments, residues in close proximity to the residues of the catalytic triad in the three dimensional conformation of the HtrA protein may alternatively or additionally be mutated (for example H142). In some embodiments, one or more amino acids that reside close to the amino acids of the catalytic triad in the primary structure are mutated. In sonic embodiments, one or more amino acids that are conserved across species are mutated. For example, it is apparent from the sequence alignment shown in FIG. 9 that residues 127, 129, 131, 138, 140, 141, 143, 144, 145, 148, 151, 153, 166, 167, 169, 173, 175, 191, 198, 199, 203, 204, 205, 207, 209, 214, 215, 217, 218, 219, 220, 236, 237, 238, 239, 242, 243, 245, 246, 247, 248, 249, 251, 253, 256, 259, 260, 261, 262, 274, 277, 278, 279 and 280 of SEQ ID NO:1 are conserved across HtrA from many different species.

Preferably, the amino acid sequences contain fewer than twenty mutations (e.g. 19, 18, 17, 16, 15, 14, 13, 12, 11, 10, 9, 8, 7, 6, 5, 4, 3, 2 or 1). Each mutation preferably involves a single amino acid and is preferably a point mutation. The mutations may each independently be a substitution, an insertion or a deletion. Preferred mutations are single amino acid substitutions. The polypeptides may also include one or more (e.g. 1, 2, 3, 4, 5, 6, 7, 8, 9, etc.) single amino acid deletions relative to the Chlamydia sequences. The polypeptides may also include one or more (e.g. 1, 2, 3, 4, 5, 6, 7, 8, 9, etc) insertions (e.g. each of 1, 2, 3, 4 or 5 amino acids) relative to the Chlamydia sequences. Deletions, substitutions or insertions may be at the N-terminus and/or C-terminus, or may be between the two termini. Thus a truncation is an example of a deletion. Truncations may involve deletion of up to 40 (or more) amino acids at the N-terminus and/or C-terminus.

Amino acid substitutions may be to any one of the other nineteen naturally occurring amino acids. In one embodiment, one or more of the one or more mutations that confers the reduction or elimination of protease activity is a conservative substitution. In another embodiment, one or more of the one or more mutations that confers the reduction or elimination of the protease activity is a non-conservative substitution. A conservative substitution is commonly defined as a substitution introducing an amino acid having sufficiently similar chemical properties, e.g. having a related side chain (e.g. a basic, positively charged amino acid should be replaced by another basic, positively charged amino acid), in order to preserve the structure and the biological function of the molecule. Genetically-encoded amino acids are generally divided into four families: (1) acidic i.e. aspartate, glutamate; (2) basic i.e. lysine, arginine, histidine: (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. Further examples of conversative substitutions that may be used in the invention are presented in Table I.

TABLE 1

| Amino Acid | Synonymous Groups | More Preferred Synonymous Groups |
|---|---|---|
| Ser | Gly, Ala, Ser, Thr, Pro | Thr, Set |
| Arg | Asn, Lys, Gln, Arg, His | Arg, Lys, His |
| Leu | Phe, Ile, Val, Leu, Met | Ile, Val, Leu, Met |
| Pro | Gly, Ala, Ser, Thr, Pro | Pro |
| Thr | Gly, Ala, Ser, Thr, Pro | Thr, Ser |
| Ala | Gly, Thr, Pro, Ala, Ser | Gly, Ala |
| Val | Met, Phe, Ile, Leu, Val | Met, Ile, Val, Leu |
| Gly | Ala, Thr, Pro, Set, Gly | Gly, Ala |
| Ile | Phe, Ile, Val, Leu, Met | Ile, Val, Leu, Met |
| Phe | Trp, Phe, Tyr | Tyr, Phe |
| Tyr | Trp, Phe, Tyr | Phe, Tyr |
| Cys | Ser, Thr, Cys | Cys |
| His | Asn, Lys, Gln, Arg, His | Arg, Lys, His |
| Gln | Glu, Asn, Asp, Gln | Asn, Gln |
| Asn | Glu, Asn, Asp, Gln | Asn, Gln |
| Lys | Asn, Lys, Gln, Arg, His | Arg, Lys, His |
| Asp | Glu, Asn, Asp, Gln | Asp, Glu |
| Glu | Glu, Asn, Asp, Gln | Asp, Glu |
| Met | Phe, Ile, Val, Leu, Met | Ile, Val, Leu, Met |
| Trp | Trp, Phe, Tyr | Trp |

A histidine to arginine subsitution is particularly preferred. For example, both histidine and arginine are polar and basic and their R groups have a similar size. Thus, substituting a histidine with an arginine is a preferred conservative substitution.

Further examples of useful mutations are the substitution of a histidine (H) with a glycine (G), alanine (A), serine (S), valine (V) or threonine (T), more preferably with a lysine (K), glutamine (Q) or asparagine (N); substitution of an aspartatic acid (D) with a threonine, serine, valine, glycine, alanine or glutamic acid (E); substitution of a serine (S) with a glycine (G), valine (V), asparagine (N) or aspartic acid (D), more preferably with an alanine (A) or a threonine (T).

Examples of non-conservative substitutions that may be used in the invention include the substitution of an uncharged polar amino acid with a nonpolar amino acid, the substitution of a nonpolar amino acid with an uncharged polar amino acid, the substitution of an acidic amino acid with a basic amino acid and the substitution of a basic amino acid with an acidic amino acid.

Examples of mutations of residues in the catalytic triad are H-143R, H-143K, D157E. S247A and S247T. H143R and S247A are preferred, with H143R being particularly preferred. Examples of mutations of residues near the catalytic triad are H142R and H142K, with H142R being preferred. Equivalent mutations of the corresponding residues in the HtrA proteins of other Chlamydia species are also envisaged. A particularly preferred protein of the invention is derived from C. trachomatis CT823 and comprises the H143R mutation. More preferably, the protein of the invention comprises or consists of the sequence provided in SEQ ID NO:5. Alternatively, the protein of the invention may be derived from a C. muridarum TC0210 of SEQ ID NO:3 and comprise the H143R mutation.

An HtrA protease from C. trachomatis serovar L2 having a S247A mutation is disclosed in Huston, W. M. et al. (FEBS Letters, 581, 2007, 3382-3386). Therefore, in some embodiments, a C. trachomatis HtrA protein of SEQ ID NO:4 is specifically excluded from the scope of the invention. FIG. 10 shows where differences lie between the sequence of SEQ ID NO:4 (subject sequence, bottom line) and the wild-type CT823 protein of SEQ NO:1 (query sequence, top line). In another embodiment, a C. trachomatis serovar L2 HtrA protein having a S247A mutation is specifically excluded from the scope of the invention. In other embodiments, a C. trachomatis HtrA protein having a S247A mutation is specifically excluded from the scope of the invention. In still further embodiments, a Chlamydia HtrA protein (for example, from C. trachomatis) having a mutation of the serine residue of the catalytic triad is excluded from the scope of the invention.

However, there is no suggestion in Huston, W. M. et al. that the S247A mutant retains its immunogenicity and thus may be used in a vaccine. Indeed, Huston, W. M. et al. explain that at 30° C., only wild-type HtrA and not the S247A displayed significant chaperone activity for α-lactalbumin. The data indicated that chaperone activity may involve a functional protease domain. Thus, by inactivating the protease domain, Huston, W. M. et al. suggests that other functions of the protein are affected. Huston, W. M. et al. provides no suggestion that the immunogenicity will be retained in the S247A inactive protease. Thus although proteins and their encoding nucleic acids) having a mutation of the serine in the catalytic triad may be excluded from the scope of the invention (as described in more detail above), it is envisaged that the uses of the serine mutants in immunogenic compositions of the invention may be encompassed, if desired.

Mutants of HtrA proteins from other organisms that lack protease activity are known. For example, H91A and S197A mutants of Haemophilus influenzae have been examined. However, the S197A mutant has been found to have a more random secondary structure compared to wild-type rHtrA or H91A and to lack immunoprotective properties in a chinchilla model of otitis media (Cates, G. A. et al. Dev. Biol (Basel). 2000; 131:201-4), Thus, mutating a residue of the catalytic triad was found to alter the conformation of the HtrA protein in Haemophilus influenzae and so there was no reason to expect that immunogenicity would be retained when a residue of the catalytic triad is mutated in Chlamydia.

The H91A mutant of H. influenzae HtrA, which lacks the endogenous serine protease activity of wild-type HtrA, has been found to be partially protective in an animal model of invasive H. influenzae type b disease and otitis media (see Loosmore, S. M. et al., Infection and Immunity, 1998, 899-906). However, the level of sequence identity between a H. influenzae HtrA from each of NTH1 Strain 33 (Genbank AF018152) and NTH1 strain 12 (Genbank AF018151) and the C. trachomatis HtrA protein CT823 (SEQ ID NO:1) is only 36%. Further, the mechanism of infection of the H. influenzae is not comparable to the mechanism of infection of Chlamydia. In contrast to H. influenzae, C. trachomatis is an obligate intracellular pathogen that, in its elementary body “EB” infectious form, infects primarily epithelial cells through an endocytosis mechanism. Inside epithelial cells. Chlamydia spp. undergoes a unique biphasic developmental cycle within a specialized vacuole termed an inclusion. H. influenzae does not do this. Moreover, immune mechanisms against Chlamydia and H. influenzae differ significantly. Protection against H. influenzae is mediated mainly by functional antibodies, whereas protection against Chlamydia involves primarily a CD4-Th1 response. Thus the skilled person would expect the immunoprotective properties of homologous antigens from H. influenzae and Chlamydia to differ significantly. Thus, the finding that the H191A mutant of H. influenzae partially retains its immunogenicity is not predictive for the proteins of the present invention and their ability to mediate immunogecitiy and protection in Chlamydia.

Further, in other bacteria, functional HtrA forms multi-subunit complexes. Thus, there would have been a reasonable expectation that mutating the Chlamydia HtrA to reduce or eliminate its protease activity would change the quaternary structure or the stability of the Chlamydia HtrA complexes. Thus, it is particularly surprising that HtrA proteins whose protease activity has been reduced or eliminated by way of mutation in accordance with the present invention retain their immunogenicity.

The Chlamydia HtrA protein of the invention may optionally comprise one or more mutations that do not affect the protease activity in addition to the one or more mutations that confer the reduced or eliminated protease activity. For example, mutations may also be introduced to improve stability, e.g., the insertion of disulphide bonds (van den Akker et al. Protein Sci., 1997, 6:2644-2649). For example, the wild-type Chlamydia HtrA protein may comprise an amino acid sequence having sequence identity to the amino acid sequence recited in SEQ ID NO:1. The degree of sequence identity is preferably greater than 50% (e.g. 60%, 70%, 80%, 90%, 95%, 97%, 98%, 99% or more). These proteins include homologs, orthologs, allelic variants and functional mutants. Identity between proteins is preferably determined by the Smith-Waterman homology search algorithm as implemented in the MPSRCH program (Oxford Molecular), using an affine gap search with parameters gap open penalty=12 and gap extension penalty=1.

The Chlamydia HtrA protein of the invention may comprise one or more amino acid derivatives. By “amino acid derivative” is intended an amino acid or amino acid-like chemical entity other than one of the 20 genetically encoded naturally occurring amino acids. In particular, the amino acid derivative may contain substituted or non-substituted, linear, branched, or cyclic alkyl moieties, and may include one or more heteroatoms. The amino acid derivatives can be made de novo or obtained from commercial sources (Calbiochem-Novabiochem AG, Switzerland; Bachem, USA).

The invention preferably provides a Chlamydia HtrA polypeptide whose proteolytic activity has been reduced by at least 20% (more preferably, at least 30%, at least 40%, at least 50%, at least 60%, at least 70%, at least 80%, at least 90%, at least 95%, at least 98%) relative to the wild-type Chlamydia HtrA. More preferably, the proteolytic activity has been reduced by 100% relative to wild-type Chlamydia HtrA, i.e. has been eliminated.

Protease activity of a protein of the invention and/or of wild-type Chlamydia HtrA may be assayed by performing a digestion consisting of the following steps:

1. mixing a wild type or mutant HtrA protein with a target protein (substrate, such as BSA) in the presence or absence of a reducing agent (such as DTT);

2. incubating the mixture overnight at 37° C.;

3. separating the resulting proteins by means of polyacrylamide gel electrophoresis (SDS-Page);

4. staining the gels with COOMASSIE R-250 BRILLIANT BLUE®; and

5. evaluating the results. For example, digestion of the target protein indicates that the HtrA protease is active, whereas non-digestion of the target protein indicates that the HtrA protease is inactive. Further, the number of moles of target protein that are digested correlates with the activity of the protease.

1. The Chlamydia HtrA proteins having reduced or eliminated protease activity that are defined above are collectively referred to hereafter as the “proteins of the invention”.

The invention further provides a protein comprising or consisting of a fragment of a protein of the invention, wherein the fragment comprises the one or more mutations. The fragment should comprise at least n consecutive amino acids from the protein and, depending on the particular sequence, n is 50 or more (e.g. 60, 90, 120, 150, 180, 210, 240, 270, 300, 330, 360, 390, 420, 450, 480 or more). The fragment is 496 amino acids or less in length (e.g. 485 amino acids or less, 445 amino acids or less, 400 amino acids or less, 350 amino acids or less, 295 amino acids or less, 250 amino acids or less, 200 amino acids or less, or 160 amino acids or less in length). Preferably the fragment comprises one or more epitopes from the protein. The fragment is preferably immunogenic. For example, the fragment is preferably capable of eliciting an immune response, such as a cell-mediated and/or an antibody response, against the wild-type Chlamydia HtrA protein. In one embodiment the fragment is capable of stimulating in vitro CD4+ IFN+−γ cells in splenocytes purified from mice infected with live C. trachomatis to a level comparable with the wild-type Chlamydia HtrA and/or retains the ability to elicit antibodies that recognise the wild-type HtrA.

The proteins of the invention can, of course, be prepared by various means (e.g. recombinant expression, purification from native host, purification from cell culture, chemical synthesis etc.) and in various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.), Generally, the recombinant fusion proteins of the present invention are prepared as a GST-fusion protein and/or a His-tagged fusion protein.

The proteins of the invention are preferably prepared in purified or substantially pure form (i.e. substantially free from host cell proteins and/or other Chlamydia proteins), and are generally at least about 50% pure (by weight), and usually at least about 90% pure, i.e. less than about 50%, and more preferably less than about 10% (e.g. 5%) of a composition is made up of other expressed polypeptides. Thus the antigens in the compositions are separated from the whole organism with which the molecule is expressed.

Whilst expression of the proteins of the invention may take place in Chlamydia, the invention preferably utilises a heterologous host. The heterologous host may be prokaryotic (e.g. a bacterium) or eukaryotic. It is preferably E. coli, but other suitable hosts include Bacillus subtilis, Vibrio cholerae, Salmonella typhi, Salmonella typhimurium, Neisseria lactamica, Neisseria cinerea, Mycobacteria (e.g. M. tuberculosis), yeasts, etc.

The term “polypeptide” or “protein” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included are, for example, polypeptides containing one or more analogs of an amino acid (including, for example, unnatural amino acids, etc.), as well as other modifications known in the art. Polypeptides can occur as single chains or associated chains.

The invention provides polypeptides comprising a sequence —P—Q— or —Q—P—, wherein: —P— is an amino acid sequence as defined above and —Q— is not a sequence as defined above i.e. the invention provides fusion proteins. Where the N-terminus codon of —P— is not ATG, but this codon is not present at the N-terminus of a polypeptide, it will be translated as the standard amino acid for that codon rather than as a Met. Where this codon is at the N-terminus of a polypeptide, however, it will be translated as Met. Examples of —Q— moieties include, but are not limited to, histidine tags (i.e. His where n=3, 4, 5, 6, 7, 8, 9, 10 or more), maltose-binding protein, or glutathione-S-transferase (GST).

Proteins of the invention may be attached to a solid support. They may comprise a detectable label (e.g. a radioactive or fluorescent label, or a biotin label).

### Nucleic Acids

According to a further aspect, the invention provides a nucleic acid encoding a protein of the invention. In some embodiments, the nucleic acid sequence encoding a wild-type Chlamydia HtrA preferably comprises or consists of SEQ ID NO:2.

The invention also provides nucleic acid comprising nucleotide sequences having sequence identity to such nucleotide sequences. Identity between sequences is preferably determined by the Smith-Waterman homology search algorithm as described above. Such nucleic acids include those using alternative codons to encode the same amino acid. These nucleotide sequences having sequence identity retain the ability to encode the one or more mutated residues in the protein of the invention that confer the reduced or eliminated protease activity.

The invention also provides nucleic acid which can hybridize to these nucleic acids. Hybridization reactions can be performed under conditions of different “stringency”. Conditions that increase stringency of a hybridization reaction of widely known and published in the an (e.g. page 7.52 of Kaplitt, Nature Genetics (1994) 6:148). Examples of relevant conditions include (in order of increasing stringency): incubation temperatures of 25° C., 37° C. 50° C., 55° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 initiates; and wash solutions of 6×SSC, 1×SSC, 0.1×SSC, or de-ionized water. Hybridization techniques and their optimization are well known in the art (e.g. see U.S. Pat. No. 5,707,829, Current Protocols in Molecular Biology (F. M. Ausubel et al. eds., 1987) Supplement 30, Kaplitt, Nature Genetics (1994) 6:148, and WO 94/03622, etc.].

The nucleic acid may be used in hybridisation reactions (e.g. Northern or Southern blots, or in nucleic acid microarrays or ‘gene chips’) or in amplification reactions (e.g. PCR, SDA, SSSR, LCR, NASBA, TMA) etc.

The invention also provides a nucleic acid comprising sequences complementary to those described above (e.g. for antisense or probing, or for use as primers). In one embodiment, the nucleic acid is complementary to the full length of the nucleic acid described above.

Nucleic acid according to the invention may be labelled e.g. with a radioactive or fluorescent label. This is particularly useful where the nucleic acid is to be used as a primer or probe e.g. in PCR, LCR or TMA.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus the invention includes mRNA, ribozymes, DNA, cDNA, recombinant nucleic acids, branched nucleic acids, plasmids, vectors, probes, primers, etc., Where nucleic acid of the invention takes the form of RNA, it may or may not have a 5′ cap.

Nucleic acids of the invention can take various forms (e.g. single stranded, double stranded, vectors, primers, probes etc.). Unless otherwise specified or required, any embodiment of the invention that utilizes a nucleic acid may utilize both the double-stranded form and each of two complementary single-stranded forms which make up the double-stranded form. Primers and probes are generally single-stranded, as are antisense nucleic acids.

Nucleic acids of the invention are preferably prepared in substantially pure form (i.e. substantially free from naturally-occurring nucleic acids, particularly from chlamydial or other host cell nucleic acids), generally being at least about 50% pure (by weight), and usually at least about 90% pure.

Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases Of polymerases), from genomic or cDNA libraries, etc.

The invention provides vectors comprising nucleotide sequences of the invention (e.g. cloning or expression vectors) and host cells transformed with such vectors. Nucleic acids of the invention may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector. Preferred vectors are plasmids.

Also provided is a host cell comprising a nucleic acid of the invention. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention, for example, with a vector of the invention.

Where a nucleic acid is DNA, it will be appreciated that “U” in a RNA sequence will be replaced by “T” in the DNA. Similarly, where a nucleic acid is RNA, it will be appreciated that “T” in a DNA sequence will be replaced by “U” in the RNA.

The term “complement” or “complementary” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T).

Nucleic acids of the invention can be used, for example: to produce polypeptides; as hybridization probes for the detection of nucleic acid in biological samples; to generate additional copies of the nucleic acids; to generate ribozymes or antisense oligonucleotides; as single-stranded DNA primers or probes; or as triple-strand forming oligonucleotides.

The invention provides a process for producing nucleic acid of the invention, wherein the nucleic acid is synthesised in part or in whole using chemical means.

A nucleic acid that encodes antibody of the present invention is also provided.

For certain embodiments of the invention, nucleic acids are preferably at least 150 nucleotides in length (e.g. 180, 250, 350, 500, 700, 900, 1100, 1300 nucleotides or longer).

For certain embodiments of the invention, nucleic acids are preferably at most 1491 nucleotides in length (e.g. 1450, 1300, 1150, 1000, 850, 700, 500 nucleotides or shorter).

Primers and probes of the invention, and other nucleic acids used for hybridization, are preferably between 10 and 30 nucleotides in length (e.g. 10, 11, 12, 13, 14, 15, 16, 17, 18, 19, 20, 21, 22, 23, 24, 25, 26, 27, 28, 29, or 30 nucleotides).

### Antibodies

The HtrA wild type and the mutant forms of the invention induce functional antibodies able to neutralize the proteolytic activity of the wild-type HtrA. Neutralizing antibodies may be used as a vaccine capable of neutralising the activity of native HtrA expressed by infectious EB.

According to a further aspect, the invention provides one or more antibodies which binds to a protein of the invention, but which does not bind to the wild type HtrA. In some embodiments, the antibody does not bind to any wild-type Chlamydia HtrA.

The term “antibody” includes intact immunoglobulin molecules, as well as fragments thereof which are capable of binding an antigen. These include hybrid (chimeric) antibody molecules (Winter et al., (1991) Nature 349:293-99; U.S. Pat. No. 4,816,567); F(ab′)2 and F(ab) fragments and Fv molecules; non-covalent heterodimers (Inbar et al., (1972) Proc. Natl. Acad. Sci. U.S.A. 69:2659-62; Ehrlich et al., (1980) Biochem 19:4091-96); single-chain Fv molecules (sFv) (Huston et al., (1988) Proc. Natl. Acad. Sci. U.S.A. 85:5897-83); dimeric and trimeric antibody fragment constructs; minibodies Pack et al., (1992) Biochem 31, 1579-84; Cumber et al., (1992) J. Immunology 149B, 120-26); humanized antibody molecules (Riechmann et al., (1988) Nature 332, 323-27; Verhoeyan et al., (1988) Science 239, 1534-36; and GB 2,276,169); and any functional fragments obtained from such molecules, as well as antibodies obtained through non-conventional processes such as phage display. Preferably, the antibodies are monoclonal antibodies. Methods of obtaining monoclonal antibodies are well known in the art. Humanised or fully-human antibodies are preferred.

The antibodies may be polyclonal or monoclonal and may be produced by any suitable means. The antibody may include a detectable label.

Also provided is a method for preparing antibodies comprising immunising a mammal (such as a mouse or a rabbit) with a protein of the invention and obtaining polyclonal antibodies or monoclonal antibodies by conventional techniques. For example, polyclonal antisera may be obtained by bleeding the immunized animal into a glass or plastic container, incubating the blood at 25° C. for one hour, followed by incubating at 4° C. for 2-18 hours. The serum is recovered by centrifugation (e.g. 1,000 g for 10 minutes). Monoclonal antibodies may be prepared using the standard method of Kohler & Milstein [Nature (1975) 256:495-96], or a modification thereof, or by any other suitable method.

### Immunogenic Compositions and Medicaments

The protein, antibody, and/or nucleic acid or medicament may be in the form of composition. These compositions may be suitable as immunogenic compositions (e.g. vaccines), or as diagnostic reagents.

It is particularly advantageous to use a protein of the invention in an immunogenic composition such as a vaccine. Preferably, the final formulation of the vaccine is more stable compared with immunogenic compositions that comprise wild-type Chlamydia HtrA. It is also envisaged that the immunogenic composition may comprise a nucleic acid which encodes a protein of the invention such that the protein is generated in vivo.

An immunogenic composition of the invention comprises a protein, antibody and/or nucleic acid according to the invention. Immunogenic compositions according to the invention may either be prophylactic (i.e. to prevent infection) or therapeutic (i.e. to treat infection), but will typically be prophylactic. Where the immunogenic composition is for prophylactic use, the human is preferably a child (e.g. a toddler or infant) or a teenager; where the immunogenic composition is for therapeutic use, the human is preferably a teenager or an adult. An immunogenic composition intended for children may also be administered to adults e.g. to assess safety, dosage, immunogenicity, etc.

In some embodiments, the immunogenic composition is for treatment or prevention of Chlamydia infection or an associated condition (e.g. trachoma, blindness, cervicitis, pelvic inflammatory disease, infertility, ectopic pregnancy, chronic pelvic pain, salpingitis, urethritis, epididymitis, infant pneumonia, patients infected with cervical squamous cell carcinoma, and/or HIV infection, etc.), preferably, C. trachomatis infection. The immunogenic composition may be effective against C. pneumoniae.

Immunogenic compositions used as vaccines comprise an immunologically effective amount of the protein of the invention, as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of the individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

Antigens in the composition will typically be present at a concentration of at least 1 μg/ml each.

In general, the concentration of any given antigen will be sufficient to elicit an immune response against that antigen.

Dosage treatment can be a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.).

The pH of an immunogenic composition is preferably between 6 and 8, preferably about 7. pH may be maintained by the use of a buffer. The composition may be sterile and/or pyrogen-free. The composition may be isotonic with respect to humans.

Immunogenic compositions of the invention will generally be administered directly to a patient. Direct delivery may be accomplished by parenteral injection (e.g. subcutaneously, intraperitoneally, intravenously, intramuscularly, or to the interstitial space of a tissue), or mucosally, such as by rectal, oral (e.g. tablet, spray), vaginal, topical, transdermal (See e.g. WO99/27961) or transcutaneous (See e.g. WO02/074244 and WO02/064162), intranasal (See e.g. WO03/028760), ocular, aural, pulmonary or other mucosal administration.

Chlamydia infections affect various areas of the body and so the immunogenic compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition). The composition may be prepared for topical administration e.g. as an ointment, cream or powder. The composition may be prepared for oral administration e.g. as a tablet or capsule, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops.

The invention also provides a delivery device pre-filled with an immunogenic composition of the invention.

The invention also provides a kit comprising a first component and a second component wherein neither the first component nor the second component is a composition of the invention as described herein, but wherein the first component and the second component can be combined to provide a composition of the invention as described herein. The kit may further include a third component comprising one or more of the following: instructions, syringe or other delivery device, adjuvant, or pharmaceutically acceptable formulating solution.

Combinations With Other Antigens The immunogenicity of other known Chlamydia antigens may be improved by combination with a protein of the invention. The invention thus includes an immunogenic composition comprising a combination of Chlamydia antigens, said combination comprising a protein of the invention in combination with one or more additional Chlamydia antigens. Also provided is a protein or nucleic acid of the invention for a use as described above, wherein the protein or nucleic acid is for use in combination with one or more additional Chlamydia antigens (or their encoding nucleic acids). The one or more additional antigens (e.g. 2, 3, 4, 5, 6, 7 or more additional antigens) may be administered simultaneously, separately or sequentially with the protein of the invention, for example as a combined preparation.

Likewise, the antibodies of the invention may be used in combination with one or more antibodies specific for one or more additional Chlamydia antigens for use in diagnosis of Chlamydia infections.

Preferably, the one or more additional Chlamydia antigens are susceptible to proteolysis by wild-type Chlamydia HtrA. The use of the protein of the invention instead of wild-type HtrA advantageously Overcomes this problem as its protease activity has been reduced or eliminated.

In one embodiment, the one of more additional Chlamydia antigens are selected from the antigens presented in Table 2. For example, one or more (for example, all) of the additional antigens are selected from the Chlamydia trachomatis antigens listed in Table 2, but may alternatively or additionally be selected from the Chlamydia pneumoniae antigens listed in Table 2. In one embodiment, one or more of the one or more additional antigens are selected from CT372, CT443, CT043, CT153, CT279, CT601, CT711, CT114, CT480, CT456, CT381, CT089, CT734 and CT016. These additional antigens are listed in Table 2 and their sequences are set out in the “Sequences” section that follows Table 2. In one embodiment, a protein of the invention is combined with CT089. In another embodiment, a protein of the invention is combined with CT089 and CT381. Preferred combinations are a protein of the invention with one or more antigens selected from CT372, CT443, CT601, CT153 and CT279. Another preferred combination includes a HtrA mutant of the invention in combination with 1, 2 or 3 of CT456, CT733 and/or CT043 (in particular a combination of all four antigens). Advantageous combinations of the invention are those in which two or more antigens act synergistically. Thus, the protection against Chlamydia achieved by their combined administration exceeds that expected by mere addition of their individual protective efficacy.

The one or more additional Chlamydia antigens may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to a sequence presented in Table 2; and/or (b) comprising a fragment of at least ‘n’ consecutive amino acids of a sequence presented in Table 2, wherein is 7 or more (e.g. 8, 10, 12, 14, 16, 18, 20, 25, 30, 35, 40, 50, 60, 70, 80, 90, 100, 150, 200, 250 or more). These one or more additional Chlamydia antigens include variants of a sequence presented in Table 2. Preferred fragments of (b) comprise an epitope from a sequence presented in Table 2. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6. 9, 10, 15, 20, 25 or more) from the N-terminus of a sequence presented in Table 2, while retaining at least one epitope of a sequence presented in Table 2. Other fragments omit one or more protein domains. When an additional Chlamydia antigen comprises a sequence that is not identical to a complete sequence from Table 2 (e.g. when it comprises a sequence with less than 100% sequence identity thereto, or when it comprises a fragment thereof), it is preferred in each individual instance that the additional Chlamydia antigen can elicit an antibody that recognises a protein having the complete sequence from the Table 2 antigen from which it is derived.

The invention also provides a kit comprising a protein of the invention and one or more additional antigens for simultaneous, separate or sequential administration.

The Chlamydia antigens used in the invention may be present in the composition as individual separate polypeptides. Alternatively, the combination may be present as a hybrid polypeptide in which two or more (i.e. 2. 3, 4, 5, 6, 7, 8, 9, 10, 11. 12, 13, 14, 15. 16, 17, 18, 19 or 20 or more) of the antigens are expressed as a single polypeptide chain. Hybrid polypeptides offer two principal advantages: first, a polypeptide that may be unstable or poorly expressed on its own can be assisted by adding a suitable hybrid partner that overcomes the problem; second, commercial manufacture is simplified as only one expression and purification need be employed in order to produce two polypeptides which are both antigenically useful. Different hybrid polypeptides may be mixed together in a single formulation. Within such combinations, a Chlamydia trachomatis antigen may be present in more than one hybrid polypeptide and/or as a non-hybrid polypeptide. It is preferred, however, that an antigen is present either as a hybrid or as a non-hybrid, but not as both.

Hybrid polypeptides can be represented by the formula NH₂—A—{—X—L—}ₙ—B—COOH, wherein: at least one X is an amino acid sequence of a Chlamydia HtrA protein according to the invention as described above; L is an optional linker amino acid sequence; A is an optional N-terminal amino acid sequence; B is an optional C-terminal amino acid sequence; n is an integer of 2 or more (e.g. 2, 3, 4, 5, 6, etc.). Usually n is 2 or 3.

If a —X— moiety has a leader peptide sequence in its wild-type form, this may be included or omitted in the hybrid protein. In some embodiments, the leader peptides will be deleted except for that of the —X— moiety located at the N-terminus of the hybrid protein i.e. the leader peptide of X₁ will be retained, but the leader peptides of X₂ . . . Xₙ will be omitted. This is equivalent to deleting all leader peptides and using the leader peptide of X₁ as moiety —A—.

For each a instances of {—X—L—}, linker amino acid sequence —L— may be present or absent. For instance, when n=2 the hybrid may be NH₂—X₁—L₁—X₂—L₂—COOH, NH₂—X₁—L₁—X₂—COOH, NH₂—X₁—X₂—L₂—COOH, etc. Linker amino acid sequence(s) —L— will typically be short (e.g. 20 or fewer amino acids i.e. 20, 19, 18, 17, 16, 15, 14, 13, 12, 11, 10, 9, 8, 7, 6, 5, 4, 3, 2, 1). Examples short peptide sequences which facilitate cloning, poly-glycine linkers(i.e. comprising Gly, where n2, 3, 4, 5, 6, 7, 8, 9, 10 or more), and histidine tags (i.e. His where n=3, 4, 5, 6, 7, 8, 9, 10 or more, Other suitable linker amino acid sequences will be apparent to those skilled in the art. A useful linker is GSGGGG (SEQ ID NO:69), with the Gly-Ser dipeptide being formed from a BamHI restriction site, thus aiding cloning and manipulation, and the (Gly₄ tetrapeptide being a typical poly-glycine tinker.

—A— is an optional N-terminal amino acid sequence. This will typically be short (e.g. 40 or fewer amino acids i.e. 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. histidine tags i.e. His, where n=3, 4, 5, 6, 7, 8, 9, 10 or more). Other suitable N-terminal amino acid sequences will be apparent to those skilled in the art. If X₁ lacks its own N-terminus methionine, —A— is preferably an oligopeptide (e.g. with 1, 2, 3, 4, 5, 6, 7 or 8 amino acids) which provides a N-terminus methionine.

—B— is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. Hisₙ where n=3, 4, 5, 6, 7, 8, 9, 10 or more), or sequences which enhance protein stability. Other suitable C-terminal amino acid sequences will be apparent to those skilled in the art.

Where hybrid polypeptides are used, the individual antigens within the hybrid (i.e. individual —X— moieties) may be from one or more strains. Where n=2, for instance, X₂ may be from the same strain as X₁ or from a different strain. Where n=3, the strains might be (i) X₁X₂=X₃ (ii) X_{1=X}₂ [symbol] X₃ (iii) X₁ [symbol] X₂=X₃ (iv) X₁ [symbol] X₂ [symbol] X₃ or (v) X₁=X₃ [symbol] X_{2,} etc.

The invention also provides a nucleic acid encoding a hybrid polypeptide of the invention. Furthermore, the invention provides a nucleic acid which can hybridise to this nucleic acid, preferably under “high stringency” conditions (e.g. 65° C. in a 0.1×SSC, 0.5% SDS solution).

### Further Components of the Composition

Compositions may thus be pharmaceutically acceptable. They will usually include components in addition to the antigens e.g. they typically include one or more pharmaceutical carrier(s) and/or excipient(s). A thorough discussion of such components is available in Remington The Science and Practice of Pharmacy.

Compositions will generally be administered to a mammal in aqueous form. Prior to administration, however, the composition may have been in a non-aqueous form, For instance, although some vaccines are manufactured in aqueous form, then filled and distributed and administered also in aqueous form, other vaccines are lyophilised during manufacture and are reconstituted into an aqueous form at the time of use. Thus a composition of the invention may be dried, such as a lyophilised formulation.

The composition may include preservatives such as thiomersal or 2-phenoxyethanol. It is preferred, however, that the vaccine should be substantially free from (i.e. less than 5 μg/ml) mercurial material e.g. thiomersal-free. Vaccines containing no mercury are more preferred. Preservative-free vaccines are particularly preferred.

To control tonicity, it is preferred to include a physiological salt, such as a sodium salt. Sodium chloride NaCl) is preferred, which may be present at between 1 and 20 mg/ml e.g. about 10±2 mg/ml NaCl. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, preferably between 240-360 mOsm/kg, and will snore preferably fall within the range of 290-310 mOsm/kg.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a composition will generally be between 5.0 and 8.1, and more typically between 6.0 and 8.0 e.g. 6.5 and 7.5, or between 7.0 and 7.8.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunisation, or may include material for multiple immunisations (i.e. a multidose kit). The inclusion of a preservative is preferred in multidose arrangements. As an alternative (or in addition) to including a preservative in multidose compositions, the compositions may be contained in a container having an aseptic adaptor for removal of material.

Human vaccines are typically administered in a dosage volume of about 0.5 ml, although a half dose (i.e. about 0.25 ml) may be administered to children.

Immunogenic compositions of the invention may also comprise one or more immunoregulatory agents. Preferably, one or more of the immunoregulatory agents include one or more adjuvants. The adjuvants may include a TH1 adjuvant and/or a TH2 adjuvant, further discussed below.

Adjuvants which may be used in compositions of the invention include, but are not limited to:

### A. Mineral-Containing Compositions

Mineral containing compositions suitable for use as adjuvants in the invention include mineral salts, such as aluminium salts and calcium salts (or mixtures thereof). Calcium salts include calcium phosphate (e.g. the “CAP” particles disclosed in U.S. Pat. No. 6,355,271). Aluminum salts include hydroxides, phosphates, sulfates, etc., with the salts taking any suitable form (e.g. gel, crystalline, amorphous, etc.). Adsorption to these salts is preferred. The mineral containing compositions may also be formulated as a particle of metal salt [WO00/23105].

The adjuvants known as aluminum hydroxide and aluminum phosphate may be used. These names are conventional, but are used for convenience only, as neither is a precise description of the actual chemical compound which is present (e.g. see chapter 9 of Vaccine Design . . . (1995) eds. Powell & Newman, ISBN: 030644867X, Plenum). The invention can use any of the “hydroxide” or “phosphate” adjuvants that are in general use as adjuvants. The adjuvants known as “aluminium hydroxide” are typically aluminium oxyhydroxide salts, which are usually at least partially crystalline. The adjuvants known as “aluminium phosphate” are typically aluminium hydroxyphosphates, often also containing a small amount of sulfate (i.e. aluminium hydroxyphosphate sulfate). They may be obtained by precipitation, and the reaction conditions and concentrations during precipitation influence the degree of substitution of phosphate for hydroxyl in the salt.

A fibrous morphology (e.g. as seen in transmission electron micrographs) is typical for aluminium hydroxide adjuvants. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

Aluminium phosphate adjuvants generally have a PO₄/Al molar ratio between 0.3 and 1.2, preferably between 0.8 and 1.2, and more preferably 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate (e.g. plate-like morphology as seen in transmission electron micrographs). Typical diameters of the particles are in the range 0.5-20 μm (e.g. about 5-10 μm) after any antigen adsorption. Adsorptive capacities of between 0.7-1.5 mg protein per mg Al⁺⁺⁺ at pH 7.4 have been reported for aluminium phosphate adjuvants.

The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

Suspensions of aluminium salts used to prepare compositions of the invention may contain a buffer (e.g. a phosphate or a histidine or a Tris buffer), but this is not always necessary. The suspensions are preferably sterile and pyrogen-free. A suspension may include free aqueous phosphate ions e.g. present at a concentration between 1.0 and 20 mM, preferably between 5 and 15 mM, and more preferably about 10 mM. The suspensions may also comprise sodium chloride.

The invention can use a mixture of both an aluminium hydroxide and an aluminium phosphate this case there may be more aluminium phosphate than hydroxide e.g. a weight ratio of at least 2:1 e.g. ≧5:1, ≧6:1, ≧7:1, ≧8:1, ≧9:1, etc.

The concentration of Al⁺⁺⁺⁺ in a composition far administration to a patient is preferably less than 10 mg/ml e.g. ≦5 mg/ml, ≦4 mg/ml, ≦3 mg/ml, ≦2 mg/ml, ≦1 mg/ml, etc. A preferred range is between 0.3 and 1 mg/ml. A maximum of 0.85 mg/dose is preferred.

Aluminium phosphates are particularly preferred, particularly in compositions which include a H. influenzae saccharide antigen, and a typical adjuvant is amorphous aluminium hydroxyphosphate with PO4/Al molar ratio between 0.84 and 0.92, included at 0.6 mg Al³⁺/ml. Adsorption with a low dose of aluminium phosphate may be used e.g. between 50 and 100 μg Al³⁺ per conjugate per dose. Where there is more than one conjugate in a composition, not all conjugates need to be adsorbed.

### B. Oil Emulsions

Oil emulsion compositions suitable for use as adjuvants in the invention include squalene-water emulsions, such as MF59 [Chapter 10 of Vaccine Design . . . (1995) eds. Powell & Newman. ISBN: 030644867X. Plenum; see also WO90/14837] (5% squalene, 0.5% TWEEN80®, and 0.5% SPAN85®, formulated into submicron particles using a microfluidizer). Complete Freund's adjuvant (CFA) and incomplete Freund's adjuvant (IFA) may also be used.

Various oil-in-water emulsion adjuvants are known, and they typically include at least one oil and at least one surfactant, with the oil(s) and surfactant(s) being biodegradable (metabolisable) and biocompatible. The oil droplets in the emulsion are generally less than 5 μm in diameter, and ideally have a sub-micron diameter, with these small sizes being achieved with a microfluidiser to provide stable emulsions. Droplets with a size less than 220 mn are preferred as they can be subjected to filter sterilization.

The emulsion can comprise oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoids known as squalene, 2,6,10,15,19,23-hexamethyl-2,6,10,14,18,22-tetracosahexaene, which is particularly preferred herein. Squalene, the saturated analog to squalene, is also a preferred oil. Fish oils, including squalene and squalene, are readily available from commercial sources or may be obtained by methods known in the art. Other preferred oils are the tocopherols (see below). Mixtures of oils can be used.

Surfactants can be classified by their ‘HLB’ (hydrophile/lipophils balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, Of t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants are preferred. Preferred surfactants for including in the emulsion are Tween 80 (polyoxyethylene sorbitan monooleate), Span 85 (sorbitan trioleate), lecithin and Triton X-100.

Mixtures of surfactants can be used e.g. Tween 80/Span 85 mixtures. A combination of a polyoxyethylene sorbitan ester such as polyoxyethylene sorbitan monooleate (Tween 80) and an octoxynol such as t-octylphenoxypolyethoxyethanol (Triton X-100) is also suitable. Another useful combination comprises laureth 9 plus a polyoxyethylene sorbitan ester and/or an octoxynol.

Preferred amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Preferred emulsion adjuvants have an average droplets size of <1 μm e.g. <750 nm, <500 nm, <400 nm, <300 nm, <250 nm, <220 nm, <200 nm, or smaller. These droplet sizes can conveniently be achieved by techniques such as mierofluidisation.

Specific oil-in-water emulsion adjuvants useful with the invention include, but are not limited to:

A submicron emulsion of squalene, TWEEN80®, and SPAN85®. The composition of the emulsion by volume can be about 5% squalene, about 0.5% polysorbate 80 and about 0.5% SPAN85®. In weight terms, these ratios become 4.3% squalene, 0.5% polysorbate 80 and 0.48% SPAN85®. This adjuvant is known as ‘MF59’ (WO90/14837, Podda & Del Giudice (2003) Expert Rev Vaccines 2:197-203, Podda (2001) Vaccine 19: 2673-2680; as described in more detail in Chapter 10 of Vaccine Design: The Subunit and Adjuvant Approach (eds. Powell & Newman) Plenum Press 1995 (ISBN 0-306-44867-X) and chapter 12 of Vaccine Ajuvants: Preparation Methods and Research Protocols (Volume 42 of Methods in Molecular Medicine series). ISBN: 1-59259-083-7. Ed. O'Hagan). The MF59 emulsion advantageously includes citrate ions e.g. 10 mM sodium citrate buffer.

An emulsion of squalene, a tocopherol, and TWEEN80®. The emulsion may include phosphate buffered saline. It may also include SPAN85® (e.g. at 1%) and/or lecithin. These emulsions may have from 2 to 10% squalene, from 2 to 10% tocopherol and from 0.3 to 3% TWEEN80®, and the weight ratio of squalene:tocopherol is preferably <1 as this provides a more stable emulsion. Squalene and TWEEN80® may be present volume ratio of about 5:2. One such emulsion can be made by dissolving TWEEN80® in PBS to give a 2% solution, then mixing 90 ml of this solution with a mixture of (5 g of DL-α-tocopherol and 5 ml squalene), then microfluidising the mixture. The resulting emulsion may have submicron oil droplets e.g. with an average diameter of between 100 and 250 nm, preferably about 180 nm.

An emulsion of squalene, a tocopherol, and a Triton detergent e.g., TRITON X-100®). The emulsion may also include a 3d-MPL (see below). The emulsion may contain a phosphate buffer.

An emulsion comprising a polysorbate (e.g. polysorbate 80), a Triton detergent (e.g. TRITON X-100®) and a tocopherol (e.g. an α-tocopherol succinate). The emulsion may include these three components at a mass ratio of about 75:11:10 (e.g. 750 μg/ml polysorbate 80, 110 μg/ml TRITON X-100® and 100 μg/ml α-tocopherol succinate), and these concentrations should include any contribution of these components from antigens. The emulsion may also include squalene. The emulsion may also include a 3d-MPL (see below). The aqueous phase may contain a phosphate buffer.

An emulsion of squalene, polysorbate 80 and poloxamer 401 (“PLURONIC™ L121”). The emulsion can be formulated in phosphate buffered saline, pH 7.4. This emulsion is a useful delivery vehicle for muramyl dipeptides, and has been used with threonyl-MDP in the “SAF-1” adjuvant (Allison & Byars (1992) Res Immunol 143:519-25) (0.05-1% Thr-MDP, 5% squalene, 2.5% Pluronic L121 and 0.2% polysorbate 80). It can also be used without the Thr-MDP, as in the “AF” adjuvant (Hariharan et al. (1995) Cancer Res 55:3486-9) (5% squalene, 1.25% Pluronic 121 and 0.2% polysorbate 80). Microfluidisation is preferred.

An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. a sorbitan ester or mannide ester, such as sorbitan monoleate or ‘SPAN80®’). The emulsion is preferably thermoreversible and/or has at least 90% of the oil droplets (by volume) with a size less than 200 nm (US-2007/014805.). The emulsion may also include one or more of: alditol; a cryoprotective agent (e.g. a sugar, such as dodecylmaltoside and/or sucrose); and/or an alkylpolyglycoside. Such emulsions may be lyophilized.

An emulsion of squalene, poloxamer 105 and ABIL® Care (Suli et al. (2004) Vaccine 22(25-26):3464-9). The final concentration (weight) of these components in adjuvanted vaccines are 5% squalene, 4% poloxamer 105 (pluronic polyol) and 2% ABIL® Care 85 (Bis-PEG/PPG-16/16 PEG/PPG-16/16 dimethicone; caprylic/capric triglyceride).

An emulsion having from 0.5-50% of an oil, 0.1-10% of a phospholipid, and 0.05-5% of a non-ionic surfactant. As described in WO95/11700, preferred phospholipid components are phosphatidylcholine, phosphatidylethanolamine, phosphatidylserine, phosphatidylinositol, phosphatidylglycerol phosphatidic acid, sphingomyelin and cardiolipin. Submicron droplet sizes are advantageous.

A submicron oil-in-water emulsion of a non-metabolisable oil (such as light mineral oil) and at least one surfactant (such as lecithin, TWEEN80® or SPAN80®). Additives may be included, such as QuilA saponin, cholesterol, a saponin-lipophile conjugate (such as GPI-0100, described in U.S. Pat. No. 6,080,725, produced by addition of aliphatic amine to desacylsaponin via the carboxyl group of glucuronic acid), dimethyidioctadecylammonium bromide and/or N,N-dioctadecyl-N,N-bis (2-hydroxyethyl)propanediamine.

An emulsion in which a saponin (e.g. QuilA or QS21) and a sterol (e.g. a cholesterol) are associated as helical micelles (WO2005/097181). An emulsion comprising a mineral oil, a non-ionic lipophilic ethoxylated fatty alcohol, and a non-ionic hydrophilic surfactant (e.g. an ethoxylated fatty alcohol and/or polyoxyethylene-polyoxypropylene block copolymer) (WO2006/113373).

An emulsion comprising a mineral oil, a non-ionic hydrophilic ethoxylated fatty alcohol, and a non-ionic lipophilic surfactant (e.g. an ethoxylated fatty alcohol and/or polyoxyethylene-polyoxypropylene block copolymer) (Wu et al. (2004) Antiviral Res. 64(2):79-83).

In some embodiments an emulsion may be mixed with antigen extemporaneously, at the time of delivery, and thus the adjuvant and antigen may be kept separately in a packaged or distributed vaccine, ready for final formulation at the time of use. In other embodiments an emulsion is mixed with antigen during manufacture, and thus the composition is packaged in a liquid adjuvanted form. The antigen will generally be in an aqueous form, such that the vaccine is finally prepared by mixing two liquids. The volume ratio of the two liquids for mixing can vary (e.g. between 5:1 and 1:5) but is generally about 1:1. Where concentrations of components are given in the above descriptions of specific emulsions, these concentrations are typically for an undiluted composition, and the concentration after mixing with an antigen solution will thus decrease. Where a composition is to be prepared extemporaneously prior to use (e.g. where a component is presented in lyophilised form) and is presented as a kit, the kit may comprise two vials, or it may comprise one ready-filled syringe and one vial, with the contents of the syringe being used to reactivate the contents of the vial prior to injection.

Where a composition eludes a tocopherol, any of the α, μ, γ, δ, ε or ζ tocopherols can be used, but α-tocopherols are preferred. The tocopherol can take several forms e.g. different salts and/or isomers. Salts include organic salts, such as succinate, acetate, nicotinate, etc. D-α-tocopherol and DL-α-tocopherol can both be used. Tocopherols are advantageously included in vaccines for use in elderly patients (e.g. aged 60 years or older) because vitamin E has been reported to have a positive effect on the immune response in this patient group (Han et al. (2005) Impact of Vitamin E on Immune Function and infectious Diseases in the Aged at Nutrition, Immune functions and Health EuroConference, Paris, 9-10 Jun. 2005). They also have antioxidant properties that may help to stabilize the emulsions (U.S. Pat. No. 6,630,161). A preferred α-tocopherol is DL-α-tocopherol, and the preferred salt of this tocopherol is the succinate. The succinate salt has been found to cooperate with TNF-related ligands in vivo.

Saponin Formulations (Chapter 22 Vaccine Design . . . (1995) eds. Powell & Newman. ISBN: 030644867X. Plenum)

Saponin formulations may also be used as adjutants in the invention. Saponins are a heterogeneous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officinalis (soap root). Saponin adjuvant formulations include purified formulations, such as QS21, as well as lipid formulations, such as ISCOMs. QS21 is marketed as Stimulon™.

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (WO96/33739).

Combinations of saponins and cholesterols can be used to form unique particles called immunostimulating complexs (ISCOMs) (chapter 23 of Vaccine Design . . . (1995) eds. Powell & Newman. ISBN: 030644867X, Plenum). ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in Podda & Del Giudice (2003) Expert Rev Vaccines 2:197-203; Podda (2001) Vaccine 19: 2673-2680; Vaccine Design: The Subunit and Adjuvant Approach (eds. Powell & Newman) Plenum Press 1995 (ISBN 0-306-44867-X); Vaccine Adjuvants: Preparation Methods and Research Protocols (Volume 42 of Methods in Molecular Medicine series). ISBN: 1-59259-083-7. Ed. O'Hagan; Allison & Byars (1992) Res Immunol 143:519-25; Hariharan et al. (1995) Cancer Res 55:3486-9; US-2007/014805; Suli et al. (2004) Vaccine 22(25-26):3464-9; WO95/11700; U.S. Pat. No. 6,080,725; WO2005/097181; WO2006/113373: Han et al. (2005) Impact of Vitamin E on Immune Function and Infectious Diseases in the Aged at Nutrition, Immune functions and Health EuroConference, Paris, 9-10 Jun. 2005; U.S. Pat. No. 6,630,161; U.S. Pat. No. 5,057,540; WO96/33739; EP-A-0109942; and WO96/11711. Optionally, the ISCOMS may be devoid of additional detergent (WO00/07621).

A review of the development of saponin based adjuvants can be found in Barr et al. (1998) Advanced Drug Delivery Reviews 32:247-271 and Sjolanderet al. (1998) Advanced Drug Delivery Reviews 32:321-338.

### D. Virosomes and Virus-Like Particles

Virosomes and virus-like particles (VPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in Niikura et al. (2002) Virology 293:273-280; Lenz et al. (2001) J Immunol 166:5346-5355; Pinto et al. (2003) J Infect Dis 188:327-338; Gerber et at. (2001) J Virol 75:4752-4760; WO03/024480 and WO03/024481. Virosomes are discussed further in, for example, Gluck et al. (2002) Vaccine 20:B10-B16.

### E. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof. Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in EP-A-0689454. Such “small particles” of 3dMPL are small enough to be sterile filtered through a 0.22 μm membrane (U.S. Pat. No. 6,630,161). Other non-toxic LPS derivatives include monophosphoryl lipid A mimics, such as aminoalkyl glucosaminide phosphate derivatives e.g. RC-529 (Johnson et al. (1999) Bioorg Med Chem Lett 9:2273-2278; and Evans et al. (2003) Expert Rev Vaccines 2:219-229). Lipid A derivatives include derivatives of lipid A from Escherichia coli such as OM-174. OM-174 is described for example in Meraldi et al. (2003) Vaccine 21:2485-2491 and Pajak et al. (2003) Vaccine 21:836-842.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. Kandimalla et al. (2003) Nucleic Acids Research 31:2393-2400, WO02/26757 and WO99/62923 disclose possible analog substitutions e.g. replacement of guanosine with 2′-deoxy-7-deazaguanosine. The adjuvant effect of CpG oligonucleotides is further discussed in Krieg (2003) Nature Medicine 9:831-835; McCluskie et al. (2002) FEMS Immunology and Medical Microbiology 32:179-185; WO98/40100; U.S. Pat. No. 6,207,646; U.S. Pat. No. 6,239,116 and U.S. Pat. No. 6,429,199.

The CpG sequence may be directed to TLR9, such as the motif GTCGTT or TTCGTT (Kandimalla et al. (2003) Biochemical Society Transactions 31 (part 3):654-658). The CpG sequence may be specific for inducing a Th1 immune response, such as a CpG-A ODN, or it may be more specific for inducing a B cell response, such a CpG-B ODN. CpG-A and CpG-B ODNs are discussed in Blackwell et al. (2003) J Immunol 170:4061-4068; Krieg (2002) Trends Immunol 23:64-65; and WO01/95935. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, Gluck et al. (2002) Vaccine 20:B10-B16; Kandimalla et al. (2003) BBRC 306:948-953; Bhagat et al. (2003) BBRC 300:853-861; and WO03/035836.

A used CpG adjuvant is CpG7909, also known as ProMune™ (Coley Pharmaceutical Group, Inc.). Another is CpG1826. As an alternative, or in addition, to using CpG sequences, TpG sequences can be used (WO01/22972), and these oligonucleotides may be free from unmethylated CpG motifs. The immunostimulatory oligonucleotide may be pyrimidine-rich. For example, it may comprise more than one consecutive thymidine nucleotide (e.g. TTTT, as disclosed in Pajak et al. (2003) Vaccine 21:836-842), and/or it may have a nucleotide composition with >25% thymidine (e.g. >35%, >40%, >50%, >60%, >80%, etc.). For example, it may comprise more than one consecutive cytosine nucleotide (e.g. CCCC, as disclosed in Pajak et al. (2003) Vaccine 21:836-842), and/or it may have a nucleotide composition with >25% cytosine (e.g. >35%, >40%, >50%, >60%, >80%, etc.). These oligonucleotides may be free from unmethylated CpG motifs. Immunostimulatory oligonucleotides will typically comprise at least 20 nucleotides. They may comprise fewer than 100 nucleotides.

A particularly useful adjuvant based around immunostimulatory oligonucleotides is known as IC-31™ (Schellack et al. (2006) Vaccine 24:5461-72). Thus an adjuvant used with the invention may comprise a mixture of (i) an oligonucleotide (e.g. between 15-40 nucleotides) including at least one (and preferably multiple) Cp1 motifs (i.e. a cytosine linked to an inosine to form a dinucleotide), and (ii) a polycationic polymer, such as an oligopeptide (e.g. between 5-20 amino acids) including at least one (and preferably multiple) Lys-Arg-Lys tripeptide sequence(s). The oligonucleotide may be a deoxynucleotide comprising 26-mer sequence 5′-(IC)₁₃-3′. The polycationic polymer may be a peptide comprising 11-mer amino acid sequence KLKLLLLLKLK (SEQ ID NO:70).

Bacterial ADP-ribosylating toxins and detoxified derivatives thereof may be used as adjuvants in the invention. Preferably, the protein is derived from E. coli (E. coli heat labile enterotoxin “LT”), cholera (“CT”), or pertussis (“PT”). The use of detoxified ADP-ribosylating toxins as mucosal adjuvants is described in WO95/17211 and as parenteral adjuvants in WO98/42375. The toxin or toxoid is preferably in the form of a hole toxin, comprising both A and B subunits. Preferably, the A subunit contains a detoxifying mutation; preferably the B subunit is not mutated. Preferably, the adjuvant is a detoxified LI mutant such as LT-K63, LT-R72, and LT-G192. The use of ADP-ribosylating toxins and detoxified derivatives thereof, particularly LT-K63 and LT-R72, as adjuvants can be found in Beignon et al. (2002) Infect Immun 70:3012-3019; Pizza et al. (2001) Vaccine 19:2534-2541; Pizza et al. (2000) Int J Med Microbiol 290:455-461; Scharton-Kersten et al, (2000) Infect Immun 68:5306-5313: Ryan et al. (1999) Infect Immun 67:6270-6280: Partidos et al. (1999) Immunol Lett 67:209-216; Peppoloni et al. (2003) Expert Rev Vaccines 2:285-293; and Pine et al. (2002) J Control Release 85:263-270.

A useful CT mutant is or CT-E29H (Tebbey et al. (2000) Vaccine 18:2723-34). Numerical reference for amino acid substitutions is preferably based on the alignments of the A and B subunits of ADP-ribosylating toxins set forth in Domenighini et al. (1995) Mol Microbiol 15:1165-1167, specifically incorporated herein by reference in its entirety.

### F. Human Immunomodulators

Human immunomodulators suitable for use as adjuvants in the invention include cytokines, such as interleukins (e.g. IL-1, IL-2, IL-4, IL-5, IL-6, IL-7, IL-12 (WO99/40936), etc.) (WO99/44636), interferons (e.g. interferon-65 ), macrophage colony stimulating factor, and tumor necrosis factor. A preferred immunomodulator is IL-12.

### G. Bioadhesives and Mucoadhesives

Bioadhesives and mucoadhesives may also be used as adjuvants in the invention. Suitable bioadhesives include esterified hyaluronic acid microspheres (Singh et al. (2001) J Cont Release 70:267-276) or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention (WO99/27960).

### H. Microparticles

Microparticles may also be used as adjuvants in the invention. Microparticles (i.e. a particle of ˜100 nm to ˜150 nm in diameter, more preferably ˜200 mn to ˜30 μm in diameter, and most preferably ˜500 mn to ˜10 nm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

I. Liposomes (Chapters 13 & 14 of Vaccine Design . . . (1995) eds. Powell & Newman. ISBN: 030644867X Plenum)

Examples of liposome formulations suitable for use as adjuvants are described in U.S. Pat. No. 6,090,406; U.S. Pat. No. 5,916,588; and EP-A-0626169.

### J. Polyoxyethylene Ether and Polyoxyethylene Ester Formulations

Adjuvants suitable for use in the invention include polyoxyethylene ethers and polyoxyethylene esters (WO99/52549). Such formulations further include polyoxyethylene sorbitan ester surfactants in combination with an octoxynol (WO01/21207) as well as polyoxyethylene alkyl ethers or ester surfactants in combination with at least one additional non-ionic surfactant such as an octoxynol (WO01/21152). Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### K. Phosphazenes

A phosphazene, such as poly[di(carboxylatophenoxy)phosphazene] (“PCPP”) as described, for example, in Aridrianov et al. (1998) Biomaterials 19:109-115 and Payne et al. (1998) Adv Drug Delivery Review 31:185-196, may be used.

### L. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

### M. Imidazoquinolone Compounds.

Examples of imidazoquinolone compounds suitable for use adjuvants in the invention include Imiquimod (“R-837”) (U.S. Pat. No. 4,680,338; U.S. Pat. No. 4,988,815), Resiquimod (“R-848”) (WO92/15582), and their analogs; and salts thereof (e.g. the hydrochloride salts). Further details about immunostimulatory imidazoquinolines can be found in Stanley (2002) Clin Exp Dermatol 27:571-577; Wu et al. (2004) Antiviral Res. 64(2):79-83; Vasilakos et al. (2000) Cell Immunol. 204(1):64-74; U.S. Pat. Nos. 4,689,338, 4,929,624, 5,238,944, 5,266,575, 5,268,376, 5,346,905, 5,352,784, 5,389,640, 5,395,937, 5,482,936, 5,494,916, 5,525,612, 6,083,505, 6,440,992, 6,627,640, 6,656,938, 6,660,735, 6,660,747, 6,664,260, 6,664,264, 6,664,265, 6,667,312, 6,670,372, 6,677,347, 6,677,348, 6,677,349, 6,683,088, 6,703,402, 6,743,920, 6,800,624, 6,809,203, 6,888,000 and 6,924,293; and Jones (2003) Curr Opin Investig Drugs 4:214-218.

### N. Substituted Ureas

Substituted ureas useful as adjuvants include compounds of formula I, II or III, or salts thereof:

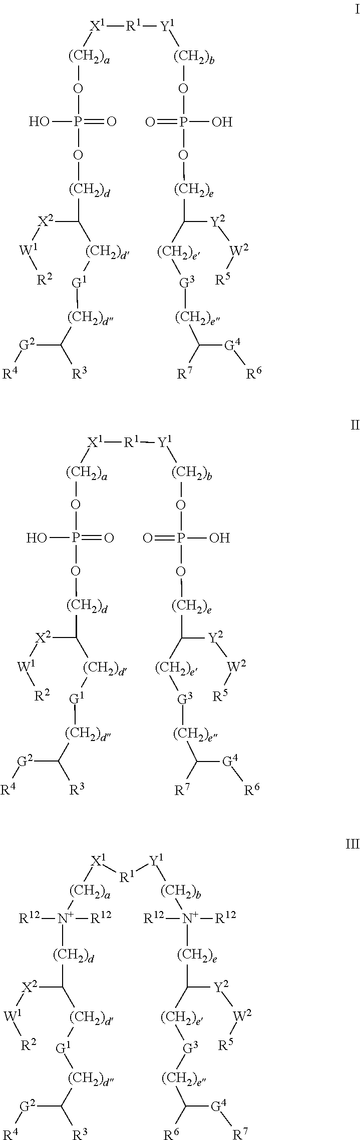

as defined in WO03/011223, such as ‘ER 803058’, ‘ER 803732’, ‘ER 804053’, ER 804058’, ‘ER 804059’, ‘ER 804442’, ‘ER 804680’, ‘ER 804764’, ER 803022 or ‘ER 804057’ e.g.

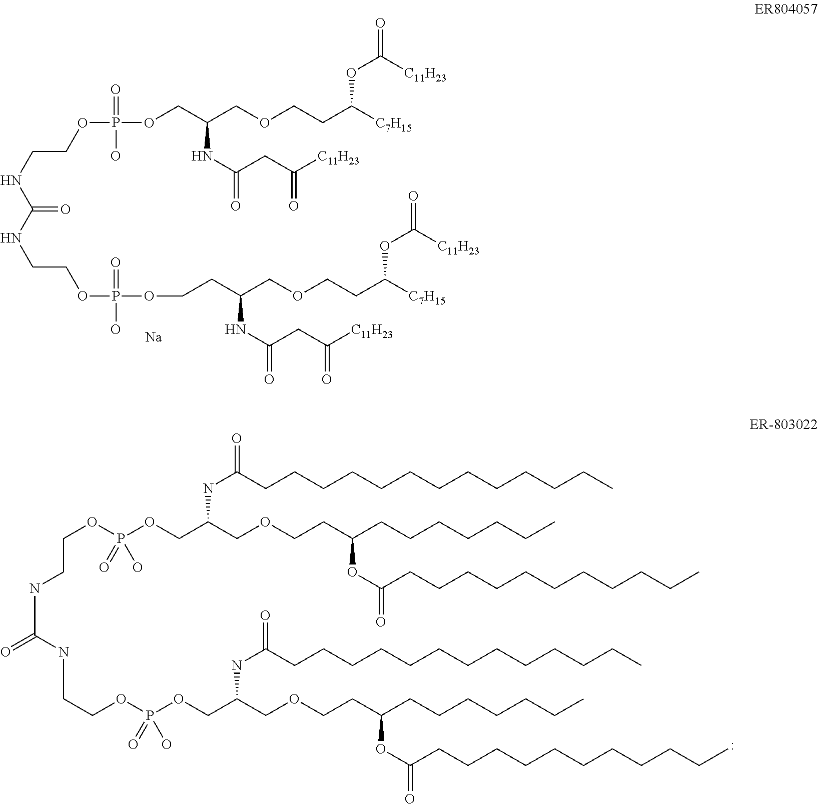

### O. Further Adjuvants

Further adjuvants that may be used with the invention include:

Am aminoalkyl glucosaminide phosphate derivative, such as RC-529 (Johnson et at. (1999) Bioorg Med Chem Lett 9:2273-2278; Evans et al. (2003) Expert Rev Vaccines 2:219-229).

A thiosemicarbazone compound, such as those disclosed in WO2004/060308. Methods of formulating, manufacturing, and screening for active compounds are also described in Bhagat et al. (2003) BBRC 300:853-861. The thiosemicarbazones are particularly effective in the stimulation of human peripheral blood mononuclear cells for the production of cytokines, such as TNF-α.

A tryptanthrin compound, such as those disclosed in WO2004/064759. Methods of formulating, manufacturing, and screening for active compounds are also described in WO03/035836. The thiosemicarbazones are particularly effective in the stimulation of human peripheral blood mononuclear cells for the production of cytokines, such as TNF-α.

A nucleoside analog, such as: (a) Isatorabine (ANA-245; 7-thia-8-oxopanosine):

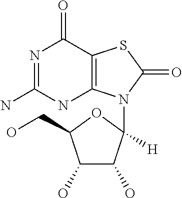 [SMILES: Nc1nc(=O)c2sc(=O)n([C@@H]3O[C@H](CO)[C@@H](O)[C@H]3O)c2[nH]1]

and prodrugs thereof; (b) ANA975; (c) ANA-025-1; (d) ANA380; (e) the compounds disclosed in U.S. Pat. No. 6,924,271, US2005/0070556 and U.S. Pat. No. 5,658,731, oxoribine (7-allyl-8-oxoguanosine) (U.S. Pat. No. 5,011,828).

Compounds disclosed in WO2004/87153, including: Acylpiperazine compounds, Indoledione compounds, Tetrahydraisoquinoline (THIQ) compounds, Benzocyclodione compounds, Aminoazavinyl compounds, Aminobenzimidazole quinolinone (ABIQ) compounds (U.S. Pat. No. 6,605,617. WO02/18383), Hydrapthalamide compounds, Benzophenone compounds, Isoxazole compounds, Sterol compounds, Quinazilinone compounds, Pyrrole compounds (WO2004/018455), Anthraquinone compounds, Quinoxaline compounds, Triazine compounds, Pyrazalopyrimidine compounds, and Benzazole compounds (WO03/082272).

Compounds containing lipids linked to a phosphate-containing acyclic backbone, such as the TLR4 antagonist E5564 (Wong et cal (2003) J Clin Pharmacol 43(7):735-42; US2005/0215517).

A polyoxidonium polymer (Dyakonova et al. (2004) Int Immunopharmacol 4(13):1615-23; FR-2859633) or other N-oxidized polyethylene-piperazine derivative.

Methyl inosine 5′-monophosphate (“MIMP”) (Signorelli & Hadden (2003) Int Immunopharmacol 3(8):1177-86).

A polyhydroxlated pyrrolizidine compound (WO2004/064715), such as one having formula:

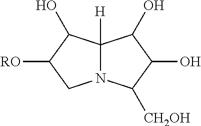

where R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof. Examples include, but are not limited to: casuarine, casuarine-6-α-D-glucopyranose, 3-epi-casuarine, 7-epi-casuarine, 3,7-diepi-casuarine, etc.

A CD1d ligand, such as an α-glycosylceramide (De Libero et al, Nature Reviews Immunology, 2005, 5: 485-496; U.S. Pat. No. 5,936,076 ; Oki et al, J. Clin. Investig., 113: 1631-1640 ; US2005/0192248; Yang et al, Angew. Chem. Int. Ed., 2004, 43: 3818-3822; WO2005/102049; Goff et al, J. Am. Chem., Soc., 2004, 126: 13602-13603; WO03/105769) e.g. α-galactosylceramide), phytosphingosine-containing α-glycosylceramides, OCH, KRN7000 [(2S,3S,4R)-1-O-(α-D-galactopyranosyl)-2-(N-hexacosanoylamino)-1,3,4-octadecanetriol], CRONY-101, 3″-O-sulfo-galactosylceramide, etc.

A gamma inulin (Cooper (1995) Pharm Biotechnol 6:559-80) or derivative thereof, such as algammulin.

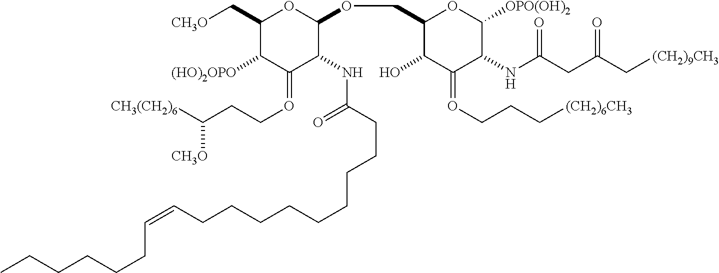

### Adjuvant Combinations

The invention may also comprise combinations of aspects of one or more of the adjuvants identified above. For example, the following adjuvant compositions may be used in the invention: (1) a saponin and an oil-in-water emulsion (WO99/11241); (2) a saponin (e.g. QS21)+a non-toxic LPS derivative (e.g. 3dMPL) (WO94/00153); (3) a saponin (e.g. QS21)+a nun-toxic LPS derivative (e.g. 3dMPL)+a cholesterol; (4) a saponin (e.g. QS21)+3dMPL+IL-12 (optionally+a sterol) (WO98/57659); (5) combinations of 3dMPL with, for example, QS21 and/or oil-in-water emulsions European patent applications 0835318, 0735898 and 0761231); (6) SAF, containing 10% squalene, 0.4% TWEEN80®, 5% pluronic-block polymer L121, and thr-MDP, either microfiuidized into a submicron emulsion or vortexed to generate a larger particle size emulsion. (7) RIBI™ adjuvant system (RAS), (Ribi Immunochem) containing 2% squalene, 0.2% TWEEN80®, and one or more bacterial cell wall components from the group consisting of monophosphorylipid A (MPL), trehalose dimycolate (TDM), and cell wall skeleton (CWS), preferably MPL+CWS (DETOX™); and (8) one or more mineral salts (such as an aluminum salt)+a non-toxic derivative of LPS (such as 3dMPL).

Other substances that act as immunostimulating agents are disclosed in chapter 7 of Vaccine Design, (1995) eds. Powell & Newman. ISBN: 030644867X. Plenum.

The use of an aluminium hydroxide and/or aluminium phosphate adjuvant is particularly preferred, and antigens are generally adsorbed to these salts. Calcium phosphate is another preferred adjuvant. Other preferred adjuvant combinations include combinations of Th1 and Th2 adjuvants such as CpG & alum or resiquimod & alum. A combination of aluminium phosphate and 3dMPL may be used.

To improve thermal stability, a composition may include a temperature protective agent. This component may be particularly useful in adjuvanted compositions (particularly those containing a mineral adjuvant, such as an aluminium salt). As described in WO2006/110603, a liquid temperature protective agent may be added to an aqueous vaccine composition to lower its freezing point e.g. to reduce the freezing point to below 0° C. Thus the composition can be stored below 0° C., but above its freezing point, to inhibit thermal breakdown. The temperature protective agent also permits freezing of the composition while protecting mineral salt adjuvants against agglomeration or sedimentation after freezing and thawing, and may also protect the composition at elevated temperatures e.g. above 40° C. A starting aqueous vaccine and the liquid temperature protective agent may be mixed such that the liquid temperature protective agent forms from 1-80% by volume of the final mixture. Suitable temperature protective agents should be safe for human administration, readily miscible/soluble in water, and should not damage other components (e.g. antigen and adjuvant) in the composition. Examples include glycerin, propylene glycol, and/or polyethylene glycol (PEG). Suitable PEGs may have an average molecular weight ranging from 200-20,000 Da. In a preferred embodiment, the polyethylene glycol can have an average molecular weight of about 300 Da (‘PEG-300’).

The invention provides an immunogenic composition comprising: (i) one or more proteins of the invention; and (ii) a temperature protective agent. This composition may be formed by mixing (i) an aqueous composition comprising one or more proteins of the invention, with (ii) a temperature protective agent. The mixture may then be stored e.g. below 0° C., from 0-20° C., from 20-35° C., from 35-55° C., or higher. It may be stored in liquid or frozen form. The mixture may be lyophilised. The composition may alternatively be formed by mixing (i) a dried composition comprising one or more proteins of the invention, with (ii) a liquid composition comprising the temperature protective agent. Thus component (ii) can be used to reconstitute component (i).

The compositions of the invention may elicit either or both of a cell mediated immune response and a humoral immune response. This immune response will preferably induce long lasting (e.g. neutralising) antibodies and a cell mediated immunity that can quickly respond upon exposure to chlamydia.

Two types of T cells, CD4 and CD8 cells, are generally thought necessary to initiate and/or enhance cell mediated immunity and humoral immunity. CD8 T cells can express a CD8 co-receptor and are commonly referred to as Cytotoxic T lymphocytes (CTLs), CD8 T cells are able to recognized or interact with antigens displayed on MHC Class I molecules.

CD4 T cells can express a CD4 co-receptor and are commonly referred to as T helper cells. CD4 T cells are able to recognize antigenic peptides bound to MHC class II molecules. Upon interaction with a MHC class II molecule, the CD4 cells can secrete factors such as cytokines. These secreted cytokines can activate B cells, cytotoxic T cells, macrophages, and other cells that participate in an immune response. Helper T cells or CD4+ cells can be further divided into two functionally distinct subsets: TH1 phenotype and TH2 phenotypes which differ in their cytokine and effector function.

Activated TH1 cells enhance cellular immunity (including an increase in antigen-specific CTL production) and are therefore of particular value in responding to intracellular infections. Activated TH1 cells may secrete one or more of IL-2, IFN-γ, and TNF-β. A TH1 immune response may result in local inflammatory reactions by activating macrophages, NK (natural killer) cells, and CD8 cytotoxic T cells (CTLs). A TH1 immune response may also act to expand the immune response by stimulating growth of B and T cells with IL-12. TH1 stimulated B cells may secrete IgG2a.

Activated TH2 cells enhance antibody production and are therefore of value in responding to extracellular infections. Activated TH2 cells may secrete one or more of IL-4, IL-5, IL-6, and IL-10.

A TH2 immune response may result production of IgG1, IgE, IgA memory B cells for future protection.

An enhanced immune response may include one or more of an enhanced TH1immune response and a TH2 immune response.

A TH1 immune response may include one or more of an increase in CTLs, an increase in one or more of the cytokines associated with a TH1 immune response (such as IL-2, IFN-65 , and TNF-β), an increase in activated macrophages, an increase in NK activity, or an increase in the production of IgG2a. Preferably, the enhanced TH1 immune response will include an increase in IgG2a production.

A TH1 immune response may be elicited using a TH1 adjuvant. A TH1 adjuvant will generally elicit increased levels of IgG2a production relative to immunization of the antigen without adjuvant. TH1 adjuvants suitable for use in the invention may include for example saponin formulations, virosomes and virus like particles, non-toxic derivatives of enterobacterial lipopolysaccharide (LPS), immunostimulatory oligonucleotides. Immunostimulatory oligonucleotides, such as oligonucleotides containing a CpG motif, are preferred TH1 adjuvants for use in the invention.

A TH2 immune response may include one or more of an increase in one or more of the cytokines associated with a TH2 immune response (such as IL-4, IL-5, IL-6 and IL-10), or an increase in the production of IgG1, IgE, IgA and memory B cells. Preferably, the enhanced TH2 immune response will include an increase in IgG1 production.

A TH2 immune response may be elicited using a TH2 adjuvant. A TH2 adjuvant will generally elicit increased levels of IgG1 production relative to immunization of the antigen without adjuvant. TH2 adjuvants suitable for use in the invention include, for example, mineral containing compositions, oil-emulsions, and ADP-ribosylating toxins and detoxified derivatives thereof. Mineral containing compositions, such as aluminium salts are preferred TH2 adjuvants for use in the invention.

Preferably, the invention includes a composition comprising a combination of a TH1 adjuvant and a TH2 adjuvant. Preferably, such a composition elicits an enhanced TH1 and an enhanced TH2 response, i.e., an increase in the production of both IgG1 and IgG2a production relative to immunization without an adjuvant. Still more preferably, the composition comprising a combination of a TH1 and a TH2 adjuvant elicits an increased TH1 and/or an increased TH2 immune response relative to immunization with a single adjuvant relative to immunization with a TH1 adjuvant alone or immunization with a TH2 adjuvant alone).

The immune response may be one or both of a TH1 immune response and a TH2 response. Preferably, immune response provides for one or both of au enhanced TH1 response and an enhanced TH2 response. Preferably, the immune response includes au increase in the production of IgG1 and/or IgG2 and/or IgGA.

The invention may be used to elicit systemic and/or mucosal immunity. The enhanced immune response may be one or both of a systemic and a mucosal immune response. Preferably, the immune response provides for one or both of an enhanced systemic and an enhanced mucosal immune response. Preferably the mucosal immune response is a TH2 immune response. Preferably, the mucosal immune response includes an increase in the production of IgA.

### Methods of Treatment, and Administration of the Vaccine

The invention also provides a method for raising an immune response in a mammal comprising the e step of administering an effective amount of a composition of the invention. The immune response is preferably protective and preferably involves antibodies and/or cell-mediated immunity. The method may raise a booster response.

The invention also provides a protein of the invention in combination with another antigen for combined use as a medicament e.g. for use in raising an immune response in a mammal.

The invention also provides the use of a protein of the invention in the manufacture of a medicament for raising an immune response in a mammal. By raising an immune response in the mammal by these uses and methods, the mammal can be protected against Chlamydia infection. More particularly, the mammal may be protected against Chlamydia trachomatis. The invention is effective against Chlamydia of various different serotypes, but can be particularly useful in protecting against disease resulting from Chlamydia infection by strains in serovar D.

Thus, according to a further aspect, the invention also provides a nucleic acid, protein, or antibody according to the invention for use as a medicament (e.g. a vaccine) or a diagnostic reagent. In one embodiment, the protein, nucleic acid or antibody is used for treatment, prevention or diagnosis of Chlamydia infection (preferably C. trachomatis) in a mammal. The invention also provides a method of treating, preventing of diagnosing Chlamydia infection (preferably, C. trachomatis infection) in a patient (preferably a mammal), comprising administering a therapeutically effective amount of a nucleic acid, protein or antibody of the invention.

Preferably, the nucleic acid, protein or antibody according to the invention is for treatment or prevention of Chlamydia infection or an associated condition (e.g. trachoma, blindness, cervicitis, pelvic inflammatory disease, infertility, ectopic pregnancy, chronic pelvic pain, salpingitis, urethritis, epididymitis, infant pneumonia, cervical squamous cell carcinoma, HIV infection, etc.), preferably, C. trachomatis infection. The immunogenic composition may additionally or alternatively be effective against C. pneumoniae.

The mammal is preferably a human. Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc. Thus a human patient may be less than 1 year old, 1-5 years old, 5-15 years old, 15-55 years old, or at least 55 years old. Preferred patients for receiving the vaccines are people going through purberty, teenagers, sexually active people, the elderly (e.g. ≧50 years old, ≧60 years old, and preferably ≧65 years), the young (e.g. ≦5 years old), hospitalised patients, healthcare workers, armed service and military personnel, pregnant women, the chronically ill, or immunodeficient patients. The vaccines are not suitable solely for these groups, however, and may be used more generally in a population.

Vaccines produced by the invention may be administered to patients at substantially the same time as (e.g. during the same medical consultation or visit to a healthcare professional or vaccination centre) other vaccines e.g. at substantially the same time as a human papillomavirus vaccine such as Cervarix® or Gardasil®; a tetanus, diphtheria and a cellular pertussis vaccine such as TDaP, DTaP or Boostrix®; a rubella vaccine such as MMR; or a tubercolosis vaccine such as the BCG. Examples of other vaccines that the vaccine produced by the invention may be administered at substantially the same time as are a measles vaccine, a mumps vaccine, a varicella vaccine, a MMRV vaccine, a diphtheria vaccine, a tetanus vaccine, a pertussis vaccine, a DTP vaccine, a conjugated H. influenzae type b vaccine, an inactivated poliovirus vaccine, a hepatitis B virus vaccine, a meningococcal conjugate vaccine (such as a tetravalent A-C-W135-Y vaccine), a respiratory syncytial virus vaccine, etc.

In a preferred embodiment, the protein of the invention is used to elicit antibodies that are capable of neutralising the proteolytic activity of Chlamydia HtrA, for example, of the wild-type Chlamydia HtrA. Neutralizing antibodies may be used as a vaccine capable of neutralising the activity of native HtrA expressed by infectious EB. In one embodiment, the protein of the invention is used to elicit antibodies that are capable of neutralising Chlamydia infectivity and/or virulence. Thus, the invention also provides the antibodies of the invention for neutralising wild-type Chlamydia HtrA proteins and/or Chlamydia infectivity and/or virulence.

The invention also provides the use of a nucleic acid, protein, or antibody of the invention in the manufacture of: (i) a medicament for treating or preventing bacterial infection; (ii) a diagnostic reagent for detecting the presence of bacteria or of antibodies raised against bacteria; and/or (iii) a reagent which can raise antibodies against bacteria. Said bacteria is preferably a Chlamydia, e.g. Chlamydia trachomatis or Chlamydia pneumoniae, but is preferably Chlamydia trachomatis.

Also provided is a method for diagnosing Chlamydia infection, comprising:

(a) raising an antibody against a protein of the invention;

(b) contacting the antibody of step (a) with a biological sample suspected of being infected with Chlamydia under conditions suitable for the formation of antibody-antigen complexes; and

(c) detecting said complexes, wherein detection of said complex is indicative of Chlamydia infection.

Proteins of the invention can be used in immunoassays to detect antibody levels (or, conversely, antibodies of the invention can be used to detect protein levels). Immunoassays based on well defined, recombinant antigens can be developed to replace invasive diagnostics methods. Antibodies to proteins within biological samples, including for example, blood or serum samples, can be detected. Design of the immunoassays is subject to a great deal of variation, and a variety of these are known in the art. Protocols for the immunoassay may be based, for example, upon competition, or direct reaction, or sandwich type assays. Protocols may also, for example, use solid supports, or may be by immunoprecipitation. Most assays involve the use of labeled antibody or polypeptide; the labels may be, for example, fluorescent, chemiluminescent, radioactive, or dye molecules. Assays which amplify the signals from the probe are also known; examples of which are assays which utilize biotin and avidin, and enzyme-labeled and mediated immunoassays, such as ELISA assays.

Kits suitable for immunodiagnosis and containing the appropriate labeled reagents are constructed by packaging the appropriate materials, including the compositions of the invention, in suitable containers, along with the remaining reagents and materials (for example, suitable buffers, salt solutions, etc.) required for the conduct of the assay, as well as suitable set of assay instructions.

### Testing Efficacy of Compositions

The efficacy of the immunogenic compositions of the present invention can be evaluated in in vitro and in vivo animal models prior to host, e.g., human, administration. For example, in vitro neutralization by Peterson et al (1988) is suitable for testing vaccine compositions directed toward Chlamydia trachomatis.

One way of checking efficacy of therapeutic treatment involves monitoring C. trachomatis infection after administration of the compositions of the invention. One way of checking efficacy of prophylactic treatment involves monitoring immune responses both systemically (such as monitoring the level of IgG1 and IgG2a production) and mucosally (such as monitoring the level of IgA production) against the Chlamydia trachomatis antigens in the compositions of the invention after administration of the composition. Typically, serum Chlamydia specific antibody responses are determined post-immunisation but pre-challenge whereas mucosal Chlamydia specific antibody body responses are determined post-immunisation and post-challenge.

One example of such an in vitro test is described as follows. Hyper-immune antisera is diluted in PBS containing 5% guinea pig serum, as a complement source. Chlamydia trachomatis (10⁴ IFU; inclusion forming units) are added to the antisera dilutions. The antigen-antibody mixtures are incubated at 37° C. for 45 minutes and inoculated into duplicate confluent Hep-2 or HeLa cell monolayers contained in glass vials (e.g., 15 by 45 mm), which have been washed twice with PBS prior to inoculation. The monolayer cells are infected by centrifugation at 1000× g for 1 hour followed by stationary incubation at 37° C. for 1 hour. Infected monolayers are incubated for 4 or hours, fixed and stained with Chlamydia specific antibody, such as anti-MOMP. Inclusion-bearing cells are counted in ten fields at a magnification of 200×. Neutralization titer is assigned on the dilution that gives 50% inhibition as compared to control monolayers/IFU.

Another way of assessing the immunogenicity of the compositions of the present invention is to express the proteins recombinantly for screening patient sera Of mucosal secretions by immunoblot and/or microarrays. A positive reaction between the protein and the patient sample indicates that the patient has mounted an immune response to the protein in question. This method may also be used to identify immunodominant antigens and/or epitopes within antigens.

The efficacy of vaccine compositions can also be determined in vivo by challenging animal models of Chlamydia trachomatis infection, e.g., guinea pigs or mice, with the vaccine compositions. For example, in vivo vaccine composition challenge studies in the guinea pig model of Chlamydia trachomatis infection can be performed. A description of one example of this type of approach follows. Female guinea pigs weighing 450-500 g are housed in an environmentally controlled room with a 12 hour light-dark cycle and immunized with vaccine compositions via a variety of immunization routes. Post-vaccination, guinea pigs are infected in the genital tract with the agent of guinea pig inclusion conjunctivitis (GPIC), which has been grown in HeLa or McCoy cells (Rank et al. (1988)). Each animal receives approximately 1.4×1.0⁷ inclusion forming units (IFU) contained in 0.05 ml of sucrose-phosphate-glutamate buffer, pH 7.4 (Schacter, 1980). The course of infection monitored by determining the percentage of inclusion-bearing cells by indirect immunofluorescence with GPIC specific antisera, or by Giemsa-stained smear from a scraping from the genital tract (Rank et al 1988). Antibody titers in the serum is determined by an enzyme-linked immunosorbent assay.

Alternatively, in vivo vaccine compositions challenge studies can be performed in the murine model of Chlamydia trachomatis (Morrison et al 1995). A description of one example of this type of approach is as follows. Female mice 7 to 12 weeks of age receive 2.5 mg of depo-provera subcutaneously at 10 and 3 days before vaginal infection. Post-vaccination, mice are infected in the genital tract with 1,500 inclusion-forming units of Chlamydia trachomatis contained in 5 ml of sucrose-phosphate-glutamate buffer, pH 7.4. The course of infection is monitored by determining the percentage of inclusion-bearing cells by indirect immunofluorescence with Chlamydia trachomatis specific antisera, or by a Giemsa-stained smear from a scraping from the genital tract of an infected mouse. The presence of antibody titers in the serum of a mouse is determined by an enzyme-linked immunosorbent assay.

### Nucleic Acid Immunisation

The immunogenic compositions described above include Chlamydia antigens. in all cases, however, the polypeptide antigens can be replaced by nucleic acids (typically DNA) encoding those polypeptides, to give compositions, methods and uses based on nucleic acid immunisation. Nucleic acid immunisation is now a developed field (e.g. see Donnelly et al. (1997) Anna Rev Immunol 15:617-648; Strugnell el al. (1997) Immunol Cell Biol 75(4):364-369; Cui (2005), Adv Genet 54:257-89; Robinson & Torres (1997) Seminars in Immunol 9:271-283; Brunham et al. (2000) J Infect Dis 181 Suppl 3:5538-43; Svanholm et al. (2000) Scand J Immunol 51(4):345-53; DNA Vaccination—Genetic Vaccination (1998) eds. Koprowski et al. (ISBN 3540633928); Gene Vaccination: Theory and Practice (1998) ed. Raz (ISBN 3540644288), etc.).

The nucleic acid encoding the immunogen is expressed in vivo after delivery to a patient and the expressed immunogen then stimulates the immune system. The active ingredient will typically take the form of a nucleic acid vector comprising: (i) a promoter; (ii) a sequence encoding the immunogen, operably linked to the promoter; and optionally (iii) a selectable marker. Preferred vectors may further comprise (iv) an origin of replication; and (v) a transcription terminator downstream of and operably linked to (ii). In general, (i) & (v) will be eukaryotic and (iii) & (iv) will be prokaryotic.

Preferred promoters are viral promoters e.g. from cytomegalovirus (CMV). The vector may also include transcriptional regulatory sequences (e.g. enhancers) in addition to the promoter and which interact functionally with the promoter. Preferred vectors include the immediate-early CMV enhancer/promoter, and more preferred vectors also include CMV intron A. The promoter is operably linked to a downstream sequence encoding an immunogen, such that expression of the immunogen-encoding sequence is under the promoter's control.

Where a marker is used, it preferably functions in a microbial host (e.g. in a prokaryote, in a bacteria, in a yeast). The marker is preferably a prokaryotic selectable marker (e.g. transcribed under the control of a prokaryotic promoter). For convenience, typical markers are antibiotic resistance genes.

The vector of the invention is preferably an autonomously replicating episomal or extrachromosomal vector, such as a plasmid.

The vector of the invention preferably comprises an origin of replication. It is preferred that the origin of replication is active in prokaryotes but not in eukaryotes.

Preferred vectors thus include a prokaryotic marker for selection of the vector, a prokaryotic origin of replication, but a eukaryotic promoter for driving transcription of the immunogen-encoding sequence. The vectors will therefore (a) be amplified and selected in prokaryotic hosts without polypeptide expression, but (b) be expressed in eukaryotic hosts without being amplified. This arrangement is ideal for nucleic acid immunization vectors.

The vector of the invention may comprise a eukaryotic transcriptional terminator sequence downstream of the coding sequence. This can enhance transcription levels. Where the coding sequence does not have its own, the vector of the invention preferably comprises a polyadenylation sequence. A preferred polyadenylation sequence is from bovine growth hormone.

The vector of the invention may comprise a multiple cloning site.

In addition to sequences encoding the immunogen and a marker, the vector may comprise a second eukaryotic coding sequence. The vector may also comprise an IRES upstream of said second sequence in order to permit translation of a second eukaryotic polypeptide from the same transcript as the immunogen. Alternatively, the immunogen-coding sequence may be downstream of an IRES.

The vector of the invention may comprise unmethylated CpG motifs e.g. unmethylated DNA sequences which have in common a cytosine preceding a guanosine, flanked by two 5′ purines and two 3′ pyrimidines. In their unmethylated form these DNA motifs have been demonstrated to be potent stimulators of several types of immune cell.

Vectors may be delivered in a targeted way. Receptor-mediated DNA delivery techniques are described in, for example, Findeis et al., Trends Biotechnol (1993) 11:202; Chiou et al. (1994) Gene Therapeutics: Methods And Applications Of Direct Gene Transfer ed. Wolff; Wu et al., J. Biol. Chem. (1988) 263:621; Wu et al., J. Biol. Chem. (1994) 269:542; Zenke et al., Proc. Natl. Acad. Sci. (USA) (1990) 87:3655; and Wu et al., J. Biol. Chem. (1991) 266:338.

Therapeutic compositions containing a nucleic acid are administered in a range of about 100 ng to about 200 mg of DNA for local administration in a gene therapy protocol. Concentration ranges of about 500 ng to about 50 mg, about lug to about 2 mg, about 5 μg to about 500 μg, and about 20 μg to about 100 μg of DNA can also be used during a gene therapy protocol. Factors such as method of action (e.g. for enhancing or inhibiting levels of the encoded gene product) and efficacy of transformation and expression are considerations which will affect the dosage required for ultimate efficacy. Where greater expression is desired over a larger area of tissue, larger amounts of vector or the same amounts re-administered in a successive protocol of administrations, or several administrations to different adjacent or close tissue portions may be required to effect a positive therapeutic outcome. In all cases, routine experimentation in clinical trials will determine specific ranges for optimal therapeutic effect.

Vectors can be delivered using gene delivery vehicles. The gene delivery vehicle can be of viral or non-viral origin (see generally Jolly, Cancer Gene Therapy (1994) 1:51; Kitmura, Human Gene Therapy (1994) 5:845; Connelly, Human Gene Therapy (1995) 1:185; and Kaplitt, Nature Genetics (1994) 6:148).

Viral-based vectors for delivery of a desired nucleic acid and expression in a desired cell are well known in the art. Exemplary viral-based vehicles include, but are not limited to, recombinant retroviruses (e.g. WO 90/07936; WO 94/03622; WO 93/25698; WO 93/25234; U.S. Pat. No. 5,219,740; WO 93/11230; WO 93/10218; U.S. Pat. No. 4,777,127; GB Patent No. 2,200,651; EP-A-0345242; and WO 91/02805), alphavirus-based vectors (e.g. Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532); hybrids or chimeras of these viruses may also be used), poxvirus vectors (e.g. vaccinia, fowlpox, canarypox, modified vaccinia Ankara, etc.), adenovirus vectors, and adeno-associated virus (AAV) vectors (e.g. see WO 90/07936; WO 94/03622; WO 93/25698; WO 93/25234; U.S. Pat. No. 5,219,740; WO 93/11230; WO 93/10218; U.S. Pat. No. 4,777,127; GB Patent No. 2,200,651; EP-A-0345242; WO 91/02805; WO 94/12649; WO 93/03769; WO 93/19191; WO 94/28938; WO 95/11984; and WO 95/00655). Administration of DNA linked to killed adenovirus (Curiel, Hum. Gene Ther. (1992) 3:147) can also be employed.

Non-viral delivery vehicles and methods can also be employed, including, but not limited to, polycationic condensed DNA linked or unlinked to killed adenovirus alone (e.g. De Libero et al, Nature Reviews Immunology, 2005, 5: 485-496), ligand-linked DNA (Wu, J. Biol. Chem. (1989) 264:16985), eukaryotic cell delivery vehicles cells (U.S. Pat. No. 5,814,482; WO 95/07994; WO 96/17072; WO 95/30763; and WO 97/42338) and nucleic charge neutralization or fusion with cell membranes. Naked DNA can also be employed. Exemplary naked DNA introduction methods are described in WO 90/11092 and U.S. Pat. No. 5,580,859. Liposomes (e.g. immunoliposomes) that can act as gene delivery vehicles are described in U.S. Pat. No. 5,422,120; WO 95/13796; WO 94/23697; WO 91/14445; and EP-0524968. Additional approaches are described in Philip, Mol. Cell Biol. (1994)/4;2411 and Woffendin, Proc. Natl. Acad. Sci. (1994) 91:11581.

Further non-viral delivery suitable for use includes mechanical delivery systems such as the approach described in Donnelly et at. (1997) Annu Rev Immunol 15:617-648. Moreover, the coding sequence and the product of expression of such can be delivered through deposition of photopolymerized hydrogel materials or use of ionizing radiation (e.g. U.S. Pat. No. 5,206,152 and WO 92/11033). Other conventional methods for gene delivery that can be used for delivery of the coding sequence include, for example, use of hand-held gene transfer particle gun (U.S. Pat. No. 5,149,655) or use of ionizing radiation for activating transferred genes (Strugnell et al. (1997) Immunol Cell Biol 75(4):364-369 and Cui (2005) Adv Genet 54:257-89).

Delivery DNA using PLG {poly(lactide-co-glycolide)} microparticles is a particularly preferred method e.g. by adsorption to the microparticles, which are optionally treated to have a negatively-charged surface (e.g. treated with SDS) or a positively-charged surface (e.g. treated with a cationic detergent, such as CTAB).

### Antibody Immunisation

The antibodies of the invention may be used, for example, for neutralising the proteolytic activity of the wild-type HtrA protein. Antibodies against Chlamydia antigens can be used for passive immunisation (Brandt et al. (2006) J Antimicrob Chemother. 58(6):1291-4. Epub 2006 Oct. 26). Thus the invention provides the use of antibodies of the invention in therapy. The invention also provides the use of such antibodies in the manufacture of a medicament. The invention also provides a method for treating a mammal comprising the step of administering an effective amount of an antibody of the invention. As described above for immunogenic compositions, these methods and uses allow a mammal to be protected against Chlamydia infection.

### Processes

According to further aspects, the invention provides various processes.

The invention also provides a process for reducing or eliminating the protease activity of a wild-type Chlamydia HtrA protein, comprising mutating one or more amino acid residues of the protein, wherein the resulting protein retains the ability to elicit an immune response, such as a cell-mediated and/or an antibody response, against the wild-type Chlamydia HtrA protein. This may conveniently be achieved by performing, site-directed mutagenesis on a nucleic acid encoding the HtrA protein. Preferred mutations are discussed above. In one embodiment, the mutation is not S247A in C. trachomatis serovar L2. In another embodiment, the mutation is not S247A. In a further embodiment, the mutation is not of the serine in the catalytic triad. The invention further provides a Chlamydia HtrA protein obtainable by this process.

A process for producing a protein of the invention is provided, comprising the step of culturing a host cell of the invention under conditions which induce protein expression.

A process for producing protein or nucleic acid of the invention is provided, wherein the protein or nucleic acid is synthesised in part or in whole using chemical means.

A process for detecting Chlamydia (preferably C. trachomatis) in a biological sample is also provided, comprising the step of contacting a nucleic acid according to the invention with the biological sample under hybridising conditions. The process may involve nucleic acid amplification (e.g. PCR, SDA, SSSR, LCR, TMA etc.) or hybridisation (e.g. microarrays, blots, hybridisation with probe in solution etc.),

A process for detecting wild-type Chlamydia HtrA (preferably, C. trachomatis HtrA) is provided, comprising the steps of: (a) contacting an antibody of the invention with a biological sample under conditions suitable for the formation of an antibody-antigen complexes; and (b) detecting said complexes. This process may advantageously be used to diagnose Chlamydia infection.

### General

The practice of the present invention will employ, unless otherwise indicated, conventional methods of chemistry, biochemistry, molecular biology, immunology and pharmacology, within the skill of the art. Such techniques are explained fully in the literature. See, e.g., Gennaro (2000) Remington: The Science and Practice of Pharmacy. 20th edition, ISBN: 0683306472; Methods In Enzymology (S. Colowick and N. Kaplan, eds., Academic Press, Inc.); Handbook of Experimental Immunology, Vols. I-IV (D. M. Weir and C. C. Blackwell, eds, 1986, Blackwell Scientific Publications); Sambrook et al. (2001) Molecular Cloning: A Laboratory Manual, 3rd edition (Cold Spring Harbor Laboratory Press); Handbook of Surface and Colloidal Chemistry (Birdi, K. S. ed., CRC Press, 1997); Ausubel et al. (eds) (2002) Short protocols in molecular biology, 5th edition (Current Protocols); Molecular Biology Techniques: An intensive Laboratory Course, (Ream et al., eds., 1998, Academic Press); and PCR (Introduction to Biotechniques Series), 2nd ed. (Newton & Graham eds., 1997, Springer Verlag) etc.

“G1” numbering is used herein. A G1 number, or “GenInfo Identifier”, is a Series of digits assigned consecutively to each sequence record processed by NCBI when sequences are added to its databases. The GI number bears no resemblance to the accession number of the sequence record. When a sequence is updated (e.g. for correction, or to add more annotation or information) then it receives a new G1 number. Thus the sequence associated with a given GI number is never changed. Where the invention concerns an “epitope”, this epitope may be a B-cell epitope and/or a T-cell epitope. Such epitopes can be identified empirically (e.g. using PEPSCAN (Geysen et al. (1984) PNAS USA 81:3998-4002; Carter (1994) Methods Mol Biol 36:207-23) or similar methods), or they can be predicted (e.g. using the Jameson-Wolf antigenic index (Jameson, B A et al. 1988, CABIOS 4(1):181-186), matrix-based approaches (Raddrizzani & Hammer (2000) Brief Bioinform 1(2):179-89). MAPITOPE (Bublil et al. (2007) Proteins 68(1):294-304), TEPITOPE (De Lalla et al. (1999) J. Immunol. 163:1725-29; Kwok et al. (2001) Trends Immunol 22:583-88), neural networks (Brusic et al. (1998) Bioinformatics 14(2):121-30), OptiMer & EpiMer (Meister et al. (1995) Vaccine 13(6):581-91; Roberts et al. (1996) AIDS Res Hum Retroviruses 12(7):593-610), ADEPT (Maksyutov & Zagrebelnaya (1993) Comput Appl Biosci 9(3):291-7), Tsites (Feller & de la Cruz (1991) Nature 349(6311):720-1), hydrophilicity (Hopp (1993) Peptide Research 6:183-190), antigenic index (Welling et al. (1985) FEBS Lett. 188:215-218) or the methods disclosed in Davenport et al. (1995) Immunogenetics 42:392-297; Tsurui & Takahashi (2007) J Pharmacol Sci. 105(4):299-316; Tong at al. (2007) Brief Bioinform. 8(2):96-108 Schirle et al. (2001) J Immunol Methods. 257(1-2):1-16; and Chen et al. (2007) Amino Acids 33(3):423-8, etc.). Epitopes are the parts of an antigen that are recognised by and bind to the antigen binding sites of antibodies or T-cell receptors, and they may also be referred to as “antigenic determinants”.

Where an antigen “domain” is omitted, this may involve omission of a signal peptide, of a cytoplasmic domain, of a transmembrane domain, of an extracellular domain, etc.

The term “comprising” encompasses “including” as well as “consisting” e.g. a composition “comprising” X may consist exclusively of X or may include something additional e.g. X+Y.

The term “about” in relation to a numerical value x means, for example, x±10%.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of Current Protocols in Molecular Biology (F. M. Ausubel et al., eds., 1987) Supplement 30. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in Smith & Waterman (1981) Adv. Appl. Math. 2: 482-489.

### BRIEF DESCRIPTION OF DRAWINGS

FIG. 1 shows an SDS-PAGE and Coomassie staining showing the results obtained with the C. trachomatis HtrAs. From left to right, the lanes are as follows: 1: Molecular weight markers. 2: CT823-(H142R). 3: CT823-(H142R+DTT). 4: CT823-(H142R+BSA). 5: CT823-(H142R+BSA+DTT). 6: BSA. 7: BSA+DTT. 8: CT823-(H143R). 9: CT823-(H143R+DTT). 10: CT823-(H143R+BSA). 11: CT823-(H143R -+BSA+DTT). 12: CT823-(S247A). 13: CT823-(S247A+DTT). 14: CT823-(S247A+BSA). 15: CT823-(S247A+BSA+OTT). 16: wild-type 823+DTT. 17: wild-type 823+BSA. 18: CT823-(wild-type 823+BSA+DTT). Section A of the gel represents the area where the intact BSA bands migrate; Section B of the gel represents the area where the HtrA bands migrates; Section C of the gel represents the area where the main portion of the degradation products of BSA migrate.

FIG. 2 shows an SDS-PAGE and Coomassie staining showing the results obtained with the C. muridarum HtrAs. From left to right the lanes are as follows: 1: Molecular weight markers; 2: TC0210-(H143R)+DTT; 3: TC0210-(H143R)+BSA-+DTT; 4: Wild type TC0210+DTT; 5: Wild type TC0210+BSA+DTT.

FIG. 3 shows an SDS-PAGE and Coomassie staining showing the results obtained digesting the Cm MOMP with wild type and with mutant TC0210. From left to right the lanes are as follows: 1: Molecular Weight Markers. 2: wild-type TC0210+DTT. 3: TC0210-(H143R)−DTT. 4: MOMP+DTT. 5: wild-type TC0210+MOMP, 6: TC0210-(H443R)+MOMP. 7: wild-type TC0210+MOMP+DTT. 8: TC0210-(H143R)+MOMP+DTT.

FIG. 4 shows the frequency of HtrA specific CD4-Th1 cells in mice immunized with the TC0210 wild-type or TC0210-(H143R) mutant in response to specific stimuli (from left to right: EB, TC210, TC210R (H143R mutant), CT823, CT823R (H143R mutant) and TC660). The Y axis shows the number of CD4 T cells (frequency on 10⁵ CD4) producing IFN; and IL2/TNF in PBMC of mice immunized with the antigen. Each of the six groups of four bars shows data for (from left to right): live EB, LTK63+CpG; TC0210+adjuvant; or TC0210-H143R+adjuvant.

FIG. 5 shows the protective activity of TC0210wild-type and TC0210 (H143R) mutant. The graph shows log10 IFU/lung for gour groups (left to right: 10³ live EBs; LTK63+CpG; TC0210; H243R)

FIG. 6 shows the amino acid (SEQ ID NO: 1) and nucleic acid (SEQ ID NO: 2) sequences of the CT823 HtrA protein from C. trachomatis. The residues of the catalytic triad are underlined.

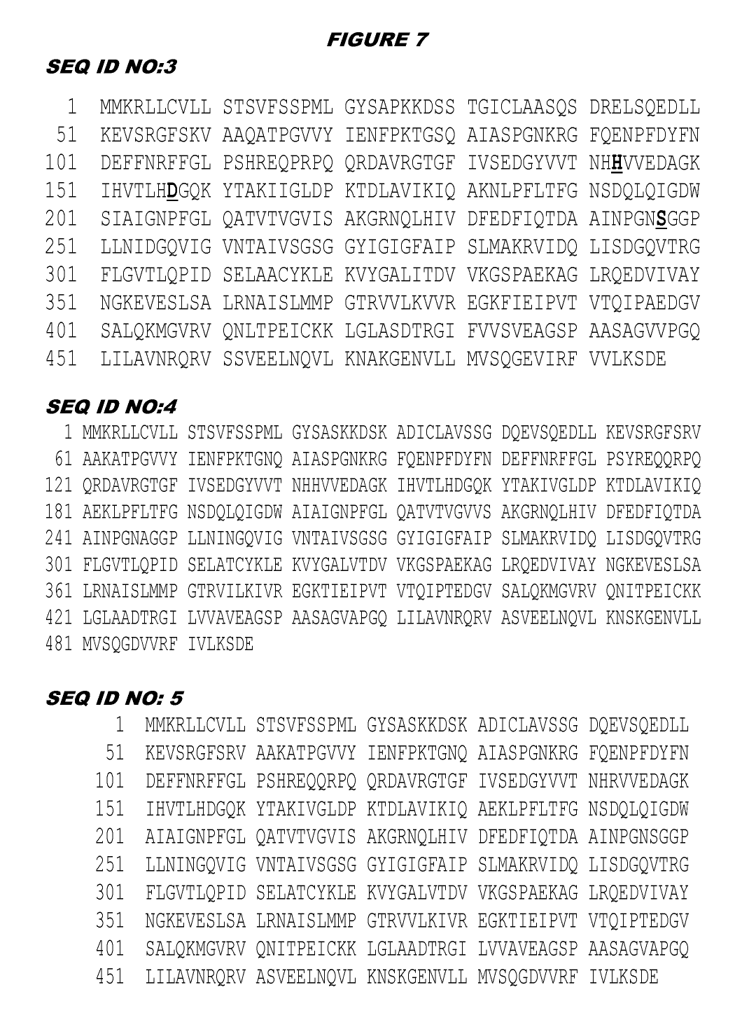

FIG. 7 shows the amino acid sequences of the TC0210 HtrA protein from C. muridarum, the C. trachomatis serovar L2 HtrA with the S247A mutation, and C. trachomatis CT823 with the H143R mutation.

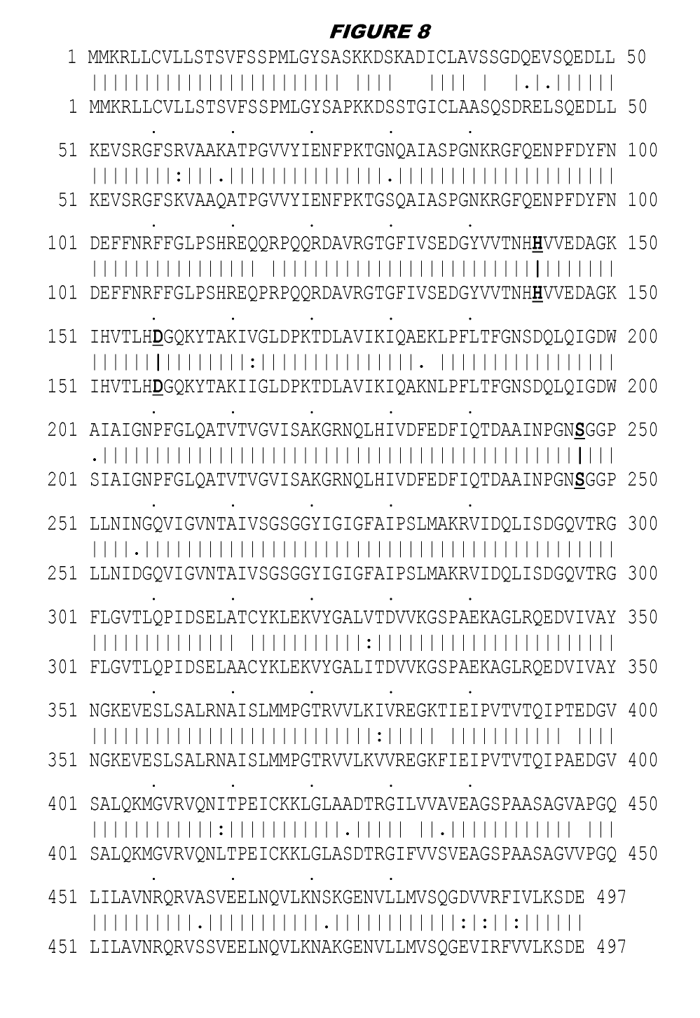

FIG. 8 shows a sequence alignment of the CT823 (SEQ ID NO: 1) and TC0210 (SEQ ID NO: 3) proteins. The residues of the catalytic triad are underlined.

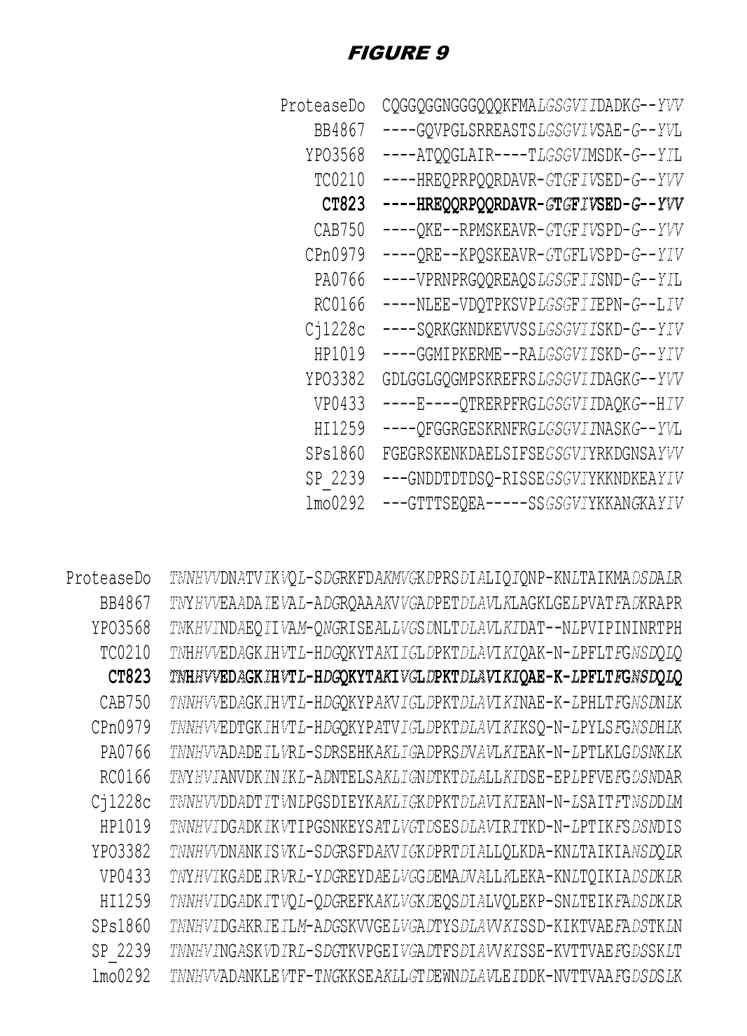

FIG. 9 shows a sequence alignment of the DegP protease domain of E. coli (ProteaseD₀; ID b0161 from aa 95 to aa 277) with the protease regions of Bordetella Bronchiseptica DegQ (BB4867), Chlamydia muridarum HtrA (TC0210), Chlamydia trachomatis serovar D HtrA (CT823), Chlamydophila abortus HtrA (CAB750), Chlamydophila pneumoniae CWL029 HtrA (CPn0979), Pseudomonas aeruginosa PAO1 (PA0766), Rickettsia conorii (RC0166), Campylobacter jejuni NCTC11168 (Cj1228c), Helicobacter pylori 26695 (HP1019), Yersinia pestis DO92 DegP (YPO3382), Vibrio parahaemolyticus HtrA (VP0433), Yersinia pestis CO92 DegS (YPO3568), Streptococcus pyogenes SSI-1 serotype M3 HtrA (SPs1860), Streptococcus pneumoniae TIGR4 (SP_{—}2239), Haemophilus influenzae Serotype D HtrA (H11259), and Listeria monocytogenes EGD-e (Imo0292). The Alignment was constructed using the MultAlin program (Multiple sequence alignment with hierarchical clustering. F. Corpet, Nucl. Acids Res., 16 (22), 10881-10890 (1988)). Similar results can be obtained using the Clustalw from GCG Wisconsin Package.

FIG. 10 shows a sequence alignment of the serovar L2 HtrA protein of SEQ ID NO:4, which has an S247A mutation (subject sequence, bottom line) and the wild-type CT823 protein of SEQ ID NO:1 (query sequence, top line).

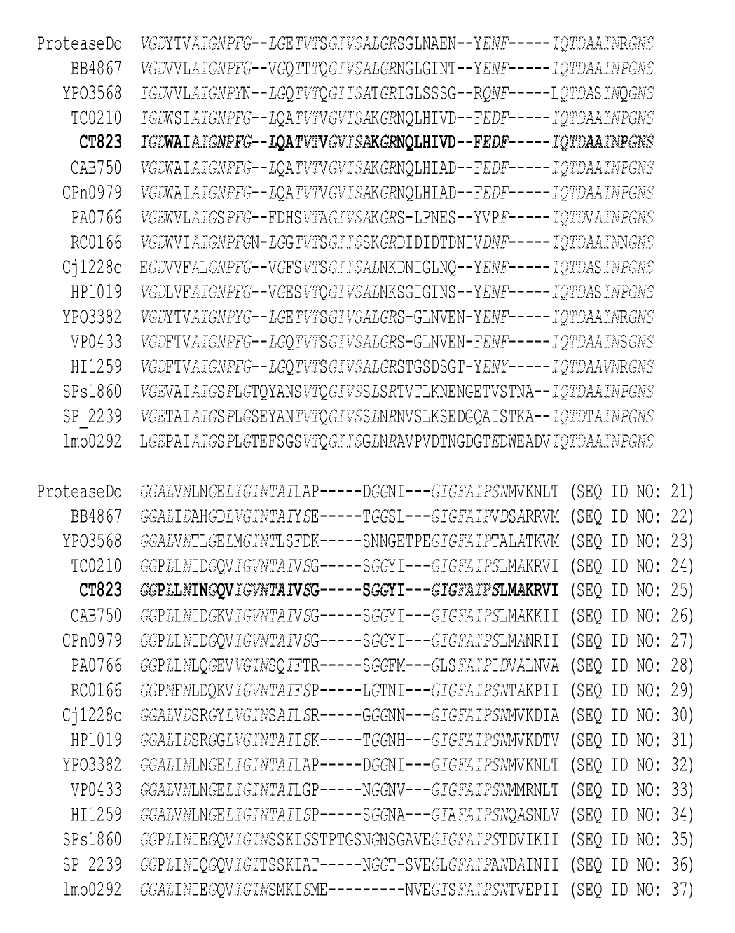

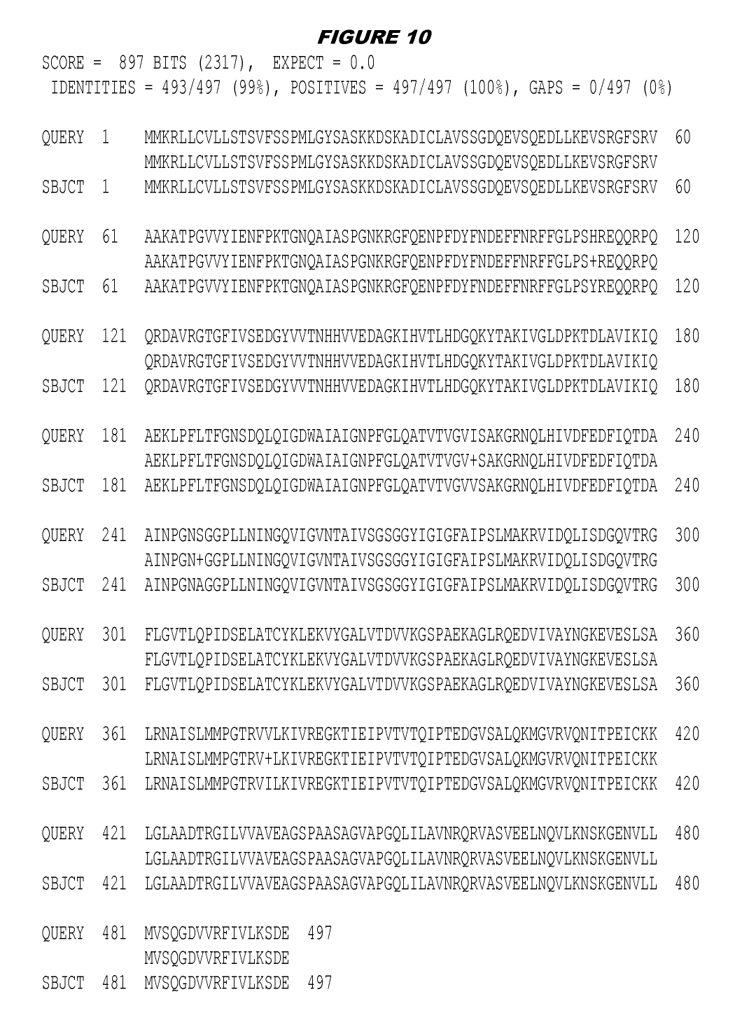

### BRIEF DESCRIPTION OF SEQUENCE LISTING

| SEQ ID NO: | Description |
|---|---|
| 1/2 | C. trachomatis HtrA |
| 3 | C. muridarum HtrA |
| 4 | Huston et al. HtrA mutant |
| 5 | HtrA mutant |
| 6 | CT372/hypothetical protein (AAC67968) |
| 7 | CT443/omcB (AAC68042) |
| 8 | CT043/hypothetical protein (AAC67634) |
| 9 | CT153/hypothetical protein (AAC67744) |
| 10 | CT279/nqr3 (AAC67872) |
| 11 | CT601/papQ (AAC68203) |
| 12 | CT711/hypothetical protein (AAC68306) |
| 13 | CT114/hypothetical protein (AAC67705) |
| 14 | CT480/oppA_4 (AAC68080) |
| 15 | CT456/hypothetical protein (AAC68056) |
| 16 | CT381/ArtJ (AAC67977) |
| 17 | CT089/lcrE (AAC67680) |
| 18 | CT734/hypothetical protein (AAC68329) |
| 19 | CT016/hypothetical protein (AAC67606) |
| 20 | CT733/hypothetical protein (AAC68328) |
| 21-37 | Sequences aligned in FIG. 9 |

### MODES FOR CARRYING OUT THE INVENTION

### EXAMPLE 1

To avoid potential degradation by chlamydial HtrA of other antigens that eventually compose vaccine doses, some mutants of both the C. trachomatis HtrA (CT823) and the C. muridarum HtrA (TC0210) were created, and their protease activity was compared to the protease activity of the recombinant wild type antigen versions.

To try to reduce or eliminate the protease activity of CT823 and TC0210, a series of mutants in the catalytic triad of the protease domain was created: CT823-(H142R), CT823-(H143R), CT823-(S247A), and TC0210-(H143R).

The recombinant wild type and mutant proteins were cloned and expressed in E. coli and were then purified and used to see if the mutations were able to reduce or to eliminate the protease activity.

Protease activity of HtrAs was studied by performing a digestion consisting of the following steps:

mixing wild type or a mutant HtrA protein with BSA (substrate) in the presence or absence of the reducing agent DTT;

incubating the mixture overnight at 37° C.;

separating the resulting proteins by means of polyacrylamide gel electrophoresis SDS-Page);

staining the gels with Coomassie R-250 Brilliant Blue; and

evaluating the results

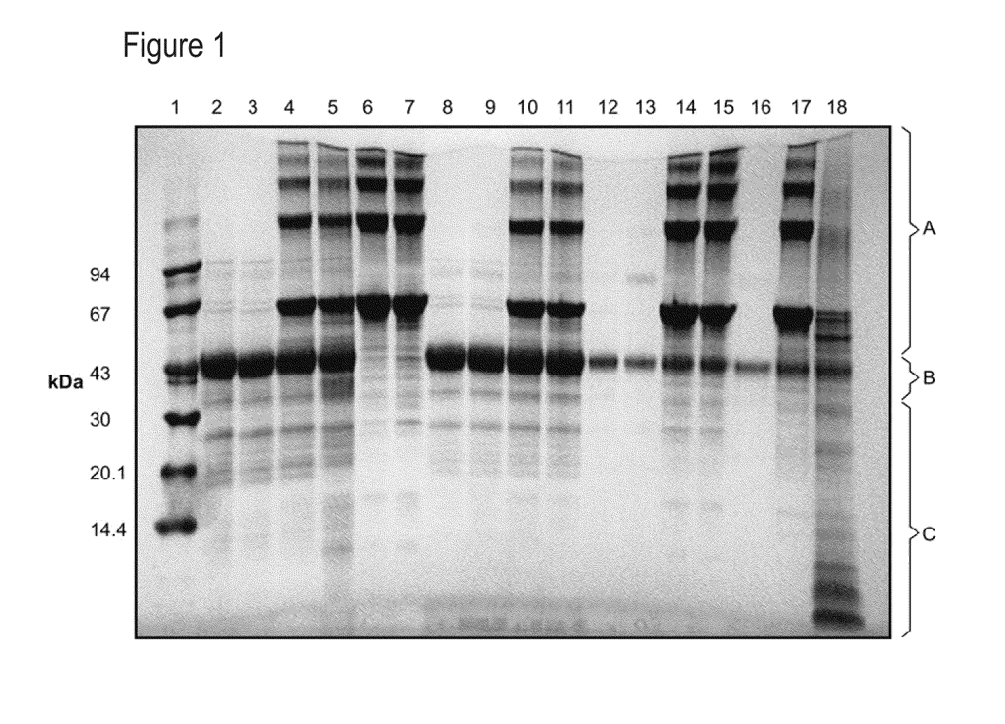

The results are shown in FIG. 1. The wild-type CT823 (lane 16) was shown to possess a strong protease activity in the presence of the reducing agent DTT (dithiothreitol) as shown by degradation of the intact BSA protein (lane 7) when incubated with wild type CT823 protein in presence of DTT (lane 18) but not in the absence of DTT (lane 17).

The mutant CT823-(H142R) (lanes 2 and 3) was found not to degrade BSA in the absence of the reducing agent DTT (lane 4) and was found to degrade a low amount of BSA in the presence of DTT (lane 5). This is suggested by the presence of some additional low molecular weight bands that are visible when the mutant is incubated with BSA in the presence of DTT (lane 5) but not in the absence of DTT (lane 4).

The mutant CT823-(H143R) (lanes 8 and 9) was found not to degrade BSA, either in the absence of DTT (lane 10) or in the presence of DTT (lane 11).

Further, the mutant CT823-(S247A) was found to completely lose its protease activity. Thus, the CT823-(S247A) mutant behaves similarly to the CT823-(H143R) mutant.

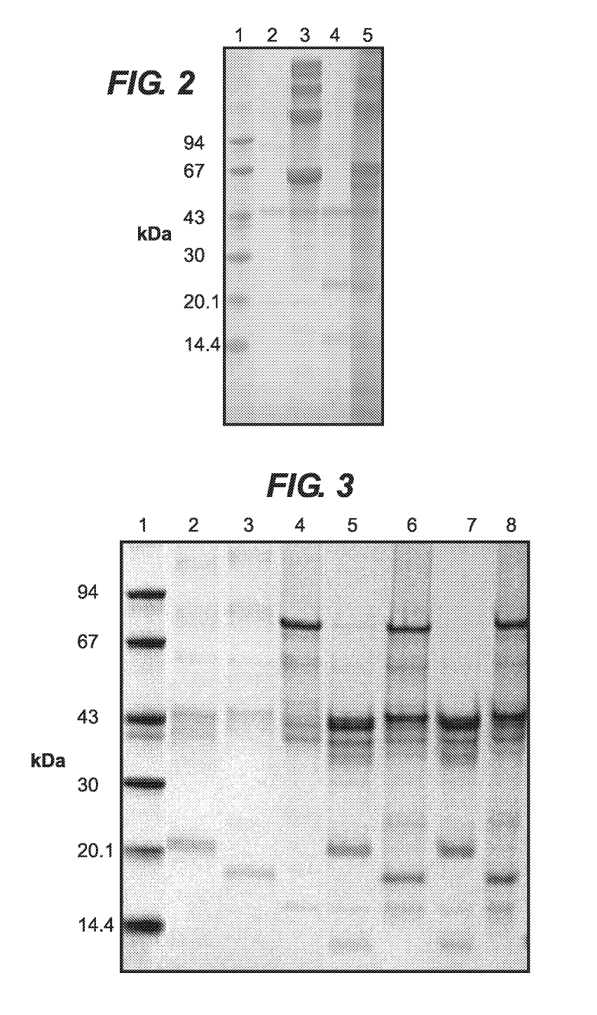

FIG. 2 shows that the wild type cloned version of TC0210 (lane 4) possesses a strong protease activity as it degrades the BSA substrate (lane 5). In contrast, the mutant T0210-(H143R) (lane 2) does not degrade the BSA substrate (lane 3).

The results obtained in the experimental conditions used suggest that the recombinant HtrA from C. trachomatis (CT823) and C. muridarum (TC0210) possess a marked protease activity. The CT823-(H142R) mutant was found to exhibit a very strong reduction of the protease activity. The protease activity of the CT823-(H143R), CT823-(S247A) and TC0210-(H143R) mutants was eliminated.

### EXAMPLE 2

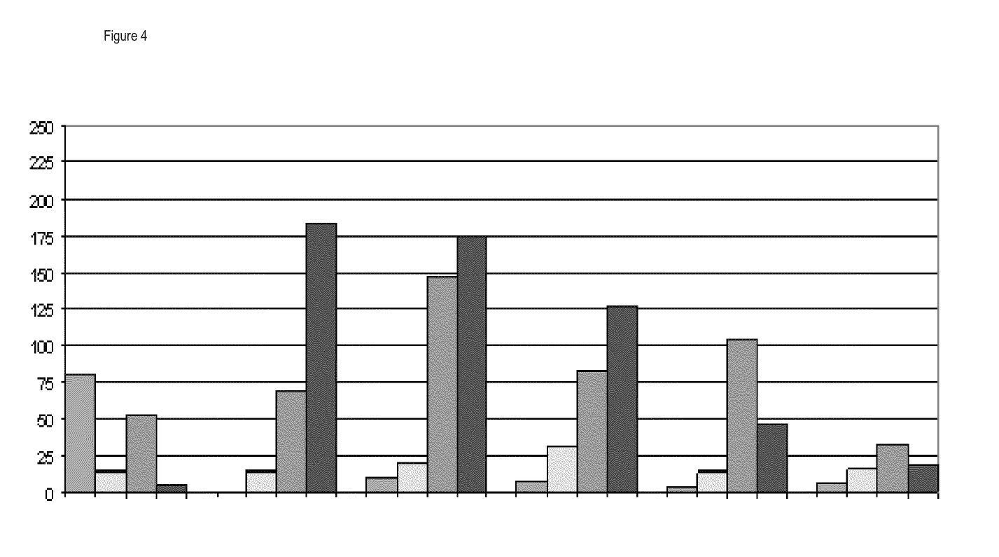

The C. muridarum Major Outer Membrane Protein (MOMP) was used as the substrate in a digestion assay with wild type and mutant C. muridarum HtrA (TC0210) in the presence or in the absence of the reducing agent Dithiothreitol (DTT). FIG. 3 shows that the wild type TC0210 (lane 2) completely degrades MOMP protein in the presence of DTT (lane 7) and strongly degrades MOMP protein in the absence of DTT (lane 5). In contrast, the mutant TC0210-(H143R) does not degrade the MOMP protein, either in the presence of DTT (lane 8) or in the absence of DTT (lane 6).

### EXAMPLE 3

The ability of the TC0210-(h143R) mutant protein to stimulate CD4+ IFN+−γ cells in PBMC purified from mice immunized with the antigen was evaluated in the Chlamydia muridarum mouse infection model. The animal model common used for C. trachomatis infections consists in three immunizations with C. muridarum antigens formulations for each mouse and an intranasal challenge with C. muridarum live EBs. C. muridarum is the species which naturally infects the mice and causes persistent diseases.

Groups of mice were immunized with either TC0210-HIS(wild-type) or TC0210-H143R recombinant antigens formulated with the LTK63+CpG adjuvant (3 doses of 15 ug protein, at 2 week intervals, given intramuscularly). As a negative control, mice were immunized with the adjuvant only. A group of mice that received a primary and a secondary C. muridarum infection were also included as a protection control. Two weeks after the last immunization, PBMC were purified from blood samples of immunized mice and tested for the presence of antigen-specific CD4-Th1 cells using in vitro stimulation assays followed by multiparametric staining of IFN+-CD4+ T cells using both wild-type and mutant HtrA, from C. trachomatis and C. muridarum.

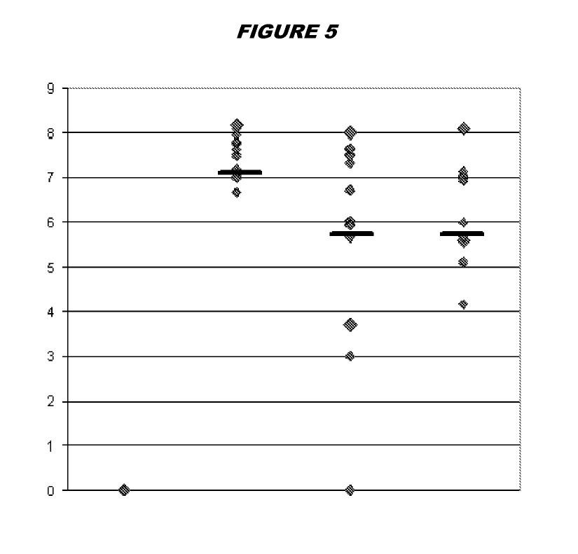

As shown in FIG. 4, a significant frequency of HtrA-specific CD4+−Th1+cells was elicited in mice immunized with the wild type and the mutant proteins. No significant differences in the CD4-Th1 response were found between the wild type and the mutant proteins, indicating that the H143R mutation does not interfere with the capability of the HtrA antigen to induce a CD4-Th1 response. Moreover, both the C. muridarum and C. trachomatis HtrA mutant proteins (TC210H143R and CT823H143R, respectively') were able to stimulate in vitro a specific CD4+ population cells that secretes IFN-γ in PBMC from mice immunized with the C. muridarum wild type and mutant HtrA. This indicates that the C. muridarum and C. trachomatis HtrA mutants are similar immunological properties.

### EXAMPLE 4

The protective activity of the wild-type and the mutant TC0210-(H143R) against C. trachomatis challenge in mice immunized with the recombinant proteins was evaluated in the mouse model.

Balb/c mice (15 mice per group) were immunized three times at two week intervals with TC0210 wild-type and TC0210 (H143R) mutant proteins using LTK63+CpG as adjuvant. A positive control was carried out in which the mice were immunized with 10³ live EBs. A negative control was also carded out in which the mice were immunized with the LTK63+CpG adjuvant only. Four weeks after the last immunization, the mice were challenged intranasally with 10³ IFU of infectious C. muridarum EBs. The protective activity of the wild type and mutant proteins was measured by measuring the presence of Chlamydial cells in the lung 10 days post-challenge. Specifically, IFU/lung was measured.

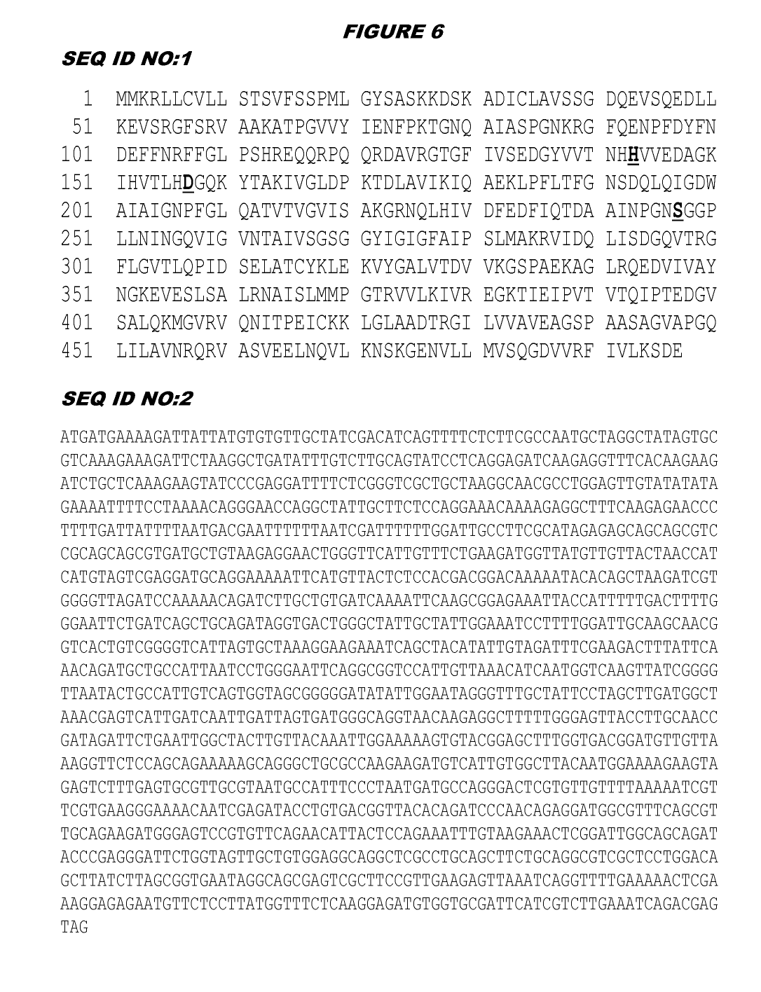

As shown in FIG. 5, the wild-type and mutant protein were able to reduce significantly the number of IFU/lung in challenged mice (approximately 1 log IFU reduction) as compared to adjuvant immunized mice.

The animal model results confirm that both the mice immunized with wild-type and mutant C. muridarum serine protease, give improved protection compared to immunisation with the adjuvant only. Further, the improvement in protection was similar for the wild type and mutant proteins.

These data confirm that, although the mutation in the catalytic site has inactivated the proteolytic activity of the protease, the protease conserves its immunological properties and so could be used alone or with other antigens in a vaccine composition against C. trachomatis infection.

It will be understood that the invention has been described by way of example only and modifications may be made whilst remaining within the scope and spirit of the invention.

TABLE 2

| C. pneumoniae accession number & annotation | C. trachomatis accession number & annotation | CT No. |
|---|---|---|
| | Hypothetical protein (AAC67968) | CT372 |
| | omcB (AAC68042) | CT443 |
| | Hypothetical protein (AAC67634) | CT043 |
| | Hypothetical protein (AAC67744) | CT153 |
| | Nqr3 (AAC67872) | CT279 |
| | papQ (AAC68203) | CT601 |
| | hypothetical protein (AAC68306) | CT711 |
| | hypothetical protein (AAC67705) | CT114 |
| | oppA_4 (AAC68080) | CT480 |
| | hypothetical protein (AAC68056) | CT456 |
| | ArtJ (AAC67977) | CT381 |
| | lcrE (AAC67680) | CT089 |
| | hypothetical protein (AAC68329) | CT734 |
| | hypothetical protein (AAC67606) | CT016 |
| gi\|4376729\|gb\|AAD18590.1\| Polymorphic Outer Membrane | gi\|3329346\|gb\|AAC68469.1\| Putative Outer Membrane Protein | |
| Protein G Family | G | |
| gi\|4376729\|gb\|AAD18590.1\| Polymorphic Outer Membrane | gi\|3329346\|gb\|AAC68469.1\| Putative Outer Membrane Protein | |
| Protein G Family | G | |
| gi\|4376731\|gb\|AAD18591.1\| Polymorphic Outer Membrane | gi\|3329346\|gb\|AAC68469.1\| Putative Outer Membrane Protein | |
| Protein G/I Family | | |
| gi\|4376731\|gb\|AAD18591.1\| Polymorphic Outer Membrane | gi\|3329350\|gb\|AAC68472.1\| Putative Outer Membrane Protein I | |
| Protein G/I Famliy | | |
| gi\|4376731\|gb\|AAD18591.1\| Polymorphic Outer Membrane | gi\|3329346\|gb\|AAC68469.1\| Putative Outer Membrane Protein | |
| Protein G/I Family | G | |
| gi\|4376733\|gb\|AAD18593.1\| Polymorphic Outer Membrane | gi\|3328840\|gb\|AAC68009.1\| Putative outer membrane protein A | |
| Protein G Family | | |
| gi\|4376731\|gb\|AAD18591.1\| Polymorphic Outer Membrane | gi\|3329346\|gb\|AAC68469.1\| Putative Outer Membrane Protein | |
| Protein G/I Famliy | G | |
| gi\|4376754\|gb\|AAD18011.1\| Polymorphic Outer Membrane | gi\|3329344\|gb\|AAC68467.1\| Putative Outer Membrane Protein | |
| Protein (Frame-shift with C | E | |
| gi\|4376260\|gb\|AAD18163.1\| Polymorphic Outer Membrane | gi\|3329346\|gb\|AAC68469.1\| Putative Outer Membrane Protein | |
| Protein G Family | G | |
| gi\|4376262\|gb\|AAD18165.1\| hypothetical protein | gi\|3328765\|gb\|AAC67940.1\| hypothetical protein | |
| gi\|4376269\|gb\|AAD18171.1\| hypothetical protein | gi\|3328825\|gb\|AAC67995.1\| hypothetical protein | |
| gi\|4376270\|gb\|AAD18172.1\| Polymorphic Outer Membrane | gi\|3329350\|gb\|AAC68472.1\| Putative Outer Membrane Protein | |
| Protein G Family | I | |
| gi\|4376272\|gb\|AAD181731\| Predicted OMP {leader peptide: | gi\|3328772\|gb\|AAC67946.1\| hypothetical protein | CT351 |
| outer membrane} | | |
| gi\|4376273\|gb\|AAD18174.1\| Predicted OMP {leader peptide} | gi\|3328771\|gb\|AAC67945.1\| hypothetical protein | CT350 |
| gi\|4376296\|gb\|AAD18195.1\| hypothetical protein | gi\|3328520\|gb\|AAC67712.1\| Ribulose-P Epimerase | |
| gi\|4376362\|gb\|AAD18254.1\|YbbP family hypothetical protein | gi\|3328401\|gb\|AAC67602.1\| hypothetical protein | |
| gi\|4376372\|gb\|AAD18263.1\| Signal Peptidase I | gi\|3328410\|gb\|AAC67610.1\| Signal Peptidase I | |
| gi\|4376397\|gb\|AAD18286.1\| CHLPS hypothetical protein | gi\|3328506\|gb\|AAC67700.1\| CHLPS hypotheticai protein | |
| gi\|4376402\|gb\|AAD18290.1\| ACR family | gi\|3328505\|gb\|AAC67699.1\| ACR family | |
| gi\|4376419\|gb\|AAD18305.1\| CT149 hypothetical protein | gi\|3328551\|gb\|AAC67740.1\| possible hydrolase | |
| gi\|4376446\|gb\|AAD18330.1\| hypothetical protein | gi\|3329261\|gb\|AAC68390.1\| hypothetical protein | |
| gi\|4376466\|gb\|AAD18348.1\| Oligopeptide Binding Protein | gi\|3328604\|gb\|AAC67790.1\| Oligopeptide Binding Protein | CT198 |
| gi\|4376467\|gb\|AAD18349.1\| Oligopeptide Binding Protein | gi\|3328604\|gb\|AAC67790.1\| Oligopeptide Binding Protein | |
| gi\|4376468\|gb\|AAD18350.1\| Oligopeptide Binding Protein | gi\|3328539\|gb\|AAC67730.1\| Oligopeptide Binding Protein | |
| gi\|4376469\|gb\|AAD18351.1\| Oligopeptide Binding Protein | gi\|3328579\|gb\|AAC67766.1\| Oligopeptide binding protein | |
| | permease | |
| gi\|4376520\|gb\|AAD18398.1\| Polysaccharide Hydrolase-invasin | gi\|3328526\|gb\|AAC67718.1\| predicted polysaccharide | |
| Repeat Family | hydrolase-invasin repeat family | |
| gi\|4376567\|gb\|AAD18441.1\| Inclusion Membrane Protein C | gi\|3328642\|gb\|AAC67825.1\| Inclusion Membrane Protein C | |
| gi\|4376576\|gb\|AAD18449.1\| Omp85 Analog | gi\|3328851\|gb\|AAC67834.1\| Omp85 Analog | CT241 |
| gi\|4376577\|gb\|AAD18450.1\| (OmpH-Like Outer Membrane | gi\|3328652\|gb\|AAC67835.1\| (OmpH-Like Outer Membrane | CT242 |
| Protein) | Protein) | |
| gi\|4376601\|gb\|AAD18472.1\| Low Calcium Response D | gi\|3328486\|gb\|AAC67681.1\| Low Calcium Response D | |
| gi\|4376602\|gb\|AAD18473.1\| Low Calcium Response E | gi\|3328485\|gb\|AAC67680.1\| Low Calcium Response E | CT089 |
| gi\|4376607\|gb\|AAD18478.1\| Phopholipase D Superfamily | gi\|3328479\|gb\|AAC67675.1\| Phopholipase D Superfamily | |
| | {leader (33) peptide} | |
| gi\|4376615\|gb\|AAD18485.1\| YojL hypothetical protein | gi\|3328472\|gb\|AAC67668.1\| hypothetical protein | CT077 |
| gi\|4376624\|gb\|AAD18493.1\| Solute Protein Binding Family | gi\|3328461\|gb\|AAC67658.1\| Solute Protein Binding Family | |
| gi\|4376639\|gb\|AAD18507.1\| Flagellar Secretion Protein | gi\|3328453\|gb\|AAC67651.1\| Flagellar Secretion Protein | |
| gi\|4376664\|gb\|AAD18529.1\| Leucyl Aminopeptidase A | gi\|3328437\|gb\|AAC67636.1\| Leucyl Aminopeptidase A | CT045 |
| gi\|4376672\|gb\|AAD18537.1\| CBS Domain protein (Hemolysin | gi\|3328667\|gb\|AAC67849.1\| Hypothetical protein containing | |
| Homolog) | CBS domains | |
| gi\|4376679\|gb\|AAD18543.1\| CT253 hypothetical protein | gi\|3328664\|gb\|AAC67846.1\| hypothetical protein | |
| gi\|4376696\|gb\|AAD18559.1\| CT266 hypothetical protein | gi\|3328678\|gb\|AAC67859.1\| hypothetical protein | CT266 |
| gi\|4376717\|gb\|AAD18579.1\| Phospholipase D superfamily | gi\|3328698\|gb\|AAC67877.1\| Phospholipase D superfamily | |
| gi\|4376727\|gb\|AAD18588.1\| Polymorphic Outer Membrane | gi\|3329346\|gb\|AAC68469.1\| Putative Outer Membrane Protein | |
| Protein G/I Family | G | |
| gi\|4376728\|gb\|AAD18589.1\| Polymorphic Outer Membrane | gi\|3329346\|gb\|AAC68469.1\| Putative Outer Membrane Protein | |
| Protein G Family | G | |
| gi\|4376729\|gb\|AAD18590.1\| Polymorphic Outer Membrane | gi\|3329350\|gb\|AAC68472.1\| Putative Outer Membrane Protein | |
| Protein G Family | I | |
| gi\|4376731\|gb\|AAD18591.1\| Polymorphic Outer Membrane | gi\|3329350\|gb\|AAC68472.1\| Putative Outer Membrane Protein | |
| Protein G/I Family | I | |
| gi\|4376733\|gb\|AAD18593.1\| Polymorphic Outer Membrane | gi\|3328840\|gb\|AAC68009.1\| Putative outer membrane protein A | |
| Protein G Family | | |
| gi\|4376735\|gb\|AAD18594.1\| Polymorphic Outer Membrane | gi\|3328840\|gb\|AAC68009.1\| Putative outer membrane protein A | |
| Protein (truncated) A/I Fern | | |
| gi\|4376736\|gb\|AAD18595.1\| Polymorphic Outer Membrane | gi\|3329346\|gb\|AAC68469.1\| Putative Outer Membrane Protein | |
| Protein G Family | G | |
| gi\|4376737\|gb\|AAD18596.1\| Polymorphic Outer Membrane | gi\|3329347\|gb\|AAC68470.1\| Putative Outer Membrane Protein | |
| Protein H Family | H | |
| gi\|4376751\|gb\|AAD18608.1\| Polymorphic Outer Membrane | gi\|3329344\|gb\|AAC68467.1\| Putative Outer Membrane Protein | |
| Protein E Family | E | |
| gi\|4376752\|gb\|AAD18609.1\| Polymorphic Outer Membrane | gi\|3329344\|gb\|AAC68467.1\| Putative Outer Membrane Protein | |
| Protein E Family | E | |
| gi\|4376753\|gb\|AAD18610.1\| Polymorphic Outer Membrane | gi\|3329344\|gb\|AAC68467.1\| Putative Outer Membrane Protein | |
| Protein E/F Family | E | |
| gi\|4376757\|gb\|AAD18613.1\| hypothetical protein | gi\|3328701\|gb\|AAC67880.1\| PP-loop superfamily ATPase | |
| gi\|4376767\|gb\|AAD18622.1\| Arginine Periplasmic Binding | gi\|3328806\|gb\|AAC67977.1\| Arginine Binding Protein | CT381 |
| Protein | | |
| gi\|4376790\|gb\|AAD18643.1\| Heat Shock Protein-70 | gi\|3328822\|gb\|AAC67993.1\| HSP-70 | CT396 |
| gi\|4376802\|gb\|AAD18654.1\| CT427 hypothetical protein | gi\|3328857\|gb\|AAC68024.1\| hypothetical protein | |
| gi\|4376814\|gb\|AAD18665.1\| CT398 hypothetical protein | gi\|3328825\|gb\|AAC67995.1\| hypothetical protein | CT398 |
| gi\|4376829\|gb\|AAD18679.1\| polymorphic membrane protein A | gi\|3328840\|gb\|AAC68009.1\| Putative outer membrane protein A | |
| Family | | |
| gi\|4376830\|gb\|AAD18680.1\| polymorphic membrane protein B | gi\|3328841\|gb\|AAC68010.1\| Putative outer membrane protein B | |
| Family | | |
| gi\|4376832\|gb\|AAD18681.1\| Solute binding protein | gi\|3328844\|gb\|AAC68012.1\| Solute-binding protein | CT415 |
| gi\|4376834\|gb\|AAD18683.1\| (Metal Transport Protein) | gi\|3328846\|gb\|AAC68014.1\| (Metal Transport Protein) | |
| gi\|4376847\|gb\|AAD18695.1\| Tail-Specific Protease | gi\|3328872\|gb\|AAC68040.1\| Tail-Specific Protease | |
| gi\|4376848\|gb\|AAD18696.1\| 15 kDa Cysteine-Rich Protein | gi\|3328873\|gb\|AAC68041.1\| 15 kDa Cysteine-Rich Protein | |
| gi\|4376849\|gb\|AAD18697.1\| 60 kDa Cysteine-Rich OMP | gi\|3328874\|gb\|AAC68042.1\| 60 kDa Cysteine-Rich OMP | CT443 |
| gi\|4376850\|gb\|AAD18698.1\| 9 kDa-Cysteine-Rich Lipoprotein | gi\|3328876\|gb\|AAC68043.1\| 9 kDa-Cysteine-Rich Lipoprotein | CT444 |
| gi\|4376878\|gb\|AAD18723.1\| 2-Component Sensor | gi\|3328901\|gb\|AAC68067.1\| 2-component regulatory system- | CT467 |
| | sensor histidine kinase | |
| gi\|4376879\|gb\|AAD18724.1\| similarity to CHLPS IncA | gi\|3328451\|gb\|AAC67649.1\| hypothetical protein | |
| gi\|4376884\|gb\|AAD18729.1\| CT471 hypothetical protein | gi\|3328905\|gb\|AAC68071.1\| hypothetical protein | |
| gi\|4376886\|gb\|AAD18731.1\| YidD family | gi\|3328908\|gb\|AAC68073.1\| hypothetical protein | |
| gi\|4376890\|gb\|AAD18734.1\| CT476 hypothetical protein | gi\|3328911\|gb\|AAC68076.1\| hypothetical protein | |
| gi\|4376892\|gb\|AAD18736.1\| Oligopeptide Permease | gi\|3328913\|gb\|AAC68078.1\| Oligopeptide Permease | |
| gi\|4376894\|gb\|AAD18738.1\| Oligopeptide Binding Lipoprotein | gi\|3328915\|gb\|AAC68080.1\| Oligopeptide Binding Lipoprotein | |
| gi\|4376900\|gb\|AAD18743.1\| Glutamine Binding Protein | gi\|3328922\|gb\|AAC68086.1\| Glutamine Binding Protein | |
| gi\|4376909\|gb\|AAD18752.1\| Protease | gi\|6578107\|gb\|AAC68094.2\| Protease | |
| gi\|4376952\|gb\|AAD18792.1\| Apolipoprotein N-Acetyltrans- | gi\|3328972\|gb\|AAC68136.1\| Apolipoprotein N-Acetyltrans- | |
| ferase | ferase | |
| gi\|4376960\|gb\|AAD18800.1\| FKBP-type peptidyl-prolyl cis- | gi\|3328979\|gb\|AAC68143.1\| FKBP-type peptidyl-prolyl cis- | CT541 |
| trans isomerase | trans isomerase | |
| gi\|4376968\|gb\|AAD18807.1\| CT547 hypothetical protein | gi\|3328986\|gb\|AAC68149.1\| hypothetical protein | CT547 |
| gi\|4376969\|gb\|AAD18808.1\| CT548 hypothetical protein | gi\|3328987\|gb\|AAC68150.1\| hypothetical protein | |
| gi\|4376998\|gb\|AAD18834.1\| Major Outer Membrane Protein | gi\|3329133\|gb\|AAC68276.1\| Major Outer Membrane Protein | CT681 |
| gi\|4377005\|gb\|AAD18841.1\| YopC/Gen Secretion Protein D | gi\|3329125\|gb\|AAC68269.1\| probable Yop proteins | |
| | translocation protein | |
| gi\|4377015\|gb\|AAD18851.1\| FHA domain; (homology to | gi\|3329115\|gb\|AAC68259.1\| FHA domain; homology to | |
| adenylate cyclase) | adenylate cyclase) | |
| gi\|4377033\|gb\|AAD18867.1\| CHLPN 76 kDA Homolog_1 | gi\|3329069\|gb\|AAC68226.1\| CHLPN 76 kDa Homolog | CT622 |
| (CT622) | | |
| gi\|4377034\|gb\|AAD18868.1\| CHLPN 76 kDa Homolog_2 | gi\|6578109\|gb\|AAC68227.2\| CHLPN 76 kDa Homolog | CT623 |
| (CT623) | | |
| gi\|4377035\|gb\|AAD18869.1\| Integral Membrane Protein | gi\|3329071\|gb\|AAC68228.1\| Integral Membrane Protein | |
| gi\|4377072\|gb\|AAD18902.1\| CT648 hypothetical protein | gi\|3329097\|gb\|AAC68825.1\| hypothetical protein | |
| gi\|4377073\|gb\|AAD18903.1\| CT647 hypothetical protein | gi\|3329096\|gb\|AAC68824.1\| hypothetical protein | CT647 |
| gi\|4377085\|gb\|AAD18914.1\| CT605 hypothetical protein | gi\|3329050\|gb\|AAC68208.1\| hypothetical protein | |
| gi\|4377090\|gb\|AAD18919.1\| Peptidoglycan-Associated | gi\|3329044\|gb\|AAC68202.1\| Peptidoglycan-Associated | CT600 |
| Lipoprotein | Lipoprotein | |
| gi\|4377091\|gb\|AAD18920.1\| macromolecule transporter | gi\|3329043\|gb\|AAC68201.1\| component of a macromolecule | |
| | transport system | |
| gi\|4377092\|gb\|AAD18921.1\| CT598 hypothetical protein | gi\|3329042\|gb\|AAC68200.1\| hypothetical protein | |
| gi\|4377093\|gb\|AAD18922.1\| Biopolymer Transport Protein | gi\|3329041\|gb\|AAC68199.1\| Biopolymer Transport Protein | CT597 |
| gi\|4377094\|gb\|AAD18923.1\| Macromolecule transporter | gi\|3329040\|gb\|AAC68198.1\| polysaccharide transporter | |
| gi\|4377101\|gb\|AAD18929.1\| CT590 hypothetical protein | gi\|3329033\|gb\|AAC68192.1\| hypothetical protein | |
| gi\|4377102\|gb\|AAD18930.1\| CT589 hypothedcal protein | gi\|3329032\|gb\|AAC68191.1\| hypothetical protein | CT589 |
| gi\|4377106\|gb\|AAD18933.1\| hypothetical protein | gi\|3328796\|gb\|AAC67968.1\| hypothetical protein | |
| gi\|4377111\|gb\|AAD18938.1\| Enolase | gi\|3329030\|gb\|AAC68189.1\| Enolase | CT587 |
| gi\|4377127\|gb\|AAD18953.1\| General Secretion Protein D | gi\|3329013\|gb\|AAC68174.1\| Gen. Secretion Protein D | |
| gi\|4377130\|gb\|AAD18956.1\| predicted OMP {leader peptide} | gi\|3329010\|gb\|AAC68171.1\| predicted OMP | CT569 |
| gi\|4377132\|gb\|AAD18958.1\| CT567 hypothetical protein | gi\|3329008\|gb\|AAC68169.1\| hypothetical protein | CT567 |
| gi\|4377133\|gb\|AAD18959.1\| CT566 hypothetical protein | gi\|3329007\|gb\|AAC68168.1\| hypothetical protein | |
| gi\|4377140\|gb\|AAD18965.1\| Yop Translocation J | gi\|3329000\|gb\|AAC68161.1\| Yop proteins translocation | CT559 |
| | lipoprotein J | |
| gil4377170\|gb\|AAD18992.1\| Outer Membrane Protein B | gi\|3329169\|gb\|AAC68308.1\| Outer Membrane Protein Analog | CT713 |
| gi\|4377177\|gb\|AAD18998.1\| Flagellar M-Ring Protein | gi\|3329175\|gb\|AAC68314.1\| Flagellar M-Ring Protein | |
| gi\|4377182\|gb\|AAD19003.1\| CT724 hypothetical protein | gi\|3329181\|gb\|AAC68319.1\| hypothetical protein | |
| gi\|4377184\|gb\|AAD19005.1\| Rod Shape Protest | gi\|3329183\|gb\|AAC68321.1\| Rod Shape Protein | |
| gi\|4377193\|gb\|AAD19013.1\| CT734 hypothetical protein | gi\|3329192\|gb\|AAC68329.1\| hypothetical protein | |
| gi\|4377206\|gb\|AAD19025.1\| CHLTR possible phosphoprotein | gi\|3329204\|gb\|AAC68339.1\| CHLTR possible phosphoprotein | |
| gi\|4377222\|gb\|AAD19040.1\| Muramidase (invasin repeat family) | gi\|3329221\|gb\|AAC68354.1\| Muramidase (invasin repeat family) | CT759 |
| gi\|4377223\|gb\|AAD19041.1\| Cell Division Protein FtsW | gi\|3329222\|gb\|AAC68355.1\| Cell Division Protein FtsW | |
| gi\|4377224\|gb\|AAD19042.1\| Peptidoglycan Transferase | gi\|3329223\|gb\|AAC68356.1\| Peptidoglycan Transferase | CT761 |
| gi\|4377225\|gb\|AAD19043.1\| Murarnate-Ala Ligase & D-Ala- | gi\|3329224\|gb\|AAC68357.1\| UDP-N-acetylmuramate-alanine | |
| D-Ala Ligase | ligase | |
| gi\|4377248\|gb\|AAD19064.1\| Thioredoxin Disulfide, Isomerase | gi\|3329244\|gb\|AAC68375.1\| Thioredoxin Disulfide Isomerase | |
| gi\|4377261\|gb\|AAD19076.1\| CT788 hypothetical protein- | gi\|3329253\|gb\|AAC68383.1\| {leader (60) peptide-periplasmic} | |
| {leader peptide-periplasmi | | |
| gi\|4377280\|gb\|AAD19093.1\| Insulinase family/Protease III | gi\|3329273\|gb\|AAC68402.1\| Insulinase family/Protease III | |
| gi\|4377287\|gb\|AAD19099.1\| Putative Outer Membrane Protein | gi\|3329279\|gb\|AAC68408.1\| Putative Outer Membrane Protein | |
| D Family | D | |
| gi\|4377306\|gb\|AAD19116.1\| DO Serine Protease | gi\|3329293\|gb\|AAC68420.1\| DO Serine Protease | CT823 |
| gi\|4377342\|gb\|AAD19149.1\| ABC transporter permease | gi\|3329327\|gb\|AAC68451.1\| ABC transporter permease- | |
| | pyrimidine biosynthesis protein | |
| gi\|4377347\|gb\|AAD19153.1\| CT858 hypothetical protein | gi\|6578118\|gb\|AAC68456.2\| predicted Protease containing | |
| | IRBP and DHR domains | |
| gi\|4377353\|gb\|AAD19159.1\| CT863 hypothetical protein | gi\|3329337\|gb\|AAC68461.1\| hypothetical protein | |
| gi\|4377367\|gb\|AAD19171.1\| Predicted OMP | gi\|3328795\|gb\|AAC67967.1\| hypothetical protein | |
| gi\|4377408\|gb\|AAD19209.1\| hypothetical protein | gi\|3328795\|gb\|AAC67967.1\| hypothetical protein | |
| gi\|4377409\|gb\|AAD19210.1\| Predicted Outer Membrane Pro- | gi\|3328795\|gb\|AAC67967.1\| hypothetical protein | |
| tein (CT371) | | |
| gi\|4376411\|gb\| | gi\|3328512\|gb\|AAC67705.1\| hypothetical protein | CT114 |
| gi\|4376508\|gb\| | gi\|3328585\|gb\|AAC67772.1\| hypothetical protein | CT181 |
| gi\|4376710\|gb\| | gi\|3328692\|gb\|AAC67872.1\| NADH (Ubiquinone) | CT279 |
| | Oxidoreductase, Gamma | |
| gi\|4376777\|gb\| | gi\|3328815\|gb\|AAC67986.1\| hypothetical protein | CT389 |
| gi\|4376782\|gb\| | gi\|3328817\|gb\|AAC67988.1\| hypothetical protein | CT391 |
| gi\|4376863\|gb\| | gi\|3328887\|gb\|AAC68054.1\| Arginyl tRNA transferase | CT454 |
| gi\|4376866\|gb\| | gi\|3328889\|gb\|AAC68056.1\| hypothetical protein | CT456 |
| gi\|4376972\|gb\| | gi\|3328991\|gb\|AAC68153.1\| D-Ala-D-Ala Carboxypeptidase | CT551 |
| gi\|4377139\|gb\| | gi\|3329001\|gb\|AAC68162.1\| hypothetical protein | CT560 |
| gi\|4377154\|gb\| | gi\|3329154\|gb\|AAC68295.1\| hypothetical protein | CT700 |
| gi\|4377191\|gb\|AAD19012.1\| hypothetical protein | gi\|3329191\|gb\|AAC68328.1\| hypothetical protein | CT733 |

1. A method of treating, preventing or diagnosing Chlamydia in a patient, comprising administering a therapeutically effective amount of:
(a) an immunogenic Chlamydia HtrA protein which has one or more mutations relative to wild-type Chlamydia HtrA that result in a reduced or eliminated protease activity relative to the protease activity of wild-type Chlamydia HtrA;
(b) a fragment of (a) which comprises 50 or more consecutive amino acids from the protein and comprises the one or more mutations that result in the reduced or eliminated protease activity, and wherein the fragment is capable of eliciting an immune response against the wild-type Chlamydia HtrA protein;
(c) an antibody which binds (a) or (b) but which does not bind to wild-type HtrA protein; or
(d) a nucleic acid that encodes (a) or (b).

2. The method of claim 1 wherein the immunogenic Chlamydia HtrA protein comprises SEQ ID NO:4.

3. The method of claim 1, wherein the wild-type HtrA is from C. trachomatis.

4. The method of claim 1, wherein the wild-type HtrA comprises the sequence of SEQ ID NO:1.

5. The method of claim 1, wherein the one or more mutations are each independently a substitution, an insertion or a deletion.

6. The method of claim 1, wherein at least one of the one or more mutations is selected from the group consisting of the substitution of a histidine (H) with a glycine (G), alanine (A), serine (S), valine (V) or threonine (T), lysine (K), glutamine (Q) or an asparagine (N); substitution of an aspartatic acid (D) with a threonine, serine, valine, glycine, alanine or glutamic acid (E); substitution of a serine (S) with a glycine (G), valine (V), asparagine (N) or aspartic acid (D), alanine (A) or a threonine (T).

7. The method of claim 1, wherein at least one of the one or more mutations is in the protease domain.

8. The method of claim 1, wherein at least one of the one or more mutations is of a residue in the catalytic triad of histidine, aspartate and serine.

9. The method of claim 8, wherein the histidine of the catalytic triad is mutated to arginine.

10. The method of claim 1, wherein at least one of the one or more mutations is of a residue in close proximity to the residues of the catalytic triad in the three dimensional conformation of the Chlamydia HtrA protein or is of a residue that is conserved across the HtrA protein family.

11. The method of claim 1, wherein the wild-type HtrA is from C. trachomatis and at least one of the one or more mutations is H142R or H143R.

12. The method of claim 1, wherein the immunogenic Chlamydia HtrA protein comprises the sequence SEQ ID NO:5.

13. The method of claim 1, wherein the reduced or eliminated protease activity is conferred by a single mutation.


SEQUENCE LISTING (70 sequences)

SEQ ID NO: 1 - protein, 497 aa, Chlamydia trachomatis
MMKRLLCVLLSTSVFSSPMLGYSASKKDSKADICLAVSSGDQEVSQEDLLKEVSRGFSRVAAKATPGVVYIENFPKTGNQAIASPGNKRGFQENPFDYFNDEFFNRFFGLPSHREQQRPQQRDAVRGTGFIVSEDGYVVTNHHVVEDAGKIHVTLHDGQKYTAKIVGLDPKTDLAVIKIQAEKLPFLTFGNSDQLQIGDWAIAIGNPFGLQATVTVGVISAKGRNQLHIVDFEDFIQTDAAINPGNSGGPLLNINGQVIGVNTAIVSGSGGYIGIGFAIPSLMAKRVIDQLISDGQVTRGFLGVTLQPIDSELATCYKLEKVYGALVTDVVKGSPAEKAGLRQEDVIVAYNGKEVESLSALRNAISLMMPGTRVVLKIVREGKTIEIPVTVTQIPTEDGVSALQKMGVRVQNITPEICKKLGLAADTRGILVVAVEAGSPAASAGVAPGQLILAVNRQRVASVEELNQVLKNSKGENVLLMVSQGDVVRFIVLKSDE

SEQ ID NO: 2 - DNA, 1494 bp, Chlamydia trachomatis
atgatgaaaagattattatgtgtgttgctatcgacatcagttttctcttcgccaatgctaggctatagtgcgtcaaagaaagattctaaggctgatatttgtcttgcagtatcctcaggagatcaagaggtttcacaagaagatctgctcaaagaagtatcccgaggattttctcgggtcgctgctaaggcaacgcctggagttgtatatatagaaaattttcctaaaacagggaaccaggctattgcttctccaggaaacaaaagaggctttcaagagaacccttttgattattttaatgacgaattttttaatcgattttttggattgccttcgcatagagagcagcagcgtccgcagcagcgtgatgctgtaagaggaactgggttcattgtttctgaagatggttatgttgttactaaccatcatgtagtcgaggatgcaggaaaaattcatgttactctccacgacggacaaaaatacacagctaagatcgtggggttagatccaaaaacagatcttgctgtgatcaaaattcaagcggagaaattaccatttttgacttttgggaattctgatcagctgcagataggtgactgggctattgctattggaaatccttttggattgcaagcaacggtcactgtcggggtcattagtgctaaaggaagaaatcagctacatattgtagatttcgaagactttattcaaacagatgctgccattaatcctgggaattcaggcggtccattgttaaacatcaatggtcaagttatcggggttaatactgccattgtcagtggtagcgggggatatattggaatagggtttgctattcctagcttgatggctaaacgagtcattgatcaattgattagtgatgggcaggtaacaagaggctttttgggagttaccttgcaaccgatagattctgaattggctacttgttacaaattggaaaaagtgtacggagctttggtgacggatgttgttaaaggttctccagcagaaaaagcagggctgcgccaagaagatgtcattgtggcttacaatggaaaagaagtagagtctttgagtgcgttgcgtaatgccatttccctaatgatgccagggactcgtgttgttttaaaaatcgttcgtgaagggaaaacaatcgagatacctgtgacggttacacagatcccaacagaggatggcgtttcagcgttgcagaagatgggagtccgtgttcagaacattactccagaaatttgtaagaaactcggattggcagcagatacccgagggattctggtagttgctgtggaggcaggctcgcctgcagcttctgcaggcgtcgctcctggacagcttatcttagcggtgaataggcagcgagtcgcttccgttgaagagttaaatcaggttttgaaaaactcgaaaggagagaatgttctccttatggtttctcaaggagatgtggtgcgattcatcgtcttgaaatcagacgagtag

SEQ ID NO: 3 - protein, 497 aa, Chlamydia muridarum
MMKRLLCVLLSTSVFSSPMLGYSAPKKDSSTGICLAASQSDRELSQEDLLKEVSRGFSKVAAQATPGVVYIENFPKTGSQAIASPGNKRGFQENPFDYFNDEFFNRFFGLPSHREQPRPQQRDAVRGTGFIVSEDGYVVTNHHVVEDAGKIHVTLHDGQKYTAKIIGLDPKTDLAVIKIQAKNLPFLTFGNSDQLQIGDWSIAIGNPFGLQATVTVGVISAKGRNQLHIVDFEDFIQTDAAINPGNSGGPLLNIDGQVIGVNTAIVSGSGGYIGIGFAIPSLMAKRVIDQLISDGQVTRGFLGVTLQPIDSELAACYKLEKVYGALITDVVKGSPAEKAGLRQEDVIVAYNGKEVESLSALRNAISLMMPGTRVVLKVVREGKFIEIPVTVTQIPAEDGVSALQKMGVRVQNLTPEICKKLGLASDTRGIFVVSVEAGSPAASAGVVPGQLILAVNRQRVSSVEELNQVLKNAKGENVLLMVSQGEVIRFVVLKSDE

SEQ ID NO: 4 - protein, 497 aa, Chlamydia trachomatis
MMKRLLCVLLSTSVFSSPMLGYSASKKDSKADICLAVSSGDQEVSQEDLLKEVSRGFSRVAAKATPGVVYIENFPKTGNQAIASPGNKRGFQENPFDYFNDEFFNRFFGLPSYREQQRPQQRDAVRGTGFIVSEDGYVVTNHHVVEDAGKIHVTLHDGQKYTAKIVGLDPKTDLAVIKIQAEKLPFLTFGNSDQLQIGDWAIAIGNPFGLQATVTVGVVSAKGRNQLHIVDFEDFIQTDAAINPGNAGGPLLNINGQVIGVNTAIVSGSGGYIGIGFAIPSLMAKRVIDQLISDGQVTRGFLGVTLQPIDSELATCYKLEKVYGALVTDVVKGSPAEKAGLRQEDVIVAYNGKEVESLSALRNAISLMMPGTRVILKIVREGKTIEIPVTVTQIPTEDGVSALQKMGVRVQNITPEICKKLGLAADTRGILVVAVEAGSPAASAGVAPGQLILAVNRQRVASVEELNQVLKNSKGENVLLMVSQGDVVRFIVLKSDE

SEQ ID NO: 5 - protein, 497 aa, Chlamydia trachomatis
MMKRLLCVLLSTSVFSSPMLGYSASKKDSKADICLAVSSGDQEVSQEDLLKEVSRGFSRVAAKATPGVVYIENFPKTGNQAIASPGNKRGFQENPFDYFNDEFFNRFFGLPSHREQQRPQQRDAVRGTGFIVSEDGYVVTNHRVVEDAGKIHVTLHDGQKYTAKIVGLDPKTDLAVIKIQAEKLPFLTFGNSDQLQIGDWAIAIGNPFGLQATVTVGVISAKGRNQLHIVDFEDFIQTDAAINPGNSGGPLLNINGQVIGVNTAIVSGSGGYIGIGFAIPSLMAKRVIDQLISDGQVTRGFLGVTLQPIDSELATCYKLEKVYGALVTDVVKGSPAEKAGLRQEDVIVAYNGKEVESLSALRNAISLMMPGTRVVLKIVREGKTIEIPVTVTQIPTEDGVSALQKMGVRVQNITPEICKKLGLAADTRGILVVAVEAGSPAASAGVAPGQLILAVNRQRVASVEELNQVLKNSKGENVLLMVSQGDVVRFIVLKSDE

SEQ ID NO: 6 - protein, 442 aa, Chlamydia trachomatis
MQAAHHHYHRYTDKLHRQNHKKDLISPKPTEQEACNTSSLSKELIPLSEQRGLLSPICDFISERPCLHGVSVRNLKQALKNSAGTQIALDWSILPQWFNPRVSHAPKLSIRDFGYSAHQTVTEATPPCWQNCFNPSAAVTIYDSSYGKGVFQISYTLVRYWRENAATAGDAMMLAGSINDYPSRQNIFSQFTFSQNFPNERVSLTIGQYSLYAIDGTLYNNDQQLGFISYALSQNPTATYSSGSLGAYLQVAPTASTSLQIGFQDAYNISGSSIKWSNLTKNRYNFHGFASWAPRCCLGSGQYSVLLYVTRQVPEQMEQTMGWSVNASQHISSKLYVFGRYSGVTGHVFPINRTYSFGMASANLFNRNPQDLFGIACAFNNVHLSASPNTKRKYETVIEGFATIGCGPYLSFAPDFQLYLYPALRPNKQSARVYSVRANLAI

SEQ ID NO: 7 - protein, 553 aa, Chlamydia trachomatis
MRIGDPMNKLIRRAVTIFAVTSVASLFASGVLETSMAESLSTNVISLADTKAKDNTSHKSKKARKNHSKETPVDRKEVAPVHESKATGPKQDSCFGRMYTVKVNDDRNVEITQAVPEYATVGSPYPIEITATGKRDCVDVIITQQLPCEAEFVRSDPATTPTADGKLVWKIDRLGQGEKSKITVWVKPLKEGCCFTAATVCACPEIRSVTKCGQPAICVKQEGPENACLRCPVVYKINIVNQGTATARNVVVENPVPDGYAHSSGQRVLTFTLGDMQPGEHRTITVEFCPLKRGRATNIATVSYCGGHKNTASVTTVINEPCVQVSIAGADWSYVCKPVEYVISVSNPGDLVLRDVVVEDTLSPGVTVLEAAGAQISCNKVVWTVKELNPGESLQYKVLVRAQTPGQFTNNVVVKSCSDCGTCTSCAEATTYWKGVAATHMCVVDTCDPVCVGENTVYRICVTNRGSAEDTNVSLMLKFSKELQPVSFSGPTKGTITGNTVVFDSLPRLGSKETVEFSVTLKAVSAGDARGEAILSSDTLTVPVSDTENTHIY

SEQ ID NO: 8 - protein, 167 aa, Chlamydia trachomatis
MSRQNAEENLKNFAKELKLPDVAFDQNNTCILFVDGEFSLHLTYEEHSDRLYVYAPLLDGLPDNPQRRLALYEKLLEGSMLGGQMAGGGVGVATKEQLILMHCVLDMKYAETNLLKAFAQLFIETVVKWRTVCSDISAGREPTVDTMPQMPQGGGGGIQPPPAGIRA

SEQ ID NO: 9 - protein, 810 aa, Chlamydia trachomatis
MTKPSFLYVIQPFSVFNPRLGRFSTDSDTYIEEENRLASFIESLPLEIFDIPSFMETAISNSPYILSWETTKDGALFTILEPKLSACAATCLVAPSIQMKSDAELLEEIKQALLRSSHDGVKYRITRESFSPEKKTPKVALVDDDIELIRNVDFLGRAVDIVKLDPINILNTVSEENILDYSFTRETAQLSADGRFGIPPGTKLFPKPSFDVEISTSIFEETTSFTRSFSASVTFSVPDLAATMPLQSPPMVENGQKEICVIQKHLFPSYSPKLVDIVKRYKREAKILINKLAFGMLWRHRAKSQILTEGSVRLDLQGFTESKYNYQIQVGSHTIAAVLIDMDISKIQSKSEQAYAIRKIKSGFQRSLDDYHIYQIERKQTFSFSPKHRSLSSTSHSEDSDLDLSEAAAFSGSLTCEFVKKSTQHAKNTVTCSTAAHSLYTLKEDDSSNPSEKRLDSCFRNWIENKLSANSPDSWSAFIQKFGTHYIASATFGGIGFQVLKLSFEQVEDLHSKKISLETAAANSLLKGSVSSSTESGYSSYSSTSSSHTVFLGGTVLPSVHDERLDFKDWSESVHLEPVPIQVSLQPITNLLVPLHFPNIGAAELSNKRESLQQAIRVYLKEHKVDEQGERTTFTSGIDNPSSWFTLEAAHSPLIVSTPYIASWSTLPYLFPTLRERSSATPIVFYFCVDNNEHASQKILNQSYCFLGSLPIRQKIFGSEFASFPYLSFYGNAKEAYFDNTYYPTRCGWIVEKLNTTQDQFLRDGDEVRLKHVSSGKYLATTPLKDTHGTLTRTTNCEDAIFIIKKSSGY

SEQ ID NO: 10 - protein, 316 aa, Chlamydia trachomatis
MASKSRHYLNQPWYIILFIFVLSLIAGTLLSSVYYVLAPIQQQAAEFDRNQQMLMAAQVISSDNTFQVYEKGDWHPALYNTKKQLLEISSTPPKVTVTTLSSYFQNFVRVLLTDTQGNLSSFEDHNLNLEEFLSQPTPVIHGLALYVVYAILHNDAASSKLSASQVAKNPTAIESIVLPIEGFGLWGPIYGFLALEKDGNTVLGTSWYQHGETPGLGANIANPQWQKNFRGKKVFLVSASGETDFAKTTLGLEVIKGSVSAALGDSPKAASSIDGISGATLTCNGVTESFSHSLAPYRALLTFFANSKPSGESHDH

SEQ ID NO: 11 - protein, 200 aa, Chlamydia trachomatis
MLANRLFLITLLGLSSSVYGAGKAPSLQAILAEVEDTSSRLHAHHNELAMISERLDEQDTKLQQLSSTQDHNLPRQVQRLETDQKALAKTLAILSQSVQDIRSSVQNKLQEIQQEQKKLAQNLRALRNSLQALVDGSSPENYIDFLTGETPEHIHIVKQGETLSKIASKYNIPVVELKKLNKLNSDTIFTDQRIRLPKKK

SEQ ID NO: 12 - protein, 767 aa, Chlamydia trachomatis
MSIQPTSISLTKNITAALAGEQVDAAAVYMPQAVFFFQQLDEKSKGLKQALGLLEEVDLEKFIPSLEKSPTPITTGTTSKISADGIEIVGELSSETILADPNKAAAQVFGEGLADSFDDWLRLSENGGIQDPTAIEEEIVTKYQTELNTLRNKLKQQSLTDDEYTKLYAIPQNFVKEIESLKNENNVRLIPKSKVTNFWQNIMLTYNSVTSLSEPVTDAMNTTMAEYSLYIERATEAAKLIREITNTIKDIFNPVWDVREQTGIFGLKGAEYNALEGNMIQSLLSFAGLFRQLMSRTATVDEIGALYPKNDKNEDVIHTAIDDYVNSLADLKANEQVKLNGLLSLVYAYYASTLGFAKKDVFNNAQASFTDYTNFLNQEIQYWTPRETSSFNISNQALQTFKNKPSADYNGVYLFDNKGLETNLFNPTFFFDVVSLMTADPTKTMSRQDYNKVITASESSIQKINQAITAWELAIAECGTKKAKLEPSSLNYFNAMVEAKKTFVETSPIQMVYSSLMLDKYLPNQQYILETLGSQMTFSNKAARYLNDIIAYAVSFQTADVYYSLGMYLRQMNQQEFPEVISRANDTVKKEIDRSRADLFHCKKAIEKIKELVTSVNADTELTSSQRAELLETLASYAFEFENLYHNLSNVYVMVSKVQISGVSKPDEVDEAFTAKIGSKEFDTWIQQLTTFESAVIEGGRNGVMPGGEQQVLQSLESKQQDYTSFNQNQQLALQMESAAIQQEWTMVAAALALMNQIFAKLIRRFK

SEQ ID NO: 13 - protein, 486 aa, Chlamydia trachomatis
MCFIGIGSLLLPTALRATERMRKEPIPLLDKQQSFWNVDPYCLESICACFVAHRDPLSAKQLMYLFPQLSEEDVSVFARCILSSKRPEYLFSKSEEELFAKLILPRVSLGVHRDDDLARVLVLAEPSAEEQKARYYSLYLDVLALRAYVERERLASAAHGDPERIDLATIEAINTILFQEEGWRYPSKQEMFENRFSELAAVTDSKFGVCLGTVVLYQAVAQRLDLSLDPVTPPGHIYLRYKDKVNIETTSGGRHLPTERYCECIKESQLKVRSQMELIGLTFMNRGAFFLQKGEFLQASLAYEQAQSYLSDEQISDLLGITYVLLGKKAAGEALLKKSAEKTRRGSSIYDYFQGYISPEILGVLFADSGVTYQETLEYRKKLVMLSKKYPKSGSLRLRLATTALELGLVKEGVQLLEESVKDAPEDLSLRLQFCKILCNRHDYVRAKYHFDQAQALLIKEGLFSEKTSYTLLKTIGKKLSLFAPS

SEQ ID NO: 14 - protein, 696 aa, Chlamydia trachomatis
MIDKIIRTILVLSLFLLYWSSDLLEKDVKSIKRELKALHEDVLELVRISHQQKNWVQSTDFSVSPEISVLKDCGDPAFPNLLCEDPYVEKVVPSLLKEGFVPKGILRTAQVGRPDNLSPFNGFVNIVRFYELCVPNLAVEHVGKYEEFAPSLALKIEEHYVEDGSGDKEFHIYLRPNMFWEPIDPTLFPKNITLADSFLRPHPVTAHDVKFYYDVVMNPYVAEMRAVAMRSYFEDMVSVRVENDLKLIVRWRAHTVRNEQGEEEKKVLYSAFANTLALQPLPCFVYQHFANGEKIVPEDSDPDTYRKDSVWAQNFSSHWAYNYIVSCGAFRFAGMDDEKITLVRNPNYHNPFAALVEKRYIYMKDSTDSLFQDFKAGKVDIAYFPPNHVDNLASFMQTSAYKEQAARGEAILEKNSSDRSYSYIGWNCLSLFFNNRSVRQAMNMLIDRDRIIEQCLDGRGVSVSGPFSLCSPSYNRDVEGWQYSPEEAARKLEEEGWIDADGDGIREKVIDGVVVPFRFRLCYYVKSVTARTIAEYVATVCKEVGIECCLLGLDMADYSQALEEKNFDAILSGWCLGTPPEDPRALWHSEGALEKGSANAVGFCNEEADRIIEQLSYEYDSNKRQALYHRFHEVIHEESPYAFLYSRQYSLVYKEFVKNIFVPTEHQDLIPGAQDETVNLSMLWVDKEEGRCSAIS

SEQ ID NO: 15 - protein, 1005 aa, Chlamydia trachomatis
MTNSISGYQPTVTTSTSSTTSASGASGSLGASSVSTTANATVTQTANATNSAATSSIQTTGETVVNYTNSASAPNVTVSTSSSSTQATATSNKTSQAVAGKITSPDTSESSETSSTSSSDHIPSDYDDVGSNSGDISNNYDDVGSNNGDISSNYDDAAADYEPIRTTENIYESIGGSRTSGPENTSGGAAAALNSLRGSSYSNYDDAAADYEPIRTTENIYESIGGSRTSGPENTSGGAAAALNSLRGSSYSNYDDAAADYEPIRTTENIYESIGGSRTSGPENTSDGAAAAALNSLRGSSYTTGPRNEGVFGPGPEGLPDMSLPSYDPTNKTSLLTFLSNPHVKSKMLENSGHFVFIDTDRSSFILVPNGNWDQVCSIKVQNGKTKEDLDIKDLENMCAKFCTGFSKFSGDWDSLVEPMVSAKAGVASGGNLPNTVIINNKFKTCVAYGPWNSQEASSGYTPSAWRRGHRVDFGGIFEKANDFNKINWGTQAGPSSEDDGISFSNETPGAGPAAAPSPTPSSIPIINVNVNVGGTNVNIGDTNVNTTNTTPTTQSTDASTDTSDIDDINTNNQTDDINTTDKDSDGAGGVNGDISETESSSGDDSGSVSSSESDKNASVGNDGPAMKDILSAVRKHLDVVYPGENGGSTEGPLPANQTLGDVISDVENKGSAQDTKLSGNTGAGDDDPTTTAAVGNGAEEITLSDTDSGIGDDVSDTASSSGDESGGVSSPSSESNKNTAVGNDGPSGLDILAAVRKHLDKVYPGDNGGSTEGPLQANQTLGDIVQDMETTGTSQETVVSPWKGSTSSTESAGGSGSVQTLLPSPPPTPSTTTLRTGTGATTTSLMMGGPIKADIITTGGGGRIPGGGTLEKLLPRIRAHLDISFDAQGDLVSTEEPQLGSIVNKFRQETGSRGILAFVESAPGKPGSAQVLTGTGGDKGNLFQAAAAVTQALGNVAGKVNLAIQGQKLSSLVNDDGKGSVGRDLFQAAAQTTQVLSALIDTVG

SEQ ID NO: 16 - protein, 257 aa, Chlamydia trachomatis
MCIKRKKTWIAFLAVVCSFCLTGCLKEGGDSNSEKFIVGTNATYPPFEFVDKRGEVVGFDIDLAREISNKLGKTLDVREFSFDALILNLKQHRIDAVITGMSITPSRLKEILMIPYYGEEIKHLVLVFKGENKHPLPLTQYRSVAVQTGTYQEAYLQSLSEVHIRSFDSTLEVLMEVMHGKSPVAVLEPSIAQVVLKDFPALSTATIDLPEDQWVLGYGIGVASDRPALALKIEAAVQEIRKEGVLAELEQKWGLNN

SEQ ID NO: 17 - protein, 421 aa, Chlamydia trachomatis
MTASGGAGGLGSTQTVDVARAQAAAATQDAQEVIGSQEASEASMLKGCEDLINPAAATRIKKKGEKFESLEARRKPTADKAEKKSESTEEKGDTPLEDRFTEDLSEVSGEDFRGLKNSFDDDSSPDEILDALTSKFSDPTIKDLALDYLIQTAPSDGKLKSTLIQAKHQLMSQNPQAIVGGRNVLLASETFASRANTSPSSLRSLYFQVTSSPSNCANLHQMLASYLPSEKTAVMEFLVNGMVADLKSEGPSIPPAKLQVYMTELSNLQALHSVNSFFDRNIGNLENSLKHEGHAPIPSLTTGNLTKTFLQLVEDKFPSSSKAQKALNELVGPDTGPQTEVLNLFFRALNGCSPRIFSGAEKKQQLASVITNTLDAINADNEDYPKPGDFPRSSFSSTPPHAPVPQSEIPTSPTSTQPPSP

SEQ ID NO: 18 - protein, 221 aa, Chlamydia trachomatis
MKKFIYKYSFGALLLLSGLSGLSSCCANSYGSTLAKNTAEIKEESVTLREKPDAGCKKKSSCYLRKFFSRKKPKEKTEPVLPNFKSYADPMTDSERKDLSFVVSAAADKSSIALAMAQGEIKGALSRIREIHPLALLQALAEDPALIAGMKKMQGRDWVWNIFITELSKVFSQAASLGAFSVADVAAFASTLGLDSGTVTSIVDGERWAELIDVVIQNPAI

SEQ ID NO: 19 - protein, 242 aa, Chlamydia trachomatis
MKVKINDQFICISPYISARWNQIAFIESCDGGTEGGITLKLHLIDGETVSIPNLGQAIVDEVFQEHLLYLESTAPQKNKEEEKISSLLGAVQQMAKGCEVQVFSQKGLVSMLLGGAGSINVLLQHSPEHKDHPDLPTDLLERIAQMMRSLSIGPTSILAKPEPHCNCLHCQIGRATVEEEDAGVSDEDLTFRSWDISQSGEKMYTVTDPLNPEEQFNVYLGTPIGCTCGQPYCEHVKAVLYT

SEQ ID NO: 20 - protein, 448 aa, Chlamydia trachomatis
MLINFTFRNCLLFLVTLSSVPVFSAPQPRGTLPSSTTKIGSEVWIEQKVRQYPELLWLVEPSSTGASLKSPSGAIFSPTLFQKKVPAFDIAVRSLIHLHLLIQGSRQAYAQLIQLQTSESPLTFKQFLALHKQLTLFLNSPKEFYDSVKVLETAIVLRHLGCSTKAVAAFKPYFSEMQREAFYTKALHVLHTFPELSPSFARLSPEQKTLFFSLRKLANYDELLSLTNTPSFQLLSAGRSQRALLALDLYLYALDSCGEQGMSSQFHTNFAPLQSMLQQYATVEEAFSRYFTYRANRLGFDGSSRSEMALVRMATLMNLSPSEAAILTTSFKTLPTEEADTLINSFYTNKGDSLALSLRGLPTLVSELTRTAHGNTNAEARSQQIYATTLSLVVKSLKAHKEMLNKQILSKEIVLDFSETAASCQGLDIFSENVAVQIHLNGTVSIHL

SEQ ID NO: 21 - protein, 183 aa, Escherichia coli
CQGGQGGNGGGQQQKFMALGSGVIIDADKGYVVTNNHVVDNATVIKVQLSDGRKFDAKMVGKDPRSDIALIQIQNPKNLTAIKMADSDALRVGDYTVAIGNPFGLGETVTSGIVSALGRSGLNAENYENFIQTDAAINRGNSGGALVNLNGELIGINTAILAPDGGNIGIGFAIPSNMVKNLT

SEQ ID NO: 22 - protein, 179 aa, Bordetella bronchiseptica
GQVPGLSRREASTSLGSGVIVSAEGYVLTNYHVVEAADAIEVALADGRQAAAKVVGADPETDLAVLKLAGKLGELPVATFADKRAPRVGDVVLAIGNPFGVGQTTTQGIVSALGRNGLGINTYENFIQTDAAINPGNSGGALIDAHGDLVGINTAIYSETGGSLGIGFAIPVDSARRVM

SEQ ID NO: 23 - protein, 176 aa, Yersinia pestis
ATQQGLAIRTLGSGVIMSDKGYILTNKHVINDAEQIIVAMQNGRISEALLVGSDNLTDLAVLKIDATNLPVIPININRTPHIGDVVLAIGNPYNLGQTVTQGIISATGRIGLSSSGRQNFLQTDASINQGNSGGALVNTLGELMGINTLSFDKSNNGETPEGIGFAIPTALATKVM

SEQ ID NO: 24 - protein, 176 aa, Chlamydia muridarum
HREQPRPQQRDAVRGTGFIVSEDGYVVTNHHVVEDAGKIHVTLHDGQKYTAKIIGLDPKTDLAVIKIQAKNLPFLTFGNSDQLQIGDWSIAIGNPFGLQATVTVGVISAKGRNQLHIVDFEDFIQTDAAINPGNSGGPLLNIDGQVIGVNTAIVSGSGGYIGIGFAIPSLMAKRVI

SEQ ID NO: 25 - protein, 176 aa, Chlamydia trachomatis
HREQQRPQQRDAVRGTGFIVSEDGYVVTNHHVVEDAGKIHVTLHDGQKYTAKIVGLDPKTDLAVIKIQAEKLPFLTFGNSDQLQIGDWAIAIGNPFGLQATVTVGVISAKGRNQLHIVDFEDFIQTDAAINPGNSGGPLLNINGQVIGVNTAIVSGSGGYIGIGFAIPSLMAKRVI

SEQ ID NO: 26 - protein, 174 aa, Chlamydophila abortus
QKERPMSKEAVRGTGFIVSPDGYVVTNNHVVEDAGKIHVTLHDGQKYPAKVIGLDPKTDLAVIKINAEKLPHLTFGNSDNLKVGDWAIAIGNPFGLQATVTVGVISAKGRNQLHIADFEDFIQTDAAINPGNSGGPLLNIDGKVIGVNTAIVSGSGGYIGIGFAIPSLMAKKII

SEQ ID NO: 27 - protein, 174 aa, Chlamydophila pneumoniae
QREKPQSKEAVRGTGFLVSPDGYIVTNNHVVEDTGKIHVTLHDGQKYPATVIGLDPKTDLAVIKIKSQNLPYLSFGNSDHLKVGDWAIAIGNPFGLQATVTVGVISAKGRNQLHIADFEDFIQTDAAINPGNSGGPLLNIDGQVIGVNTAIVSGSGGYIGIGFAIPSLMANRII

SEQ ID NO: 28 - protein, 176 aa, Pseudomonas aeruginosa
VPRNPRGQQREAQSLGSGFIISNDGYILTNNHVVADADEILVRLSDRSEHKAKLIGADPRSDVAVLKIEAKNLPTLKLGDSNKLKVGEWVLAIGSPFGFDHSVTAGIVSAKGRSLPNESYVPFIQTDVAINPGNSGGPLLNLQGEVVGINSQIFTRSGGFMGLSFAIPIDVALNVA

SEQ ID NO: 29 - protein, 180 aa, Rickettsia conorii
NLEEVDQTPKSVPLGSGFIIEPNGLIVTNYHVIANVDKINIKLADNTELSAKLIGNDTKTDLALLKIDSEEPLPFVEFGDSNDARVGDWVIAIGNPFGNLGGTVTSGIISSKGRDIDIDTDNIVDNFIQTDAAINNGNSGGPMFNLDQKVIGVNTAIFSPLGTNIGIGFAIPSNTAKPII

SEQ ID NO: 30 - protein, 178 aa, Campylobacter jejuni
SQRKGKNDKEVVSSLGSGVIISKDGYIVTNNHVVDDADTITVNLPGSDIEYKAKLIGKDPKTDLAVIKIEANNLSAITFTNSDDLMEGDVVFALGNPFGVGFSVTSGIISALNKDNIGLNQYENFIQTDASINPGNSGGALVDSRGYLVGINSAILSRGGGNNGIGFAIPSNMVKDIA

SEQ ID NO: 31 - protein, 176 aa, Helicobacter pylori
GGMIPKERMERALGSGVIISKDGYIVTNNHVIDGADKIKVTIPGSNKEYSATLVGTDSESDLAVIRITKDNLPTIKFSDSNDISVGDLVFAIGNPFGVGESVTQGIVSALNKSGIGINSYENFIQTDASINPGNSGGALIDSRGGLVGINTAIISKTGGNHGIGFAIPSNMVKDTV

SEQ ID NO: 32 - protein, 183 aa, Yersinia pestis
GDLGGLGQGMPSKREFRSLGSGVIIDAGKGYVVTNNHVVDNANKISVKLSDGRSFDAKVIGKDPRTDIALLQLKDAKNLTAIKIANSDQLRVGDYTVAIGNPYGLGETVTSGIVSALGRSGLNVENYENFIQTDAAINRGNSGGALINLNGELIGINTAILAPDGGNIGIGFAIPSNMVKNLT

SEQ ID NO: 33 - protein, 175 aa, Vibrio parahaemolyticus
EQTRERPFRGLGSGVIIDAQKGHIVTNYHVIKGADEIRVRLYDGREYDAELVGGDEMADVALLKLEKAKNLTQIKIADSDKLRVGDFTVAIGNPFGLGQTVTSGIVSALGRSGLNVENFENFIQTDAAINSGNSGGALVNLNGELIGINTAILGPNGGNVGIGFAIPSNMMRNLT

SEQ ID NO: 34 - protein, 180 aa, Haemophilus influenzae
QFGGRGESKRNFRGLGSGVIINASKGYVLTNNHVIDGADKITVQLQDGREFKAKLVGKDEQSDIALVQLEKPSNLTEIKFADSDKLRVGDFTVAIGNPFGLGQTVTSGIVSALGRSTGSDSGTYENYIQTDAAVNRGNSGGALVNLNGELIGINTAIISPSGGNAGIAFAIPSNQASNLV

SEQ ID NO: 35 - protein, 200 aa, Streptococcus pyogenes
FGEGRSKENKDAELSIFSEGSGVIYRKDGNSAYVVTNNHVIDGAKRIEILMADGSKVVGELVGADTYSDLAVVKISSDKIKTVAEFADSTKLNVGEVAIAIGSPLGTQYANSVTQGIVSSLSRTVTLKNENGETVSTNAIQTDAAINPGNSGGPLINIEGQVIGINSSKISSTPTGSNGNSGAVEGIGFAIPSTDVIKII

SEQ ID NO: 36 - protein, 190 aa, Streptococcus pneumoniae
GNDDTDTDSQRISSEGSGVIYKKNDKEAYIVTNNHVINGASKVDIRLSDGTKVPGEIVGADTFSDIAVVKISSEKVTTVAEFGDSSKLTVGETAIAIGSPLGSEYANTVTQGIVSSLNRNVSLKSEDGQAISTKAIQTDTAINPGNSGGPLINIQGQVIGITSSKIATNGGTSVEGLGFAIPANDAINII

SEQ ID NO: 37 - protein, 185 aa, Listeria monocytogenes
GTTTSEQEASSGSGVIYKKANGKAYIVTNNHVVADANKLEVTFTNGKKSEAKLLGTDEWNDLAVLEIDDKNVTTVAAFGDSDSLKLGEPAIAIGSPLGTEFSGSVTQGIISGLNRAVPVDTNGDGTEDWEADVIQTDAAINPGNSGGALINIEGQVIGINSMKISMENVEGISFAIPSNTVEPII

SEQ ID NO: 38 - DNA, 1347 bp, Chlamydia trachomatis
atgttaataaactttacctttcgcaactgtcttttgttccttgtcacactgtctagtgtccctgttttctcagcacctcaacctcgcggaacgcttcctagctcgaccacaaaaattggatcagaagtttggattgaacaaaaagtccgccaatatccagagcttttatggttagtagagccgtcctctacgggagcctctttaaaatctccttcaggagccatcttttctccaacattattccaaaaaaaggtccctgctttcgatatcgcagtgcgcagtttgattcacttacatttattaatccagggttcccgccaagcctatgctcaactgatccaactacagaccagcgaatcccctctaacatttaagcaattccttgcattgcataagcaattaactctatttttaaattcccctaaggaattttatgactctgttaaagtgttagagacagctatcgtcttacgtcacttaggctgttcaactaaggctgttgctgcgtttaaaccttatttctcagaaatgcaaagagaggctttttacactaaggctctgcatgtactacacaccttcccagagctaagcccatcatttgctcgcctctctccggagcagaaaactctcttcttctccttgagaaaattggcgaattacgatgagttactctcgctgacgaacaccccaagttttcagcttctgtctgctgggcgctcgcaacgagctcttttagctctggacttgtacctctatgctttggattcctgtggagaacaggggatgtcctctcaattccacacaaacttcgcacctctacagtccatgttgcaacaatacgctactgtagaagaggccttttctcgttattttacttaccgagctaatcgattaggatttgatggctcttctcgatccgagatggctttagtaagaatggccaccttgatgaacttgtctccttccgaagctgcgattttaaccacaagcttcaaaacccttcctacagaagaagcggatactttgatcaatagtttctataccaataagggcgattcgttggctctttctctgcgagggttgcctacacttgtatccgaactgacgcgaactgcccatggcaataccaatgcagaagctcgatctcagcaaatttatgcaactaccctatcgctagtagtaaagagtctgaaagcgcacaaagaaatgctaaacaagcaaattctttctaaggaaattgttttagatttctcagaaactgcagcttcttgccaaggattggatatcttttccgagaatgtcgctgttcaaattcacttaaatggaaccgttagtatccatttataa

SEQ ID NO: 39 - DNA, 2433 bp, Chlamydia trachomatis
atgactaagccttctttcttatacgttattcaacctttttccgtatttaatccacgattaggacgtttctctacagactcagatacttatatcgaagaagaaaaccgcctagcatcgttcattgagagtttgccactggagatcttcgatataccttctttcatggaaaccgcgatttccaatagcccctatattttatcttgggagacaactaaagacggcgctctgttcactattcttgaacccaaactctcagcttgcgcagccacttgcctggtagccccttctatacaaatgaaatccgatgcggagctcctagaagaaattaagcaagcgttattacgcagctctcatgacggtgtgaaatatcgcatcaccagagaatccttctctccagaaaagaaaactcctaaggttgctctagtcgatgacgatattgaattgattcgcaatgtcgactttttgggtagagctgttgacattgtcaaattagaccctattaatattctgaataccgtaagcgaagagaatattctagattactcttttacaagagaaacggctcagctgagcgcggatggtcgttttggtattcctccagggactaagctattccctaaaccttcttttgatgtagaaatcagtacctccattttcgaagaaacaacttcatttactcgaagtttttctgcatcggttacttttagtgtaccagacctcgcggcgactatgcctcttcaaagccctcccatggtagaaaatggtcaaaaagaaatttgtgtcattcaaaaacacttattcccaagctactctcctaaactagtcgatattgttaaacgatacaaaagagaggctaagatcttgattaacaagcttgcctttggaatgttatggcgacatcgggctaaaagccaaatcctcaccgagggaagcgtacgtctagacttacaaggattcacagaatcgaagtacaattaccagattcaagtaggatcccatacgattgcagctgtattaatcgatatggatatttccaagattcaatccaaatcagaacaagcttatgcaattaggaaaatcaaatcaggctttcaacgtagcttggatgactatcatatttatcaaattgaaagaaaacaaaccttttctttttctccgaagcatcgcagcctctcatccacatcccattccgaagattctgatttggatctttctgaagcagccgccttttcaggaagtcttacctgcgagtttgtaaaaaaaagcactcaacatgccaagaataccgtcacatgttccacagccgctcattccctatacacactcaaagaagatgacagctcgaacccctctgaaaaacgattagatagttgtttccgcaattggattgaaaacaaactaagcgccaattctccagattcctggtcagcgtttattcaaaaattcggaacacactatattgcatcagcaacttttggagggataggtttccaagtgctcaaactatcttttgaacaggtggaggatctacatagcaaaaagatctccttagaaaccgcagcagccaactctctattaaaaggttctgtatccagcagcacagaatctggatactccagctatagctccacgtcttcttctcatacggtatttttaggaggaacggtcttaccttcggttcatgatgaacgtttagactttaaagattggtcggaaagtgtgcacctggaacctgttcctatccaggtttctttacaacctataacgaatttactagttcctctccattttcctaatatcggtgctgcagagctctctaataaacgagaatctcttcaacaagcgattcgagtctatctcaaagaacataaagtagatgagcaaggagaacgtactacatttacatcaggaatcgataatccttcttcctggtttaccttagaagctgcccactctcctcttatagtcagtactccttacattgcttcgtggtctacgcttccttatttgttcccaacattaagagaacgttcttcggcaacccctatcgttttctatttttgtgtagataataatgaacatgcttcgcaaaaaatattaaaccaatcgtattgcttcctcgggtccttgcctattcgacaaaaaatttttggtagcgaatttgctagtttcccctatctatctttctatggaaatgcaaaagaggcgtactttgataacacgtactacccaacgcgttgtgggtggattgttgaaaagttaaatactacacaagatcaattcctccgggatggagacgaggtgcgactaaaacatgtttccagcggaaagtatctagcaacaactcctcttaaggatacccatggtacactcacgcgtacaacgaactgtgaagatgctatctttattattaaaaaatcttcaggttattga

SEQ ID NO: 40 - DNA, 603 bp, Chlamydia trachomatis
atgctcgctaatcgcttattcttaataacccttttagggttaagttcgtctgtttacggcgcaggtaaagcaccgtctttgcaggctattctagccgaagtcgaagacacctcctctcgtctacacgctcatcacaatgagcttgctatgatctctgaacgcctcgatgagcaagacacgaaactacagcaactttcgtcaacacaagatcataacctacctcgacaagttcagcgactagaaacggaccaaaaagctttggcaaaaacactggcgattctttcgcaatccgtccaagatattcggtcttctgtacaaaataaattacaagaaatccaacaagaacaaaaaaaattagcacaaaatttgcgagcgcttcgtaactctttacaagctctcgttgatggctcttctccagaaaattatattgatttcctaactggtgaaaccccggaacatattcatattgttaaacaaggagagaccctgagcaagatcgcgagtaaatataacatccccgtcgtagaattaaaaaaacttaataaactaaattcggatactatttttacagatcaaagaattcgccttccgaaaaagaaatag

SEQ ID NO: 41 - DNA, 951 bp, Chlamydia trachomatis
atggcatccaagtctcgccattatcttaatcagccttggtacattatcttattcatctttgttcttagtttaattgctggtaccctcctgtcttctgtgtattatgtccttgcacctatccaacagcaagctgcggaattcgatcgcaatcaacaaatgctaatggctgcacaagtaatttcttccgataacacattccaagtctatgaaaagggagattggcacccagccctatataatactaaaaagcagttgctagagatctcctctactcctcctaaagtaaccgtgacaactttaagctcatattttcaaaactttgttagagtcttgcttacagatacacaaggaaatctttcttcattcgaagaccataatctcaatctagaagaatttttatctcaaccaactcctgtaatacatggtcttgccctttatgtggtctacgctatcctacacaacgatgcagcttcctctaaattatctgcttcccaagtagcgaaaaatccaacagctatagaatctatagttcttcctatagaaggttttggtttgtggggacctatctatggattccttgctctagaaaaagacgggaatactgttcttggtacttcttggtatcaacatggcgagactcctggattaggagcaaatatcgctaaccctcaatggcaaaaaaatttcagaggcaaaaaagtatttctagtctcagcttctggagaaacagattttgctaagacaaccctaggactggaagttataaaaggatctgtatctgcagcattaggagactcacctaaagctgcttcttccatcgacggaatttcaggagctactttgacttgtaatggtgttaccgaatccttctctcattctctagctccctaccgcgctttgttgactttcttcgccaactctaaacctagtggagagtctcatgaccactaa

SEQ ID NO: 42 - DNA, 1662 bp, Chlamydia trachomatis
atgcgaataggagatcctatgaacaaactcatcagacgagcagtgacgatcttcgcggtgactagtgtggcgagtttatttgctagcggggtgttagagacctctatggcagagtctctctctacaaacgttattagcttagctgacaccaaagcgaaagacaacacttctcataaaagcaaaaaagcaagaaaaaaccacagcaaagagactcccgtagaccgtaaagaggttgctccggttcatgagtctaaagctacaggacctaaacaggattcttgctttggcagaatgtatacagtcaaagttaatgatgatcgcaatgttgaaatcacacaagctgttcctgaatatgctacggtaggatctccctatcctattgaaattactgctacaggtaaaagggattgtgttgatgttatcattactcagcaattaccatgtgaagcagagttcgtacgcagtgatccagcgacaactcctactgctgatggtaagctagtttggaaaattgaccgcttaggacaaggcgaaaagagtaaaattactgtatgggtaaaacctcttaaagaaggttgctgctttacagctgcaacagtatgcgcttgtccagagatccgttcggttacaaaatgtggacaacctgctatctgtgttaaacaagaaggcccagagaatgcttgtttgcgttgcccagtagtttacaaaattaatatagtgaaccaaggaacagcaacagctcgtaacgttgttgttgaaaatcctgttccagatggttacgctcattcttctggacagcgtgtactgacgtttactcttggagatatgcaacctggagagcacagaacaattactgtagagttttgtccgcttaaacgtggtcgtgctaccaatatagcaacggtttcttactgtggaggacataaaaatacagcaagcgtaacaactgtgatcaacgagccttgcgtacaagtaagtattgcaggagcagattggtcttatgtttgtaagcctgtagaatatgtgatctccgtttccaatcctggagatcttgtgttgcgagatgtcgtcgttgaagacactctttctcccggagtcacagttcttgaagctgcaggagctcaaatttcttgtaataaagtagtttggactgtgaaagaactgaatcctggagagtctctacagtataaagttctagtaagagcacaaactcctggacaattcacaaataatgttgttgtgaagagctgctctgactgtggtacttgtacttcttgcgcagaagcgacaacttactggaaaggagttgctgctactcatatgtgcgtagtagatacttgtgaccctgtttgtgtaggagaaaatactgtttaccgtatttgtgtcaccaacagaggttctgcagaagatacaaatgtttctttaatgcttaaattctctaaagaactgcaacctgtatccttctctggaccaactaaaggaacgattacaggcaatacagtagtattcgattcgttacctagattaggttctaaagaaactgtagagttttctgtaacattgaaagcagtatcagctggagatgctcgtggggaagcgattctttcttccgatacattgactgttccagtttctgatacagagaatacacacatctattaa

SEQ ID NO: 43 - DNA, 1329 bp, Chlamydia trachomatis
atgcaggctgcacaccatcactatcaccgctacacagataaactgcacagacaaaaccataaaaaagatctcatctctcccaaacctaccgaacaagaggcgtgcaatacttcttcccttagtaaggaattaatccctctatcagaacaaagaggccttttatcccccatctgtgactttatttcggaacgcccttgcttacacggagtttctgttagaaatctcaagcaagcgctaaaaaattctgcaggaacccaaattgcactggattggtctattctccctcaatggttcaatcctcgggtctctcatgcccctaagctttctatccgagactttgggtatagcgcacaccaaactgttaccgaagccactcctccttgctggcaaaactgctttaatccatctgcggccgttactatctatgattcctcatatgggaaaggggtctttcaaatatcctatacccttgtccgctattggagagagaatgctgcgactgctggcgatgctatgatgctcgcagggagtatcaatgattatccctctcgtcagaacattttctctcagtttactttctcccaaaacttcccaaatgaacgggtgagtctgacaattggtcagtactcactctatgcaatagacggaacattatacaataacgatcaacaacttggattcattagttacgcattatcacaaaatccaacagcaacttattcctctggaagtcttggagcttacctacaagtcgctcctaccgcaagcacaagtcttcaaataggatttcaagacgcttataatatctccggatcctctatcaaatggagtaaccttacaaaaaatagatacaattttcacggttttgcttcctgggctccccgctgttgcttaggatctggccagtactccgtgcttctttatgtgactagacaagttccagaacagatggaacaaacaatgggatggtcagtcaatgcgagtcaacacatatcttctaaactgtatgtgtttggaagatacagcggtgttacaggacatgtgttcccgattaaccgcacgtattcattcggtatggcctctgcaaatttatttaaccgtaacccacaagatttatttggaattgcttgcgcattcaataatgtacacctctctgcttctccaaatactaaaagaaaatacgaaactgtaatcgaagggtttgcaactatcggttgcggcccctatctttctttcgctccagacttccaactctacctctacccagctcttcgtccaaacaaacaatctgcccgtgtttatagcgtgcgagctaatttagctatctaa

SEQ ID NO: 44 - DNA, 3018 bp, Chlamydia trachomatis
atgacgaattctatatcaggttatcaacctactgttacaacttctacatcatcaaccacttcggcatcaggtgcttccggatctctgggagcttcttctgtatctactaccgcaaacgctacagttacacaaacagcaaacgcaacaaattcagcggctacatcttctatccaaacgactggagagactgtagtaaactatacgaattcagcctccgcccccaatgtaactgtatcgacctcctcttcttccacacaagccacagccacttcgaataaaacttcccaagccgttgctggaaaaatcacttctccagatacttcagaaagctcagaaactagctctacctcatcaagcgatcatatccctagcgattacgatgacgttggtagcaatagtggagatattagcaacaactacgatgacgtaggtagtaacaacggagatatcagtagcaattatgacgatgctgctgctgattacgagccgataagaactactgaaaatatttatgagagtattggtggctctagaacaagtggcccagaaaatacaagtggtggtgcagcagcagcactcaattctctaagaggctcctcctacagcaattatgacgatgctgctgctgattacgagccgataagaactactgaaaatatttatgagagtattggtggctctagaacaagtggcccagaaaatacgagtggtggtgcagcagcagcactcaattctctaagaggctcctcctacagcaattatgacgatgctgctgctgattacgagccgataagaactactgaaaatatttatgagagtattggtggctctagaacaagtggcccagaaaatacgagtgatggtgcagcagcagcagcactcaattctctaagaggctcctcctacacaacagggcctcgtaacgagggtgtattcggccctggaccggaaggactaccagacatgtctcttccttcatacgatcctacaaataaaacctcgttattgactttcctctccaaccctcatgtaaagtcgaaaatgcttgaaaactcggggcatttcgtcttcattgatacagatagaagtagtttcattcttgttcctaacggaaattgggaccaagtctgttcaattaaagttcaaaatggaaagaccaaagaagatctcgacatcaaagacttggaaaacatgtgtgcaaaattctgtacagggtttagcaaattctctggtgactgggacagtcttgtagaacctatggtgtcagccaaagctggagtggccagcggaggcaatcttcccaatacagtgattatcaataataaattcaaaacttgcgttgcttatggtccttggaatagccaggaagcaagttctggttatacaccttctgcttggagacgtggtcatcgagtagattttggaggaatttttgagaaagccaacgactttaataaaatcaactggggaactcaagccgggcctagtagcgaagacgatggcatttccttctccaatgaaactcctggagctggtcctgcagctgctccatcaccaacgccatcctctattcctatcatcaatgtcaatgtcaatgttggcggaactaatgtgaatattggagatacgaatgtcaacacgactaacaccacaccaacaactcaatctacagacgcctctacagatacaagcgatatcgatgacataaataccaacaaccaaactgatgatatcaatacgacagacaaagactctgacggagctggtggagtcaatggcgatatatccgaaacagaatcctcttctggagatgattcaggaagtgtctcttcctcagaatcagacaagaatgcctctgtcggaaatgacggacctgctatgaaagatatcctttctgccgtgcgtaaacacctagacgtcgtttaccctggcgaaaatggcggttctacagaagggcctctcccagctaaccaaactctcggagacgtaatctctgatgtagagaataaaggctccgctcaggatacaaaattgtcaggaaatacaggagctggggatgacgatccaacaaccacagctgctgtaggtaatggagcggaagagatcactctttccgacacagattctggtatcggagatgatgtatccgatacagcgtcttcatctggggatgaatccggaggagtctcctctccctcttcagaatccaataaaaatactgccgttggaaatgacggaccttctggactagatatcctcgctgccgtacgtaaacatttagataaggtttaccctggcgacaatggtggttctacagaagggcctctccaagctaaccaaactcttggagatatcgtccaggatatggaaacaacagggacatcccaagaaaccgttgtatccccatggaaaggaagcacttcttcaacggaatcagcaggaggaagtggtagcgtacaaacactactgccttcaccacctccaaccccgtcaactacaacattaagaacgggcacaggagctaccaccacatccttgatgatgggaggaccaatcaaagctgacataataacaactggtggcggaggacgaattcctggaggaggaacgttagaaaagctgctccctcgtatacgtgcgcacttagacatatcctttgatgcgcaaggcgatctcgtaagtactgaagagcctcagcttggctcgattgtaaacaaattccgccaagaaactggttcaagaggaatcttagctttcgttgagagtgctccaggcaagccgggatctgcacaggtcttaacgggtacagggggagataaaggcaacctattccaagcagctgccgcagtcacccaagccttaggaaatgttgcagggaaagtcaaccttgcgatacaaggccaaaaactatcatccctagtcaatgacgacgggaaggggtctgttggaagagatttattccaagcagcagcccaaacaactcaagtgctaagcgcactgattgataccgtaggataa

SEQ ID NO: 45 - DNA, 774 bp, Chlamydia trachomatis
atgtgcataaaaagaaaaaaaacatggatagcttttttagcagttgtctgtagtttttgtttgacgggttgtttaaaagaagggggagactccaatagtgaaaaatttattgtagggactaatgcaacctaccctccttttgagtttgttgataagcgaggagaggttgtaggcttcgatatagacttggctagagagattagtaacaagctggggaaaacgctggacgttcgggagttttcctttgatgcactcattctaaacctaaaacagcatcggattgatgcggttataacagggatgtccattactccttctagattgaaggaaattcttatgattccctattatggggaggaaataaaacacttggttttagtgtttaaaggagagaataagcatccattgccactcactcaatatcgttctgtagctgttcaaacaggaacctatcaagaggcctatttacagtctctttctgaagttcatattcgctcttttgatagcactctagaagtactcatggaagtcatgcatggtaaatctcccgtcgctgttttagagccatctatcgctcaagttgtcttgaaagatttcccggctctttctacagcaaccatagatctccctgaagatcagtgggttttaggatacgggattggcgttgcttcagatcgcccagctttagccttgaaaatcgaggcagctgtgcaagagatccgaaaagaaggagtgctagcagagttggaacagaagtggggtttgaacaactaa

SEQ ID NO: 46 - DNA, 504 bp, Chlamydia trachomatis
atgtccaggcagaatgctgaggaaaatctaaaaaattttgctaaagagcttaaactccccgacgtggccttcgatcagaataatacgtgcattttgtttgttgatggagagttttctcttcacctgacctacgaagaacactctgatcgcctttatgtttacgcacctcttcttgacggactgccagacaatccgcaaagaaggttagctctatatgagaagttgttagaaggctctatgctcggaggccaaatggctggtggaggggtaggagtcgctactaaggaacagttgatcttaatgcactgcgtgttagacatgaagtatgcagagaccaacctactcaaagcttttgcacagctttttattgaaaccgttgtgaaatggcgaactgtttgttctgatatcagcgctggacgagaacccactgttgataccatgccacaaatgcctcaagggggtggcggaggaattcaacctcctccagcaggaatccgtgcataa

SEQ ID NO: 47 - DNA, 957 bp, Chlamydia muridarum
atggcatccaagtctcgtcattatcttaaccagccttggtacattatcttattcatctttgttcttagtctggttgctggtacccttctttcttcagtttcctatgttctatctccaatccaaaaacaagctgcagaatttgatcgtaatcagcaaatgttgatggccgcacaaattatttcctatgacaataaattccaaatatatgctgaaggggattggcaacctgctgtctataatacaaaaaaacagatactagaaaaaagctcttccactccaccacaagtgactgtggcgactctatgctcttattttcaaaattttgttagagttttgcttacagactcccaagggaatctttcttcttttgaagatcacaatcttaacctagaagagttcttatcccaccccacatcttcagtacaagatcactctctgcatgtaatttatgctattctagcaaacgatgaatcctctaaaaagttatcatcctcccaagtagcaaaaaatccggtatccatagagtctattattcttcctataaaaggatttggtttatggggaccaatctatggatttcttgctttagaaaaggacggtaatacggttctagggacatgctggtatcaacatggtgagactccaggattaggagcaaatataactaatccccaatggcaacaaaatttcagaggaaaaaaagtatttctcgcttcctcttccggagaaaccgattttgctaaaacaactctaggactagaagttataaaaggatctgtttctgcattattaggggactctcccaaagctaattccgctgttgatggaatttcaggagctacactgacctgtaatggagttactgaagcttttgctaattcgctagctccttaccgccccttattgactttcttcgccaatcttaactctagtggagaatctcatgacaaccaataa

SEQ ID NO: 48 - protein, 318 aa, Chlamydia muridarum
MASKSRHYLNQPWYIILFIFVLSLVAGTLLSSVSYVLSPIQKQAAEFDRNQQMLMAAQIISYDNKFQIYAEGDWQPAVYNTKKQILEKSSSTPPQVTVATLCSYFQNFVRVLLTDSQGNLSSFEDHNLNLEEFLSHPTSSVQDHSLHVIYAILANDESSKKLSSSQVAKNPVSIESIILPIKGFGLWGPIYGFLALEKDGNTVLGTCWYQHGETPGLGANITNPQWQQNFRGKKVFLASSSGETDFAKTTLGLEVIKGSVSALLGDSPKANSAVDGISGATLTCNGVTEAFANSLAPYRPLLTFFANLNSSGESHDNQ

SEQ ID NO: 49 - DNA, 1416 bp, Chlamydia muridarum
atgaatggaaaagttctgtgtgaggtttctgtgtccttccgttcgattctgctgacggctctgctttcactttcttttacaaacactatgcaggctgcacaccatcattatcaccgttatgatgataaactacgcagacaataccataaaaaggacttgcccactcaagagaatgttcggaaagagttttgtaatccctactctcatagtagtgatcctatccctttgtcacaacaacgaggagtcctatctcctatctgtgatttagtctcagagtgctcgtttttgaacgggatttccgttaggagtcttaaacaaacactgaaaaattctgctgggactcaagttgctttagactggtctatccttcctcaatggttcaatcctagatcctcttgggctcctaagctctctattcgagatcttggatatggtaaaccccagtcccttattgaagcagattccccttgttgtcaaacctgcttcaacccatctgctgctattacgatttacgattcttcatgtgggaagggtgttgtccaagtgtcatacacccttgttcgttattggagagaaacggctgcacttgcagggcaaactatgatgcttgcaggaagtattaatgattatcctgctcgccaaaacatattctctcaacttacattttcccaaactttccctaatgagagagtaaatctaactgttggtcaatactctctttactcgatagacggaacgctgtacaacaatgatcagcagctaggatttattagttatgcgttgtcgcaaaatccaacagcgacttattcctctggaagccttggcgcctatctacaagtcgctccaacagaaagcacctgtcttcaagttgggttccaagatgcctataatatttcaggttcctcgatcaaatggaataatcttacaaaaaataagtataacttccatggctatgcatcttgggctccacactgttgcttaggacctggacaatactctgttcttctttatgtaaccagaaaggttcctgagcaaatgatgcagacaatgggctggtctgtgaatgcaagtcaatacatctcttctaaactttatgtatttggaagatacagcggagtcacaggccaattgtctcctattaaccgaacctattcatttggcttagtctctcctaatttattgaaccgtaacccacaagacttatttggagtagcttgcgcattcaataatatacacgcctccgcctttcaaaatgctcaaagaaaatatgaaactgtgatcgagggatttgcaactattggttgcggaccttacatctcctttgctccagatttccaactttacctctatcctgctctgcgtccaaataaacaaagcgcccgagtctatagcgttcgcgcaaacctagctatttag

SEQ ID NO: 50 - protein, 471 aa, Chlamydia muridarum
MNGKVLCEVSVSFRSILLTALLSLSFTNTMQAAHHHYHRYDDKLRRQYHKKDLPTQENVRKEFCNPYSHSSDPIPLSQQRGVLSPICDLVSECSFLNGISVRSLKQTLKNSAGTQVALDWSILPQWFNPRSSWAPKLSIRDLGYGKPQSLIEADSPCCQTCFNPSAAITIYDSSCGKGVVQVSYTLVRYWRETAALAGQTMMLAGSINDYPARQNIFSQLTFSQTFPNERVNLTVGQYSLYSIDGTLYNNDQQLGFISYALSQNPTATYSSGSLGAYLQVAPTESTCLQVGFQDAYNISGSSIKWNNLTKNKYNFHGYASWAPHCCLGPGQYSVLLYVTRKVPEQMMQTMGWSVNASQYISSKLYVFGRYSGVTGQLSPINRTYSFGLVSPNLLNRNPQDLFGVACAFNNIHASAFQNAQRKYETVIEGFATIGCGPYISFAPDFQLYLYPALRPNKQSARVYSVRANLAI

SEQ ID NO: 51 - DNA, 1665 bp, Chlamydia muridarum
atgcgaataggagatcctatgaacaaactcatcagacgagctgtgacgatcttcgcggtgactagtgtggcgagtttatttgctagcggggtgttagagacctctatggcagagtctctctctaccaacgttattagcttagctgacaccaaagcgaaagagaccacttctcatcaaaaagacagaaaagcaagaaaaaatcatcaaaataggacttccgtagtccgtaaagaggttactgcagttcgtgatactaaagctgtagagcctagacaggattcttgctttggcaaaatgtatacagtcaaagttaatgatgatcgtaatgtagaaatcgtgcagtccgttcctgaatatgctacggtaggatctccatatcctattgagattactgctatagggaaaagagactgtgttgatgtaatcattacacagcaattaccatgcgaagcagagtttgttagcagtgatccagctactactcctactgctgatggtaagctagtttggaaaattgatcggttaggacagggcgaaaagagtaaaattactgtatgggtaaaacctcttaaagaaggttgctgctttacagctgcaacggtttgtgcttgtccagagatccgttcggttacgaaatgtggccagcctgctatctgtgttaaacaggaaggtccagaaagcgcatgtttgcgttgcccagtaacttatagaattaatgtagtcaaccaaggaacagcaacagcacgtaatgttgttgtggaaaatcctgttccagatggctatgctcatgcatccggacagcgtgtattgacatatactcttggggatatgcaacctggagaacagagaacaatcaccgtggagttttgtccgcttaaacgtggtcgagtcacaaatattgctacagtttcttactgtggtggacacaaaaatactgctagcgtaacaacagtgatcaatgagccttgcgtgcaagttaacatcgagggagcagattggtcttatgtttgtaagcctgtagaatatgttatctctgtttctaaccctggtgacttagttttacgagacgttgtaattgaagatacgctttctcctggaataactgttgttgaagcagctggagctcagatttcttgtaataaattggtttggactttgaaggaactcaatcctggagagtctttacaatataaggttctagtaagagctcaaactccagggcaattcacaaacaacgttgttgtgaaaagttgctctgattgcggtatttgtacttcttgcgcagaagcaacaacttactggaaaggagttgctgctactcatatgtgcgtagtagatacttgtgatcctatttgcgtaggagagaacactgtttatcgtatctgtgtgacaaacagaggttctgctgaagatacaaatgtgtccttaattttgaaattctctaaagaattacaacctatatctttctctggaccaactaaaggaaccattacaggaaacacggtagtgtttgattcgttacctagattaggttctaaagaaactgtagagttttctgtaacgttgaaagcagtatccgctggagatgctcgtggggaagctattctttcttccgatacattgacagttcctgtatctgatacggagaatacacatatctattaa

SEQ ID NO: 52 - protein, 554 aa, Chlamydia muridarum
MRIGDPMNKLIRRAVTIFAVTSVASLFASGVLETSMAESLSTNVISLADTKAKETTSHQKDRKARKNHQNRTSVVRKEVTAVRDTKAVEPRQDSCFGKMYTVKVNDDRNVEIVQSVPEYATVGSPYPIEITAIGKRDCVDVIITQQLPCEAEFVSSDPATTPTADGKLVWKIDRLGQGEKSKITVWVKPLKEGCCFTAATVCACPEIRSVTKCGQPAICVKQEGPESACLRCPVTYRINVVNQGTATARNVVVENPVPDGYAHASGQRVLTYTLGDMQPGEQRTITVEFCPLKRGRVTNIATVSYCGGHKNTASVTTVINEPCVQVNIEGADWSYVCKPVEYVISVSNPGDLVLRDVVIEDTLSPGITVVEAAGAQISCNKLVWTLKELNPGESLQYKVLVRAQTPGQFTNNVVVKSCSDCGICTSCAEATTYWKGVAATHMCVVDTCDPICVGENTVYRICVTNRGSAEDTNVSLILKFSKELQPISFSGPTKGTITGNTVVFDSLPRLGSKETVEFSVTLKAVSAGDARGEAILSSDTLTVPVSDTENTHIY

SEQ ID NO: 53 - DNA, 504 bp, Chlamydia muridarum
atgtccagacagaatgctgaggaaaatctaaaaaattttgctaaagagctcaagctccccgacgtggccttcgatcagaataatacgtgcattttgtttgttgatggagagttttctcttcacctgacctacgaagagcactctgatcgcctttatgtttacgcacctctccttgacggactcccagataatccgcaaagaaagttggctctgtatgagaaattgttggaaggctctatgctcggaggccaaatggctggtggaggagtaggagttgctactaaagaacagttgatcctaatgcattgcgtgttagatatgaaatatgcagagactaatctattgaaagcttttgcacagcttttcattgaaactgttgtgaaatggcgaacggtctgttctgatatcagcgctggacgagaaccttccgttgacactatgcctcaaatgcctcaaggaggcagcggaggaattcaacctcctccaacaggaattcgtgcgtag

SEQ ID NO: 54 - protein, 167 aa, Chlamydia muridarum
MSRQNAEENLKNFAKELKLPDVAFDQNNTCILFVDGEFSLHLTYEEHSDRLYVYAPLLDGLPDNPQRKLALYEKLLEGSMLGGQMAGGGVGVATKEQLILMHCVLDMKYAETNLLKAFAQLFIETVVKWRTVCSDISAGREPSVDTMPQMPQGGSGGIQPPPTGIRA

SEQ ID NO: 55 - DNA, 609 bp, Chlamydia muridarum
atgctcgctaatcggttatttctaatcacccttataggttttggctattctgcttacggtgccagcacagggaaatcaccttctttacaggttattttagctgaagtcgaggatacatcttcgcgcttacaagctcatcagaatgagcttgttatgctctcggaacgtttagatgagcaagacacaaaacttcaacaactctcgtcaactcaggcccgtaatcttcctcaacaagttcaacggcttgagattgatctgagagctctggctaaaacagctgctgtgctctcgcaatctgttcaggatatccgatcatccgtgcaaaataaattacaagaaatccaacaagaacaaaaaaatttagctcaaaatttacgagcgcttcgcaactccttacaagcactagttgatggctcttccccagaaaattatattgattttttggccggggagacacctgaacatattcacgttgttaaacaaggagaaaccctgagtaaaatcgctagtaagtacaatatccctgtcgcagaattgaaaaaacttaataaattaaattccgatactatttttactgatcaaagaatccgacttccaaaaaagaaataa

SEQ ID NO: 56 - protein, 202 aa, Chlamydia muridarum
MLANRLFLITLIGFGYSAYGASTGKSPSLQVILAEVEDTSSRLQAHQNELVMLSERLDEQDTKLQQLSSTQARNLPQQVQRLEIDLRALAKTAAVLSQSVQDIRSSVQNKLQEIQQEQKNLAQNLRALRNSLQALVDGSSPENYIDFLAGETPEHIHVVKQGETLSKIASKYNIPVAELKKLNKLNSDTIFTDQRIRLPKKK

SEQ ID NO: 57 - DNA, 3024 bp, Chlamydia muridarum
atgacgactccaataagtaattctccatcttctattccaactgttacagtatcaactactacagcatcttctggatctctcggaacttctactgtatcatcaacgactacaagtacttcagtcgcacaaacagcaacaacaacatcttctgcttctacatctataattcagtctagtggagaaaacatccaatccactacaggtaccccttctcctattacgtctagtgtttcaacatccgctccatctcctaaagcctccgccactgcaaacaaaacttcaagcgctgtttctgggaaaattacctcacaagaaacttctgaggaatccgaaacccaagccactacatctgatggagaagttagtagtaattacgatgatgttgataccccgaccaattcgtccgattcgacagttgatagtgattaccaagatgttgagactcagtacaaaacaattagcaacaatggtgaaaacacttatgaaacaatcggaagtcatggtgagaaaaacacacacgtccaggaaagccatgcatccggaacaggaaatcccataaataatcagcaagaagctattagacagctccgatcatctacctatacaaccagccctcgtaatgagaatatatttagtccaggaccggaaggtctacctaatatgtctcttcctagttacagccctacagataaaagttctctactagctttcctatctaatcccaatacaaaagcaaaaatgctcgaacactccgggcatttagtctttatagacacaactagaagtagctttatctttgttccgaatggaaattgggatcaagtctgttccatgaaggttcagaatgggaaaactaaagaagaccttggcttaaaggacttagaagatatgtgtgcaaagttttgcacaggatacaataaattctcctctgattggggaaatcgagttgaccccttggtctcttctaaggccgggatagaaagtggggggcacctcccaagctcagttatcatcaacaacaaatttagaacctgtgttgcctatgggccgtggaaccccaaagaaaacggccccaattatactccttcagcctggagacgtgggcatcgagtagattttggaaagatctttgatggaacagcgccgtttaataaaatcaactggggctcttcccctacccctggtgatgacggcatctccttctctaatgaaactattgggtctgaaccattcgcgacacctccctcatccccatcgcaaacccccgttatcaacgtcaatgttaatgtcggtggaaccaatgttaatattggggatacaaacgtatctaaaggatccggcacaccaacatcttctcaatctgtggacatgtctacagatactagcgatttagataccagtgatattgatacaaacaaccaaactaacggcgatatcaacacgaatgacaactccaataatgtcgatggaagtttatctgacgttgattcaagggtggaagacgatgacggtgtatcggatacagagtccactaatggcaatgactctggtaaaactacttccacagaagaaaatggtgacccaagcggaccagacatcctggctgctgtacgtaaacacctagacactgtctatccaggagaaaatggcggatctacagaaggacctctccctgctaatcaaaatctggggaacgttatccatgatgtggagcagaatggatctgctaaagaaactattatcactccaggagatacagggcctacagactcaagctcctctgtagatgctgatgcagacgttgaagatacttctgatactgactctggaatcggagacgacgacggtgtatcggatacagagtccactaatggtaataactctggtaaaactacttccacagaagaaaatggtgacccaagcggaccagacatcctggctgctgtacgtaaacacctagacactgtctatccaggagaaaatggcggatctacagaaggacctctccctgctaatcaaaatctggggaacgttatccatgatgtagaacaaaacggagccgctcaagaaactattatcactccaggagatacggaatctacagacacaagctctagtgtaaatgctaatgcagacttagaagatgtttctgatgctgattcaggattcggggatgatgacggtatatcggatacagagtccactaatggtaacgactctggaaaaaatactcctgtaggggatggtggtacaccaagcggaccagatatcctagctgctgtacgcaaacatctagacactgtctatccaggagaaaatggtggatctacagagagacctttacccgctaatcaaaatttaggagatatcattcatgatgtagaacaaaacggaagcgctaaagaaactgtagtatcgccttatcgaggaggaggaggaaatacatcttccccaattggattagcctccctgcttccagcaacaccatccacacctttgatgacaacacctagaacaaatgggaaagctgcagcttcttctttgatgataaaaggaggagaaactcaagccaagctagttaagaatggcggcaatatccctggagaaaccacattagcagaattactccctcgtttaagaggacaccttgacaaagtctttacttcagacgggaagtttacaaatcttaatggacctcaacttggagccatcatagaccaattccgcaaagaaacgggttccggaggaatcatagctcatacagatagtgttccaggagagaacggaacagcctctcctctcacaggaagttcaggggaaaaagtctctctctatgatgcagcgaaaaacgtcactcaagctttaacaagtgttacgaacaaagtaaccctagcaatgcaaggacaaaaactggaaggaattataaacaacaacaataccccctcttctattggacaaaatcttttcgcagcagcgagggcaacgacacaatccctcagttcattaattggaaccgtacaataa

SEQ ID NO: 58 - protein, 1007 aa, Chlamydia muridarum
MTTPISNSPSSIPTVTVSTTTASSGSLGTSTVSSTTTSTSVAQTATTTSSASTSIIQSSGENIQSTTGTPSPITSSVSTSAPSPKASATANKTSSAVSGKITSQETSEESETQATTSDGEVSSNYDDVDTPTNSSDSTVDSDYQDVETQYKTISNNGENTYETIGSHGEKNTHVQESHASGTGNPINNQQEAIRQLRSSTYTTSPRNENIFSPGPEGLPNMSLPSYSPTDKSSLLAFLSNPNTKAKMLEHSGHLVFIDTTRSSFIFVPNGNWDQVCSMKVQNGKTKEDLGLKDLEDMCAKFCTGYNKFSSDWGNRVDPLVSSKAGIESGGHLPSSVIINNKFRTCVAYGPWNPKENGPNYTPSAWRRGHRVDFGKIFDGTAPFNKINWGSSPTPGDDGISFSNETIGSEPFATPPSSPSQTPVINVNVNVGGTNVNIGDTNVSKGSGTPTSSQSVDMSTDTSDLDTSDIDTNNQTNGDINTNDNSNNVDGSLSDVDSRVEDDDGVSDTESTNGNDSGKTTSTEENGDPSGPDILAAVRKHLDTVYPGENGGSTEGPLPANQNLGNVIHDVEQNGSAKETIITPGDTGPTDSSSSVDADADVEDTSDTDSGIGDDDGVSDTESTNGNNSGKTTSTEENGDPSGPDILAAVRKHLDTVYPGENGGSTEGPLPANQNLGNVIHDVEQNGAAQETIITPGDTESTDTSSSVNANADLEDVSDADSGFGDDDGISDTESTNGNDSGKNTPVGDGGTPSGPDILAAVRKHLDTVYPGENGGSTERPLPANQNLGDIIHDVEQNGSAKETVVSPYRGGGGNTSSPIGLASLLPATPSTPLMTTPRTNGKAAASSLMIKGGETQAKLVKNGGNIPGETTLAELLPRLRGHLDKVFTSDGKFTNLNGPQLGAIIDQFRKETGSGGIIAHTDSVPGENGTASPLTGSSGEKVSLYDAAKNVTQALTSVTNKVTLAMQGQKLEGIINNNNTPSSIGQNLFAAARATTQSLSSLIGTVQ

SEQ ID NO: 59 - DNA, 780 bp, Chlamydia muridarum
gtgagtatgtatataaaaagaaagaaagcttggatgactttcttagcaattgtctgtagtttctgtttggcgggctgttcaaaagagagcaaagactctgttagtgaaaaatttattgtaggaactaacgcaacgtatcctccttttgagtttgttgatgaaagaggtgagacggttggctttgatattgatttagctagggagattagtaaaaagctagggaaaaaattagaagtccgagaatttgcttttgatgcactcgttctcaatttaaaacagcatcgtattgatgcaattatggcaggggtgtccattacgtcttctcgattgaaagaaattttgatgattccctactatggcgaagaaataaagagtttggttttagtgtttaaggatggagactcaaagtctttaccactagatcagtataattctgttgctgttcaaactggcacgtaccaagaggaatatttacagtctcttccaggggtgcgtattcgctcttttgatagtactttagaagtgcttatggaagttttgcatagcaagtctcctatagctgttttagaaccgtctattgcgcaggtcgttttaaaagattttccgacgctcactactgaaacgatagatcttcctgaagataaatgggttttagggtatggaattggagttgcttctgatcgaccatctctagcttctgatatagaagctgctgtacaagagatcaagaaagaaggagtgttagcagagttagagcaaaaatggggtttgaacggctaa

SEQ ID NO: 60 - protein, 259 aa, Chlamydia muridarum
MSMYIKRKKAWMTFLAIVCSFCLAGCSKESKDSVSEKFIVGTNATYPPFEFVDERGETVGFDIDLAREISKKLGKKLEVREFAFDALVLNLKQHRIDAIMAGVSITSSRLKEILMIPYYGEEIKSLVLVFKDGDSKSLPLDQYNSVAVQTGTYQEEYLQSLPGVRIRSFDSTLEVLMEVLHSKSPIAVLEPSIAQVVLKDFPTLTTETIDLPEDKWVLGYGIGVASDRPSLASDIEAAVQEIKKEGVLAELEQKWGLNG

SEQ ID NO: 61 - DNA, 384 bp, Chlamydia trachomatis
atggaagaaaaaggcatcttacaattggttgaaatttcgcgagcaatggctttacagggagtttgtccttggactaatttacagagtgtggagtctatgttgcagtatatagcaggggagtgtcaggagttggctgatgctgtacaagaaaataaagcttcgttggaaatcgcttcggaagccggagacgtacttactttagtattgaccttgtgtttcttgctagaaagagaaggaaagcttaaagctgaagaagtatttgtagaagctttggctaagttgcgtcgtcgatctcctcatgtttttgatcctcataatcaaatttctttagaacaggctgaagaatactgggctcgtatgaaacagcaagaaaaaatttcttaa

SEQ ID NO: 62 - protein, 127 aa, Chlamydia trachomatis
MEEKGILQLVEISRAMALQGVCPWTNLQSVESMLQYIAGECQELADAVQENKASLEIASEAGDVLTLVLTLCFLLEREGKLKAEEVFVEALAKLRRRSPHVFDPHNQISLEQAEEYWARMKQQEKIS

SEQ ID NO: 63 - DNA, 1179 bp, Chlamydia trachomatis
atggattactacacgatattgggtgtagcgaagactgctactcctgaagaaataaagaaagcttaccgtaagctcgctgtaaagtaccatccagataagaatcctggggatgctgaagcggagcgacgctttaaagaagtttctgaagcctatgaagtattaggtgatgcgcagaagcgggagtcatatgatcgttacggcaaagacggtccatttgctggtgctggaggattcggtggcgctggcatggggaatatggaagacgctttgcgaacatttatgggagcttttggcggcgatttcggtggtaatggaggcggtttctttgaagggctttttggaggacttggagaagctttcggaatgcgtggaggctcagaaagttctcgacaaggagctagtaagaaggtgcatattacgctgtccttcgaggaggcggcaaaaggtgttgaaaaagaacttcttgtttcaggctataaatcttgtgatgcttgttctggtagtggagccaatactgctaaaggtgtaaaagtttgtgatcgatgcaagggctctggtcaggtagtgcaaagccgaggctttttctccatggcttctacttgccctgattgtagtggtgaaggtcgggttatcacagatccttgttcagtttgtcgtgggcagggacgtatcaaggataaacgtagcgtccatgttaatatcccagctggagtcgattctgggatgagattaaagatggaaggctatggagatgctggccaaaatggagcgcctgcaggggatctgtatgtttttattgatgtagagcctcatcctgttttcgagcgccatggggatgatttagttttagagcttcctattggatttgttgatgcggctttagggatcaagaaggaaatccctacactcttaaaagaaggtacttgccgtttgagtatcccagaagggattcagagcggaacagttcttaaagttagagggcagggattccctaatgtgcatgggaaatccagaggagatcttttagtaagagtatctgtggagactccccagcacctatctaatgaacaaaaagatttattgagacagtttgctgctacggagaaggctgaaaatttccctaagaaacggagtttcttagacaaaatcaaaggttttttttctgactttgctgtatag

SEQ ID NO: 64 - protein, 392 aa, Chlamydia trachomatis
MDYYTILGVAKTATPEEIKKAYRKLAVKYHPDKNPGDAEAERRFKEVSEAYEVLGDAQKRESYDRYGKDGPFAGAGGFGGAGMGNMEDALRTFMGAFGGDFGGNGGGFFEGLFGGLGEAFGMRGGSESSRQGASKKVHITLSFEEAAKGVEKELLVSGYKSCDACSGSGANTAKGVKVCDRCKGSGQVVQSRGFFSMASTCPDCSGEGRVITDPCSVCRGQGRIKDKRSVHVNIPAGVDSGMRLKMEGYGDAGQNGAPAGDLYVFIDVEPHPVFERHGDDLVLELPIGFVDAALGIKKEIPTLLKEGTCRLSIPEGIQSGTVLKVRGQGFPNVHGKSRGDLLVRVSVETPQHLSNEQKDLLRQFAATEKAENFPKKRSFLDKIKGFFSDFAV

SEQ ID NO: 65 - DNA, 366 bp, Chlamydia trachomatis
atgaataaaaaactccaagatctgtctaaactgctcactattgagcttttcaagaaacgtacacggttggaaacagtaaaaaaagcgctctccacaatagaacatcgcttacaacaaatacaggagcacatcgcgaaaatttccttaacaaggcacaaacaattcctatgtcggtcatatacccatgaatatgaccaacatttagaacatttacaaagagagcaaacttctctatataaacagcatcagaccctgaaaacgtctttgaaagatgcttatggcgacatacaaaaacaactagaccaaagaaaaattatcgaaaagatccatgacagtaaatatcctataaagagcgcgaataactaa

SEQ ID NO: 66 - protein, 121 aa, Chlamydia trachomatis
MNKKLQDLSKLLTIELFKKRTRLETVKKALSTIEHRLQQIQEHIAKISLTRHKQFLCRSYTHEYDQHLEHLQREQTSLYKQHQTLKTSLKDAYGDIQKQLDQRKIIEKIHDSKYPIKSANN

SEQ ID NO: 67 - DNA, 1275 bp, Chlamydia trachomatis
atgaaacatgctctcattgttggctcaggtattgccggcctttctgccgcgtggtggctacacaaacgattccctcatgtgcagctgtctattctagaaaaagagtctcgatctggagggctaattgtcacagagaaacaacaagggttttccctcaatatgggccctaaaggttttgttttagctcatgatgggcaacacacccttcacctcattcagtctttaggcctagcagacgagctattatatagctctccagaggctaaaaaccgctttatccactataataataaaacccgaaaagtctcgccttggactattttcaaacaaaatctccctctctcttttgctaaggatttctttgcgcgtccttacaaacaagacagctccgtggaagccttctttaaaagacacagttcttccaagcttagaagaaatcttttaaatcccattagcattgctattcgtgcaggacatagtcatatattgtctgcacagatggcttacccagaattaacacgaagagaagctcaaacaggatcgttgttacgtagttatctcaaagattttcctaaagagaaacgcacaggcccttatttagctaccttgcggtctgggatgggaatgctaacccaggctttgcatgataaattgcctgctacctggtatttttctgcacccgtcagcaaaatccgtcagttggcgaatgggaaaatttctctttcatctcctcaaggagaaataacgggagatatgctcatttatgctgggtccgtgcacgatctcccttcctgtctagaagggatccctgaaaccaagcttatcaagcaaacgacttcatcttgggatctctcttgtgtatctttaggatggcatgcatccttccctatccctcatggatatggcatgcttttcgctgatacgcctcccttattagggatcgtgtttaatacggaagtgttccctcaacccgagcggcctaatacaatagtctctcttcttttagaaggtcgatggcaccaagaagaagcgtatgctttctcactagcagctatttctgagtacctgcaaatttacactcctccccaagctttctcactattctctcctcgagagggacttccccaacaccatgttggatttatccaatcccgccaacgccttctatctaaacttcctcacaatataaaaattgtagggcagaattttgcaggtccaggtctcaaccgcgctacagcgtctgcttataaagctatagcttctttactatcatga

SEQ ID NO: 68 - protein, 424 aa, Chlamydia trachomatis
MKHALIVGSGIAGLSAAWWLHKRFPHVQLSILEKESRSGGLIVTEKQQGFSLNMGPKGFVLAHDGQHTLHLIQSLGLADELLYSSPEAKNRFIHYNNKTRKVSPWTIFKQNLPLSFAKDFFARPYKQDSSVEAFFKRHSSSKLRRNLLNPISIAIRAGHSHILSAQMAYPELTRREAQTGSLLRSYLKDFPKEKRTGPYLATLRSGMGMLTQALHDKLPATWYFSAPVSKIRQLANGKISLSSPQGEITGDMLIYAGSVHDLPSCLEGIPETKLIKQTTSSWDLSCVSLGWHASFPIPHGYGMLFADTPPLLGIVFNTEVFPQPERPNTIVSLLLEGRWHQEEAYAFSLAAISEYLQIYTPPQAFSLFSPREGLPQHHVGFIQSRQRLLSKLPHNIKIVGQNFAGPGLNRATASAYKAIASLLS

SEQ ID NO: 69 - protein, 6 aa, artificial sequence
  linker
GSGGGG

SEQ ID NO: 70 - protein, 11 aa, artificial sequence
  polycationic polymer peptide
KLKLLLLLKLK